SEQUENCE LISTING (61 sequences)

SEQ ID NO: 1 - DNA, 35 bp, Artificial Sequence
  Description of Artificial Sequence Synthetic primer
cgaagaaattcgatggcttccaaaagagctctggt

SEQ ID NO: 2 - DNA, 35 bp, Artificial Sequence
  Description of Artificial Sequence Synthetic primer
cagactcgagctgcttcacatactactgctgaggt

SEQ ID NO: 3 - DNA, 20 bp, Artificial Sequence
  Description of Artificial Sequence Synthetic primer
ttttggatgcaaggtcacaa

SEQ ID NO: 4 - DNA, 20 bp, Artificial Sequence
  Description of Artificial Sequence Synthetic primer
ctccacaatggctagtgcaa

SEQ ID NO: 5 - protein, 19 aa, Artificial Sequence
  Description of Artificial Sequence Synthetic peptide
QNLSESPMVKEILKEQESR

SEQ ID NO: 6 - DNA, 55 bp, Artificial Sequence
  Description of Artificial Sequence Synthetic oligonucleotide
tgtcactgttgcaggcttggttcaagagaccaagcctgcaacagtgacttttttc

SEQ ID NO: 7 - DNA, 59 bp, Artificial Sequence
  Description of Artificial Sequence Synthetic oligonucleotide
acagtgacaacgtccgaaccaagttctctggttcggacgttgtcactgaaaaaagagct

SEQ ID NO: 8 - DNA, 24 bp, Artificial Sequence
  Description of Artificial Sequence Synthetic primer
aatactgtgtactctcaagatgga

SEQ ID NO: 9 - DNA, 22 bp, Artificial Sequence
  Description of Artificial Sequence Synthetic primer
ttacgcctagcgtgtagactct

SEQ ID NO: 10 - DNA, 23 bp, Artificial Sequence
  Description of Artificial Sequence Synthetic primer
caattagatctaatgggacggag

SEQ ID NO: 11 - DNA, 24 bp, Artificial Sequence
  Description of Artificial Sequence Synthetic primer
tgctctgagaggggtcttctgaag

SEQ ID NO: 12 - DNA, 23 bp, Artificial Sequence
  Description of Artificial Sequence Synthetic primer
tctataaccttgcaaagacacct

SEQ ID NO: 13 - DNA, 24 bp, Artificial Sequence
  Description of Artificial Sequence Synthetic primer
agaagcgatcttggtccttacctg

SEQ ID NO: 14 - DNA, 38 bp, Artificial Sequence
  Description of Artificial Sequence Synthetic oligonucleotide
gtctactggataagagcccgaggccgaggctgagaccg

SEQ ID NO: 15 - DNA, 39 bp, Artificial Sequence
  Description of Artificial Sequence Synthetic oligonucleotide
ctcagcctcggcctcgggctcttatccagtagacagggt

SEQ ID NO: 16 - DNA, 38 bp, Artificial Sequence
  Description of Artificial Sequence Synthetic oligonucleotide
gtctactggatgggagcccgattccgaggctgagaccg

SEQ ID NO: 17 - DNA, 39 bp, Artificial Sequence
  Description of Artificial Sequence Synthetic oligonucleotide
ctcagcctcggaatcgggctcccatccagtagacagggt

SEQ ID NO: 18 - DNA, 25 bp, Mus musculus
taagagctcaaggctgagacctgcc

SEQ ID NO: 19 - DNA, 25 bp, Homo sapiens
taagagcccgaggccgaggctgagg

SEQ ID NO: 20 - DNA, 22 bp, Artificial Sequence
  Description of Artificial Sequence Synthetic primer
catcagcggaaatgaccttctg

SEQ ID NO: 21 - DNA, 22 bp, Artificial Sequence
  Description of Artificial Sequence Synthetic primer
cttgaaagagtccacaggaacc

SEQ ID NO: 22 - DNA, 24 bp, Artificial Sequence
  Description of Artificial Sequence Synthetic primer
gcaatacagcagctaccaatgctg

SEQ ID NO: 23 - DNA, 24 bp, Artificial Sequence
  Description of Artificial Sequence Synthetic primer
ggctaagatctacagctgccttgt

SEQ ID NO: 24 - DNA, 25 bp, Artificial Sequence
  Description of Artificial Sequence Synthetic primer
catggactacaaggacgacgatgac

SEQ ID NO: 25 - DNA, 24 bp, Artificial Sequence
  Description of Artificial Sequence Synthetic primer
aatcggagctgtattctcccgaag

SEQ ID NO: 26 - DNA, 24 bp, Artificial Sequence
  Description of Artificial Sequence Synthetic primer
ttgaaaaactctccacggtgtact

SEQ ID NO: 27 - DNA, 24 bp, Artificial Sequence
  Description of Artificial Sequence Synthetic primer
tacgggtcaaacttcacagagaat

SEQ ID NO: 28 - DNA, 21 bp, Artificial Sequence
  Description of Artificial Sequence Synthetic primer
gtatgctctgatgccatccat

SEQ ID NO: 29 - DNA, 21 bp, Artificial Sequence
  Description of Artificial Sequence Synthetic primer
tgatgcacatagcagcaactc

SEQ ID NO: 30 - DNA, 19 bp, Artificial Sequence
  Description of Artificial Sequence Synthetic primer
ctccctccatgagggtctg

SEQ ID NO: 31 - DNA, 20 bp, Artificial Sequence
  Description of Artificial Sequence Synthetic primer
tcttcggcttcgagggtaaa

SEQ ID NO: 32 - DNA, 20 bp, Artificial Sequence
  Description of Artificial Sequence Synthetic primer
tttcgcaacgggtttgccgc

SEQ ID NO: 33 - DNA, 23 bp, Artificial Sequence
  Description of Artificial Sequence Synthetic primer
aaggtcgcctctagctcctgtag

SEQ ID NO: 34 - DNA, 24 bp, Artificial Sequence
  Description of Artificial Sequence Synthetic primer
aatatctacggactcatcgctccc

SEQ ID NO: 35 - DNA, 24 bp, Artificial Sequence
  Description of Artificial Sequence Synthetic primer
gcatacacactcccatagacagac

SEQ ID NO: 36 - DNA, 22 bp, Artificial Sequence
  Description of Artificial Sequence Synthetic primer
gccggggctgcgcttggctttg

SEQ ID NO: 37 - DNA, 24 bp, Artificial Sequence
  Description of Artificial Sequence Synthetic primer
tggcggtgcttccgggacatgagc

SEQ ID NO: 38 - DNA, 24 bp, Artificial Sequence
  Description of Artificial Sequence Synthetic primer
gaaactctccctgactctactccc

SEQ ID NO: 39 - DNA, 20 bp, Artificial Sequence
  Description of Artificial Sequence Synthetic primer
cttgctgctcttcctctggt

SEQ ID NO: 40 - DNA, 21 bp, Artificial Sequence
  Description of Artificial Sequence Synthetic primer
aactccaccctcatcaaccca

SEQ ID NO: 41 - DNA, 21 bp, Artificial Sequence
  Description of Artificial Sequence Synthetic primer
aagcccacacctttcaggaca

SEQ ID NO: 42 - DNA, 24 bp, Artificial Sequence
  Description of Artificial Sequence Synthetic primer
tccaaagggaacaactgcctcgat

SEQ ID NO: 43 - DNA, 22 bp, Artificial Sequence
  Description of Artificial Sequence Synthetic primer
agctgtgcttggctggaacttt

SEQ ID NO: 44 - DNA, 22 bp, Artificial Sequence
  Description of Artificial Sequence Synthetic primer
acgcttggtggttgattctgga

SEQ ID NO: 45 - DNA, 24 bp, Artificial Sequence
  Description of Artificial Sequence Synthetic primer
aacttgcttcctgtttctgagggc

SEQ ID NO: 46 - DNA, 22 bp, Artificial Sequence
  Description of Artificial Sequence Synthetic primer
acgacatcactggctgacactt

SEQ ID NO: 47 - DNA, 22 bp, Artificial Sequence
  Description of Artificial Sequence Synthetic primer
tccaaaggcacttgactgctga

SEQ ID NO: 48 - DNA, 24 bp, Artificial Sequence
  Description of Artificial Sequence Synthetic primer
tgagagggaaatcgtgcgtgacat

SEQ ID NO: 49 - DNA, 24 bp, Artificial Sequence
  Description of Artificial Sequence Synthetic primer
accgctcgttgccaatagtgatga

SEQ ID NO: 50 - DNA, 24 bp, Artificial Sequence
  Description of Artificial Sequence Synthetic primer
aagctgaccctgaagttcatctgc

SEQ ID NO: 51 - DNA, 24 bp, Artificial Sequence
  Description of Artificial Sequence Synthetic primer
caccttgatgccgttcttctgctt

SEQ ID NO: 52 - DNA, 20 bp, Artificial Sequence
  Description of Artificial Sequence Synthetic primer
tttcgcaacgggtttgccgc

SEQ ID NO: 53 - DNA, 23 bp, Artificial Sequence
  Description of Artificial Sequence Synthetic primer
aaggtcgcctctagctcctgtag

SEQ ID NO: 54 - DNA, 24 bp, Artificial Sequence
  Description of Artificial Sequence Synthetic primer
tggcacagtccctatttcttcgct

SEQ ID NO: 55 - DNA, 24 bp, Artificial Sequence
  Description of Artificial Sequence Synthetic primer
aatgcagccaatagcaccacacag

SEQ ID NO: 56 - DNA, 24 bp, Artificial Sequence
  Description of Artificial Sequence Synthetic primer
ggcgtatggagcagggttactttc

SEQ ID NO: 57 - DNA, 22 bp, Artificial Sequence
  Description of Artificial Sequence Synthetic primer
aatgctctgcgtgtagcggttg

SEQ ID NO: 58 - DNA, 24 bp, Artificial Sequence
  Description of Artificial Sequence Synthetic primer
agcgagccttgttgacatgagaga

SEQ ID NO: 59 - DNA, 24 bp, Artificial Sequence
  Description of Artificial Sequence Synthetic primer
tcaagattgcttggcttggttggg

SEQ ID NO: 60 - DNA, 24 bp, Artificial Sequence
  Description of Artificial Sequence Synthetic primer
tcaacgttaagctagccctggaca

SEQ ID NO: 61 - DNA, 24 bp, Artificial Sequence
  Description of Artificial Sequence Synthetic primer
tctgtacaggaatggtgatgcggt

(19) United States
(12) Patent Application Publication
(10) Pub. No.: US 2006/0275744 A1
(43) Pub. Date: Dec. 7, 2006

(54) Modified human embryonic stem cells and methods of use to treat neuron-associated disorders

(75) Inventors: Asa Abeliovich, New York, NY (US); Cecile Martinat, Paris (FR)

Correspondence Address: WILMER CUTLER PICKERING HALE AND DORR LLP;COLUMBIA UNIVERSITY, 399 PARK AVENUE, NEW YORK, NY, 10020, US

(73) Assignee: THE TRUSTEES OF COLUMBIA UNIVERSITY IN THE CITY OF NEW YORK, New York, NY (US)

(21) Appl. No.: 11/258,359

(22) Filed: Oct. 24, 2005

Related U.S. Application Data
(63) Continuation-in-part of application No. 11/196,376, filed on Aug. 2, 2005.
(60) Provisional application No. 60/598,815, filed on Aug. 2, 2004.
(60) Provisional application No. 60/621,381, filed on Oct. 22, 2004.

(51) Int. Cl. C12N 5/06; C12N 5/08; C12Q 1/00; C12N 15/09

(52) U.S. Cl. 435/4; 435/455; 435/354; 435/368

(57) ABSTRACT

The invention is directed to methods for promoting the differentiation and maturation of embryonic and adult stem cells to dopamine neurons by increasing expression of the transcription factors Nurr1 and PitX3 in the stem cells. The invention provides embryonic and adult stem cells expressing Nurr1 and PitX3 from nucleic acid vectors, and dopamine neurons differentiated therefrom, and methods for treating a neuron-associated disorder, such as Parkinson's Disease, using the cells of the invention. The invention encompasses screening assays using the cells of the invention to identify compounds capable of inducing differentiation of stem cells to dopamine neurons.

This application is a continuation-in-part of U.S. patent application Ser. No. 11/196,376, filed Aug. 2, 2005, which claims priority to U.S. Provisional Application No. 60/598,815, filed Aug. 2, 2004; this application also claims priority to U.S. Provisional Application No. 60/621,381, filed Oct. 22, 2004, all of the foregoing applications are hereby incorporated by reference in their entireties.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described herein.

### FIELD OF THE INVENTION

The present invention relates to modified stem cells and uses thereof to treat neuron-associated disorders.

### BACKGROUND OF THE INVENTION

Parkinson's disease (PD) is a progressive neurodegenerative disorder characterized by rigidity, slowed movement, gait difficulty, and tremors (Dauer and Przedborski 2003). The pathological hallmark of PD is the relatively selective loss of dopamine neurons (DN) in the substantia nigra pars compacta in the ventral midbrain. Although the cause of neurodegeneration in PD is unknown, a Mendelian inheritance pattern is observed in approximately 5% of patients, suggesting a genetic factor. Pathological analyses of PD substantia nigra have correlated cellular oxidative stress and altered proteasomal function with PD. Extremely rare cases of PD have been associated with the toxin 1-methyl-4-phenyl-1,2,3,6-tetrahydropyridine (MPTP), which is taken up specifically by dopamine neurons through the dopamine transporter and is thought to induce cellular oxidative stress. Population-based epidemiological studies have further supported roles for genetic and environmental mechanisms in the etiology of PD (Dauer and Przedborski 2003; Jenner 2003).

The identification of several genes that underlie familial forms of PD has allowed molecular dissection of mechanisms of dopamine neuron survival. Autosomal dominant mutations in α-synuclein (GENEBANK Accession Number NM_{—}000345) lead to a rare familial form of PD (Polymeropoulos et al. 1997), and there is evidence that these mutations generate abnormal protein aggregates (Goldberg and Lansbury 2000) and proteasomal dysfunction (Rideout et al. 2001). A majority of patients with sporadic PD harbor prominent intracytoplasmic inclusions, termed Lewy bodies, enriched for α-synuclein (Spillantini et al. 1998), as well as neurofilament protein (Trojanowski and Lee 1998). Mutations in a second gene, Parkin (GENEBANK Accession Number AB009973), lead to autosomal recessive PD (Hattori et al. 2000). Parkin is a ubiquitin ligase that appears to participate in the proteasome-mediated degradation of several substrates (Staropoli et al. 2003).

Homozygous mutations in a third gene, DJ-1 (GENEBANK Accession Number AB073864), were recently associated with autosomal recessive primary parkinsonism (Bonifati et al. 2003). Furthermore, homozygous mutations in the DJ-1 gene have recently been described in two families with autosomal recessive PD, one of which is a large deletion that likely leads to loss of its function. DJ-1 encodes a ThiJ domain protein of 189 amino acids that is broadly expressed in mammalian tissues (Nagakubo et al. 1997). Interestingly, DJ-1 was independently identified in a screen for human endothelial cell proteins that are modified with respect to pI in response to sublethal doses of paraquat (Mitsumoto and Nakagawa 2001; Mitsumoto et al. 2001), a toxin which generates reactive oxygen species (ROS) within cells and has been associated with dopamine neuron toxicity (McCormack et al. 2002). Gene expression of a yeast homologue of DJ-1, YDR533C, is upregulated in response to sorbic acid (de Nobel et al. 2001), an inducer of cellular oxidative stress. These data suggest a causal role for DJ-1 in the cellular oxidative stress response.

Surprisingly, animal models that harbor genetic lesions that mimic inherited forms of human PD, such as homozygous deletions in Parkin (Goldberg et al. 2003; Itier et al. 2003) or overexpression of α-Synuclein (Masliah et al. 2000; Giasson et al. 2002; Lee et al. 2002), have failed to recapitulate the loss of dopamine cells. An alternative approach, the genetic modification of midbrain dopamine neurons in vitro (Staropoli et al. 2003), is potentially useful but limited by the difficulty and variability in culturing primary post-mitotic midbrain neurons. Other studies have focused on immortalized tumor cell lines, such as neuroblastoma cells, but these may not accurately model the survival of postmitotic midbrain neurons.

Models of neurodegenerative diseases are essential for the development and validation of effective therapies to treat these diseases. Cellular models are particularly attractive, as they are more readily manipulated with genetic and pharmacological interventions, and can be miniaturized for high-throughput screening of drugs. Whole-animal models are less desirable, as they are not easily adapted for the screening of therapeutics, they display much variance, and they are less reproducible. While cellular-model approaches to studying neurodegenerative disorders are desirable, they are often limited by the lack of available primary neurons. Neurons are post-mitotic (non-dividing) cells, and, therefore, are difficult to obtain in large numbers.

Midbrain dopamine neurons (DNs) play an essential role in the regulation of voluntary movement, and their degeneration is associated with Parkinson's disease (PD) and related neurodegenerative disorders. Although symptomatic therapies exist for Parkinson's disease (PD) that improve the motor function of patients, no treatments are available that slow the relentless course of the disease. Given the relatively specific loss of dopamine neurons (DNs) in PD, cell replacement therapies offer a promising treatment strategy (Dauer and Przedborski, 2003). However, major hurdles remain: the current state-of-the-art in dopamine cell therapy is of limited efficacy. In two placebo-controlled, prospective trials with fetal-derived midbrain cells transplanted into the striatum, patients experienced no subjective benefit, although some younger patients did appear to improve by certain objective measures (Freed et al., 2000; Olanow et al., 2003). A significant percentage of treated patients suffered from dyskinesias in both studies. These results suggest that transplanted cells require further cues to function in the context of an intact CNS, and emphasize the importance of identifying critical developmental signals for dopamine neurons.

Although some factors in the early development of dopamine neurons have been identified, the mechanisms determining the development of fully functional DNs remain poorly understood. Dopamine neuron generation in the mouse midbrain may be broadly divided into several stages (FIG. 1) (Wallen and Perlmann, 2003). Initially, (mouse post-implantation embryo days 8-10; E8-10) multipotent, mitotically active periventricular neuronal precursors are specified to become midbrain neuroblasts, characterized by the expression of a subset of homeobox genes (such as Lmx1b, Aldh1, and Engrailed 1 and 2). Next (E10.5), in response to the activity of environmental signals such as sonic hedgehog (SHH) and fibroblast growth factor-8 (FGF-8) and intrinsic signaling molecules such as Nurr1, these neuroblasts become post-mitotic and are specified to express “early” dopamine markers such as tyrosine hydroxylase (TH), the rate-limiting enzyme in dopamine synthesis, and the transcription factor PitX3. Late differentiation (E12-15) is characterized by the expression of several synaptic markers and the dopamine transporter (DAT). Furthermore, synaptogenesis and vesicular depolarization-induced dopamine release is observed. Finally, several studies have suggested that subsequent interactions with target tissues, such as the striatum, play a role (Perrone-Capano et al., 2000).

The functional role of the orphan nuclear receptor transcription factor Nurr1 in midbrain dopamine neurons was first demonstrated in Nurr-1 deficient ‘knockout’ mice, in which these cells are absent at birth (Le et al., 1999; Zetterstrom et al., 1997). The earliest defect observed in Nurr-1 deficient midbrain dopamine neurons is the absence of phenotypic markers including TH (at E11), although other early markers of dopamine neurons (such as PitX3 and Lmx1b) remain unaltered. Nurr1 deficient DNs may also be defective in migration and target innervation (although this point has been challenged (Witta et al., 2000)), and by birth these cells are lost. Overexpression of Nurr1 in hippocampal progenitors has been found to lead to increased TH expression (Sakurada et al., 1999), but other genes were not apparently induced and cells appeared not to differentiate. Similarly, Nurr1 overexpression in rat embryonic midbrain precursors appeared to increase TH expression, but these cells failed to function in vivo in the rescue of 6-hydroxydopamine (6-OHDA, a dopamine neuron specific toxin) (Kim et al., 2003). Taken together, these data suggest that Nurr1 plays an essential role at an early stage of dopamine neuron development but is not sufficient.

The role of Nurr1 in ‘late’ midbrain DN differentiation, survival, and function remain unclear. There is a report of increased sensitivity to the dopamine neuron specific toxin MPTP in mice that are heterozygous for the deletion of Nurr1, and a report suggesting a genetic association between human alleles of Nurr1 and PD (Le et al., 2003).

The regulation of Nurr1 activity in vivo remains unclear. Although Nurr1 is an orphan nuclear receptor and has therefore been hypothesized to be activated by an unknown ligand, it appears from crystal structure data that Nurr1 lacks a cavity for ligand binding, and therefore no true ligand may exist (Wang et al., 2003). Like a number of nuclear receptors, Nurr1 dimerizes with the retinoic X receptor (RXR) (Wallen-Mackenzie et al., 2003; Zennou et al., 2001). Interestingly, such heterodimers are dependent on RXR-specific ligands for activity, such as docosahexanoic acid (DHA), an endogenous ligand present in the mammalian CNS. RXR-specific ligands do appear to increase the generation or survival of midbrain embryonic cultures, although it is not clear whether this is through a Nurr1-dependent mechanism or another RXR-related pathway (Wallen-Mackenzie et al., 2003). Nurr1 is also capable of binding DNA as a monomer or as a homodimer and as such appears to function constitutively, although homodimer function may be further activated by a PKA pathway (Maira et al., 1999). The function of these forms of Nurr1 in vivo remains undetermined.

Mouse knockout of a gene that encodes a second early dopamine neuron marker, Lmx1b, also leads to the eventual loss of TH positive cells in the midbrain (at E16.5) (Smidt et al., 2000). At an earlier time point (E12.5), however, Nurr1 and TH expression appear unaltered in the midbrain, although PitX3 expression is absent. Thus, Lmx1b appears to be required for ‘late’ events in the differentiation and survival of these cells. A third strain of mutant mice, the naturally occurring aphakia mice that are mutated in PitX3, also display initially normal midbrain expression of TH (at E12), but by birth there is a remarkably specific loss of substantia nigra TH expression, whereas TH is reduced to a lesser degree in the adjacent ventral segmental area (Nunes et al., 2003; van den Munckhof et al., 2003). These data have led to the suggestion that two independent intrinsic pathways are required for the specification of SN dopamine neurons: A Nurr1 pathway that is required for the expression of TH, and a second pathway that involves both Lmx1b and PitX3 and is necessary for the terminal differentiation and/or survival of SN DNs (FIG. 2).

The effect of the local cellular environment on the differentiation of DN precursors may be exerted through diffusible factors and/or through direct cell-cell contacts (FIG. 2). Early developmental specification of the midbrain neuroepithelium is thought to be guided by positional cues from the floor plate in the form of Sonic Hedgehog, and the midbrain-hindbrain (MHB) junction in the form of FGF-8 (Ye et al., 1998). These signals may establish a Cartesian coordinate system for positional information instructive in the generation of subsequent proliferating DA precursors and post-mitotic cells. In addition, the TGFβ/Nodal signaling pathway may play a role in the early specification of DNs. These early extrinsic cues, in turn, such as FGF-8, are established by the coordinated activity of a network of intrinsic transcription factors including Pax2 (Ye et al., 2001).

Several candidate factors have been implicated in late events in the specification and maturation of functional dopamine neurons. For instance, glial cell-line-derived neurotrophic factor (GDNF) and brain-derived neurotrophic factor (BDNF) can enhance the survival of DNs, and furthermore these factors may influence the late differentiation of DNs, and specifically synaptic maturation of primary DNs (Feng et al., 1999). Similarly, Wnts are secreted factors that modulate early events in neuron development as well as synapse formation and maturation elsewhere in the CNS (Goda and Davis, 2003). In developing midbrain dopamine precursors, there is evidence that the ‘canonical’ Wnt pathway functions upstream of Nurr1 signaling to potentiate the proliferation of mitotic precursors (Castelo-Branco et al., 2003). In contrast, Wnt 5a, which is thought to signal through a non-canonical pathway and may inhibit the canonical Wnt pathway, appears to potentiate the generation of DN at a later step, perhaps through the induction of PitX3 expression (Castelo-Branco et al., 2003).

It is instructive to compare DN specification to the pathways for specification of other monoaminergic neuronal fates in the mammalian midbrain. Serotonergic neurons (SN) arise from ventral precursors in the hindbrain, caudal to the DN progenitors. Similar to DNs, the SNs require Sonic Hedgehog signal from the floor plate (Ye et al., 1998). FGF4 appears to similarly be necessary for the generation of SNs and is thought to specify the caudal location of these cells in a similar Cartesian manner as does FGF-8 in the midbrain DNs. Several transcription factors act in a coordinated fashion to specify SN fate. Nkx2.2, a homeobox domain protein, is required ‘early’ and may function primarily to suppress the Paired-type transcription factor Phox2b and prevent specification of a motor neuron fate (Pattyn et al., 2003). Pet-1, an ETS class transcription factor specific for serotonergic cells, along with Lmx1b, a LIM homeodomain protein (also required for ‘late’ events in dopamine neuron specification, function coordinately with Nkx2.2 to specify SNs (Cheng et al., 2003).

Noradrenergic cells in the locus ceruleus (LC) arise in the dorsal hindbrain and project broadly in the CNS (Goridis and Rohrer, 2002). Like other dorsal cell fates, the early dorsalization process requires BMP signaling, but interestingly there is also evidence for BMP signaling at later time points in development, including post-mitotic events and synapse formation. Several transcription factors regulate the differentiation of LC norepinephrine cells, including Phox2b, Phox2a and Mash1. Of these, only Phox2b appears to be both necessary and sufficient for the specification of hindbrain precursors to express TH. It is interesting that two Paired-like homeobox transcription factors, Phox2b and PitX3, appear to be both necessary and sufficient to encode two related but different fates in the MHB junction, suggesting a transcription factor network regulating cell fate determination akin to the network in the spinal cord (Dasen et al., 2003).

A critical issue with regard to cell replacement therapy is the availability of appropriate donor cells. Fetal-derived dopamine neurons have been used in most of the previously attempted cell replacement clinical studies, but such cells are of limited availability and are subject to ethical debate. In contrast, stem cell-derived dopamine neurons, either from embryonic stem (ES) cells or from neuronal stem cells (NSC) offer the potential for a limitless supply, as stem cells by definition are self-renewing (Freed, 2002).

Embryonic stem cells (ES cells), derived from early embryos, are “immature” cells that have the potential to develop into different cell types including DNs (Bjorklund et al., 2002; Kim et al., 2002). The in vitro differentiation of ES cells provides new perspectives for studying the cellular and the molecular mechanisms of neuronal development. Murine ES-derived dopamine neurons have been shown to follow much the same early differentiation pattern as endogenous dopamine neurons with respect to a number of early markers. Furthermore, transplantation of murine ES-derived dopamine neurons appear to function in an animal model of PD, 6-hydroxydopamine treated rats.

Several studies have investigated the role of ‘early’ extrinsic factors, including Sonic Hedgehog and FGF-8, in ES differentiation protocols, and these suggest that these factors do potentiate the generation of DNs (Kim et al., 2002; Lee et al., 2000). Furthermore, overexpression of Nurr1, an ‘early’ intrinsic factor, appears to potentiate the generation of early markers of DNs (Chung et al., 2002; Kim et al., 2002), particularly TH. One caveat to the interpretation of the study from Kim et al., however, is that they do not compare the Nurr1-transfected ES clone to control vector-transfected cells, limiting the interpretation of these data. One study (Rolletschek et al., 2001) did investigate the efficacy of a cocktail of growth factors (including BDNF and GDNF) on the maturation of ES-derived dopamine neurons, but this study failed to observe an effect on dopamine levels of this cocktail, and did not include a kinetic analysis of the roles of these factors.

At present, a major limitation in PD is the lack of a reliable animal or cellular model system for this disease. Mouse genetic models of disease are often limited by the inherent variability of animal experiments, the limited mouse lifespan, and by difficulties in manipulating whole animals. For instance, genetic rescue experiments and toxicological dose-response studies are impractical in whole animals. Furthermore, genetic cell models are more readily amenable to molecular dissection of disease mechanism. Thus, genetically altered, ES-derived neurons are likely to be generally useful as cellular models of these disorders. Future studies may also utilize available human ES cells to investigate species differences. Accordingly, there exists a need for improved cellular/neuronal models of PD and other neurodegenerative disorders.

### SUMMARY OF THE INVENTION

The invention provides for a method for promoting differentiation of a stem cell to a dopamine-producing neuron, comprising co-expressing Nurr1 and PitX3 in the stem cell, thereby increasing the responsiveness of the stem cell to one or more differentiation-inducing stimuli.

The invention further provides for a method for promoting maturation or survival of a dopamine-producing neuron differentiated from a stem cell, comprising co-expressing Nurr1 and PitX3 in the stem cell, thereby increasing the responsiveness of the stem cell to one or more differentiation-inducing stimuli.

In some embodiments of the invention, the stem cell is an adult stem cell or somatic stem cell. In other embodiments, the stem cell is an embryonic stem cell. In yet other embodiments, the stem cell is a mammalian stem cell. In other embodiments, the stem cell is a murine stem cell or a human stem cell.

In another aspect, the invention provides for a method for promoting differentiation of an embryonic stem cell to a dopamine-producing neuron, comprising co-expressing Nurr1 and PitX3 in the embryonic stem cell, thereby increasing the responsiveness of the embryonic stem cell to one or more differentiation-inducing stimuli.

In another aspect, the invention provides for a method for promoting maturation or survival of a dopamine-producing neuron differentiated from an embryonic stem cell, comprising co-expressing Nurr1 and PitX3 in the embryonic stem cell, thereby increasing the responsiveness of the embryonic stem cell to one or more differentiation-inducing stimuli.

In various embodiments of the invention, the differentiation-inducing stimulus comprises brain-derived neurotrophic factor (BDNF), glial cell-line-derived neurotrophic factor (GDNF), sonic hedgehog (SHH), fibroblast growth factor-8 (FGF-8), jagged-1, neuregulin-1β, or any combination thereof.

The invention further provides for a method for producing a dopamine-producing neuron, comprising: (a) co-expressing Nurr1 and PitX3 in an embryonic stem cell; and (b) contacting the embryonic stem cell with one or more differentiation-inducing stimuli, wherein the embryonic stem cell is capable of differentiating into the dopamine-producing neuron.

In one embodiment, the embryonic stem cell comprises an embryoid body. In another embodiment, the embryoid body is in or around stage 3.

In another aspect, the invention provides for a method for producing a dopamine-producing neuron, comprising (a) co-expressing Nurr1 and PitX3 in an adult stem cell or a somatic stem cell; and (b) contacting the stem cell with one or more differentiation-inducing stimuli, wherein the stem cell is capable of differentiating into the dopamine-producing neuron.

In certain embodiments of the invention, the co-expressing comprises transfecting the stem cell with (i) a vector comprising a nucleotide sequence encoding Nurr1; and (ii) a vector comprising a nucleotide sequence encoding PitX3, wherein Nurr1 and PitX3 are co-expressed in the stem cell under appropriate gene expression conditions. In other embodiments, the co-expressing comprises transfecting the stem cell with a vector comprising (i) a nucleotide sequence encoding Nurr1; and (ii) a nucleotide sequence encoding PitX3, wherein Nurr1 and PitX3 are co-expressed in the stem cell under appropriate gene expression conditions. In one embodiment, the vector comprises a viral vector. In another embodiment, the vector comprises a lentiviral vector.

In one aspect, the invention provides an isolated stem cell co-expressing Nurr1 and PitX3 from one or more nucleic acid expression vectors contained therein, wherein the stem cell is capable of differentiating into a dopamine-producing neuron.

In another aspect, the invention provides an isolated dopamine-producing neuron co-expressing Nurr1 and PitX3 from one or more nucleic acid expression vectors contained therein, wherein the dopamine producing neuron is differentiated from an isolated stem cell.

In yet another aspect, the invention provides for a method for replacing dopamine neurons in a subject with a neuron-associated disorder, the method comprising administering to the subject embryonic stem cells co-expressing Nurr1 and PitX3 from one or more vectors contained therein.

In another aspect, the invention provides for a method for replacing dopamine neurons in a subject with a neuron-associated disorder, the method comprising administering to the subject adult stem cells co-expressing Nurr1 and PitX3 from one or more vectors contained therein.

In yet another aspect, the invention provides for a method for replacing dopamine neurons in a subject with a neuron-associated disorder, the method comprising administering to the subject dopamine neurons differentiated from embryonic stem cells co-expressing Nurr1 and PitX3 from one or more vectors contained therein.

In a further aspect, the invention provides a method for replacing dopamine neurons in a subject with a neuron-associated disorder, the method comprising administering to the subject dopamine neurons differentiated from adult stem cells or somatic stem cells co-expressing Nurr1 and PitX3 from one or more vectors contained therein.

In another aspect, the invention provides a method for treating or preventing a neuron-associated disorder in a subject in need thereof, the method comprising upregulating the expression of Nurr1 and PitX3 in the subject.

In various embodiments of the invention, the neuron-associated disorder comprises a brain tumor, a developmental disorder, a neurodegenerative disease, or a seizure disorder. In other embodiments, the neurodegenerative disease comprises Alzheimer's disease, amyotrophic lateral sclerosis (Lou Gehrig's disease), Binswanger's disease, Huntington's chorea, multiple sclerosis, myasthenia gravis, Parkinson's disease, or Pick's disease. In certain other embodiments, the neuron-associated disorder comprises Parkinson's disease.

Another aspect of the invention provides for a method for identifying whether a test compound is capable of enhancing the differentiation of a stem cell expressing Nurr1 and PitX3 to a dopamine-producing neuron, the method comprising (a) contacting a stem cell expressing Nurr1 and PitX3 with a test compound; and (b) determining whether differentiation of the stem cell in (a) is enhanced compared to the differentiation of a stem cell expressing Nurr1 and PitX3 in the absence of the test compound, so as to identify whether the test compound is capable of enhancing the differentiation of the stem cell to a dopamine-producing neuron.

An additional aspect of the invention provides a method for identifying whether a test compound is capable of upregulating Nurr1 and PitX3 activity in a stem cell, the method comprising (a) contacting a stem cell with a test compound; and (b) determining whether activity of Nurr1 and PitX3 are upregulated in the stem cell of (a) compared the activity of Nurr1 and PitX3 in a stem cell in the absence of the test compound, so as to identify whether the test compound is capable of upregulating the activity of Nurr1 and PitX3 in the stem cell.

A further aspect of the invention provides for a transgenic non-human mammal whose somatic and germ cells comprise (a) a DNA segment comprising a nucleotide sequence encoding a Nurr1; and (b) a DNA segment comprising a nucleotide sequence encoding a PitX3, wherein the DNA segments are capable of producing Nurr1 and PitX3 under appropriate expression conditions in the transgenic non-human mammal.

An aspect of the invention also provides for a transgenic non-human mammal whose somatic and germ cells are deficient in one or more genes encoding Nurr1 and PitX3, wherein the transgenic non-human mammal has diminished expression of Nurr1 and PitX3 compared to a wildtype counterpart of the transgenic non-human mammal.

### BRIEF DESCRIPTION OF THE DRAWINGS

FIG. 1. Embryonic dopamine neuron specification. An early transcription factor network (Pax2, Pax5, Otx2) defines the midbrain-hindbrain boundary. Subsequently secreted factors including Sonic Hedgehog (SHH) and FGF-8 define the ventral location of midbrain dopamine neuron precursors. F, forebrain; M, midbrain; H, hindbrain. Fp, floorplate; is, isthmus.

FIG. 2. Both intrinsic factor (left panel) and extrinsic factor (right panel) networks are thought to specify midbrain dopamine neurons. See text for details.

FIG. 3. The generation of ‘marked’ mature midbrain dopamine neurons.

FIG. 4. DY1×Rosa26-lox-stop-lox-LacZ mice display specific marker expression in the substantia nigra but not elsewhere in the CNS. LacZ (blue) and TH (brown) double staining of SN sections. Control single transgenic Rosa26 mice (A) and DY1×LacZ double transgenic animals (B, C). The SN is outlined in (C).

FIG. 5. Stromal cell derived activity-mediated differentiation of DY1 ES cells. DAT immunoreactivity (red) and YFP fluorescence (green) are shown. Most YFP-positive cells display eYFP expression.

FIG. 6. Embryoid body (EB) differentiation of DY1 ES cells. LIF, leukemia inhibiting factor; ITSF, media supplemented with insulin, transferrin, selinium; bFGF, basic fibroblast growth factor; AA, ascorbic acid; div, days in vitro.

FIG. 7. Dopamine uptake activity. (A) Time course of differentiation of DY1 ES cells with SDIA as measured by dopamine uptake. (B) Dopamine uptake activity of DY1 cultures transduced with lentiviral vectors at day 12 of SDIA differentiation.

FIG. 8. Real-time quantitative rt-PCR analysis of dopamine neuron development. (A-B) Real-time PCR analyses for genes specific to midbrain development. Each gene expression value was normalized to that of β-actin and expressed relative to the respective value of the stage 6 DIV GFP control-ES culture. See text for details.

FIG. 9. Replication-defective lentiviral vectors. (A) Single- and (B) two gene-vectors were assembled. LTR, viral long terminal repeat; cPPT, central polypurine tract; CTS, central terminal sequence; EF1α, EF1α promoter region.

FIG. 10. Lentiviral transduction of PitX3, Nurr1, and other transcription factors modifies SDIA differentiation of DY1 ES cells into DNs. DN differentiation was quantified by eYFP (A) fluorescence or TH immunoreactivity (B). Differentiation of serotonergic (C) and GABAergic (C) neurons was also quantified. All results were analyzed by ANOVA. Data represent the mean±SEM. The level of significance is indicated where * p≦0.05 and ** p≦0.005.

FIG. 11. Effect of different soluble factors on the differentiation of DY1 into dopamine neurons with the EB method. (A) eYFP fluorescence or (B) DAT immunoreactivity was quantified by fluorescent confocal microscopy, see text for details. Data represent the mean±SEM. All results were analyzed by ANOVA. The level of significance is indicated where * p≦0.05 and ** p≦0.005.

FIG. 12. DJ-1 Deficient ES Cells are Sensitized to Oxidative Stress. (A) Schematic map of the murine DJ-1 gene in clone F063A04. The retroviral insertion places the engrailed-2 (En2) splice acceptor and the β-galactosidase/neomycin resistance gene fusion (β-geo) between exons 6 and 7. (B) Southern blot analysis of KpnI-digested genomic DNA from DJ-1 homozygous mutant (insertion; −/−), heterozygous (+/−), and wild-type (WT; +/+) cells, probed with murine DJ-1 cDNA. WT DNA shows a predicted 14-kb band, whereas the mutant allele migrates as a 9-kb band. (C) Western blot (WB) of ES cell lysates from wild type, DJ-1 heterozygous and DJ-1 homozygous clones with antibodies to murine DJ-1 or β-actin. DJ-1 migrates at 20 kDa, β-actin at 45 kDa. (D) ES cells were exposed to H₂O₂ for 15 hours and viability was assayed by MTT. DJ-1 heterozygous cells (diamond) and DJ-1 deficient clones 9 (open circle), 16 (solid circle), 23 (square), and 32 (triangle) exposed to H₂O₂. (E-F) Cell death of DJ-1 heterozygous and DJ-1 deficient cells (clone 32) after exposure to H₂O₂ (10 μM) was quantified by staining with propidium iodide and an antibody to Annexin V with subsequent flow cytometric analysis. (G) DJ-1 heterozygous and deficient (clone 32) cells were assayed for apoptosis at 6 and 24 hours after treatment with 10 μM H₂O₂ by Western blotting for cleaved PARP (89 kDa). Data represent means±SEM and were analyzed by ANOVA with Fisher's post-hoc test. *, p≦0.05; **, p≦0.01; ***, p≦0.0001.

FIG. 13. Specificity and Mechanism of Altered Toxin Sensitivity in DJ-1 Deficient Cells. (A-C) Cell viability of DJ-1 heterozygous cells (solid bar) and DJ-1 deficient cells (clone 32; open bar) after 15 hr exposure to H₂O₂, lactacystin, or tunicamycin as assayed by MTT reduction. (D) DJ-1 deficient cells (clone 32) were transiently transfected with a wild-type human DJ-1 vector (solid bar), PD-associated L166P mutant DJ-1 vector (grey bar) or vector alone (open bar). 48 hours after transfection, cells were exposed to 10 μM H₂O₂ for 15 hours and then assayed by MTT reduction. Wild-type human DJ-1 significantly ‘rescued’ survival of the knockout cells, whereas the L166P mutant did not. Similar results were obtained at 20 μM H₂O₂ and with a second DJ-1 deficient clone. Transfection efficiency exceeded 90% in all cases and protein expression level was comparable for human wild-type and L166P mutant DJ-1 as determined by Western blotting. (E) DJ-1 deficient cells (clone 32; open bar) and control heterozygous cells (solid bar) were assayed for intracellular formation of ROS in response to H₂O₂ treatment (15 min, 1 or 10 μM) using Dihydrorhodamine-123 (DHR) and FACS analysis. (F) Protein carbonyl levels were measured by spectrophotometric analysis of DNP-conjugated lysates from DJ-1 deficient (clone 32, solid red line) and control heterozygous cells (dashed blue line). Data are shown as the mean±SEM and were analyzed by ANOVA with Fisher's post-hoc test. *, p≦0.05.

FIG. 14. DJ-1 Deficient ES Cultures Display Reduced Dopamine Neuron Production. (A) The SDIA coculture method. ES cells are cocultured with mouse stromal cells (MS5) in the absence of serum and LIF for 18 days in vitro (DIV). (B) Dopamine neuron production was quantified at 18 DIV by [³H] dopamine uptake assay. DJ-1 deficient ES cultures were defective relative to heterozygous control cultures. (C-D) Neuron production was quantified by immunohistochemical analysis as a percent of Tuj1-positive colonies that express tyrosine hydroxylase (TH) or GAB A. Quantification of TH and GABA immunostaining was performed on all colonies in each of three independent wells. Colonies were scored as positive if any immunostained cells were present. (E) The absolute number of Tuj1 positive colonies was not significantly different among the two genotypes. (F) Kinetic analysis of dopamine neuron differentiation in DJ-1 deficient cultures (clone 32, solid square) and heterozygous controls (open circle) as quantified by dopamine uptake assay. (G) DJ-1 deficient (open bar) and heterozygous control (closed bar) cultures differentiated for 9 DIV and then exposed to 6-hydroxydopamine (6-OHDA) at the indicated dose for 72 hours. Dopamine neurons were quantified by dopamine uptake assay. Data represent the means±SEM and were analyzed by ANOVA followed by Fisher's post-hoc test. *, p≦0.05.

FIG. 15. Neuronal Differentiation of DJ-1-deficient and Control Heterozygous ES Cultures. (A-L) DJ-1 heterozygous (+/−, A-F) and deficient (−/− [clone 32], G-L) cultures were differentiated by SDIA for 18 DIV and immunostained with antibodies for tyrosine hydroxylase (green) and TuJI (red). (A′-L′) Immunostaining of DJ-1 heterozygous (+/−, A′-F′) and deficient (−/−, G′-L′) cultures with antibodies for GABA (green) and TuJI (red). Scale bar, 50 μM.

FIG. 16. RNAi ‘knockdown’ of DJ-1 in primary embryonic midbrain dopamine neurons in primary midbrain cultures display increased sensitivity to oxidative stress. (A-P) Primary midbrain cultures from E13.5 embryos were infected with lentiviral vectors encoding DJ-1 shRNA (or vector alone) under the regulation of the U6 promoter (I-P) or control vector (A-H). Cells were cultured for 1 week after infection and then exposed to H₂O₂ (5 μM) for 24 h. Cultures were immunostained for tyrosine hydroxylase (TH; B, F, J, and N) or dopamine transporter (DAT; C, G, K, or O) and visualized by confocal microscopy. Scale bar, 100 μM. (Q) Cell lysates prepared from midbrain primary cultures infected with DJ-1 shRNA lentivirus (or control vector) were analyzed by Western blotting for murine DJ-1 or β-actin. (R-T) Quantification of TH, DAT, and GFP signal was performed on 10 randomly selected fields in each of three wells for each condition. Red triangles, DJ-1 shRNA treated; Black circles, control vector. Data represent the means±SEM and were analyzed by ANOVA followed by Fisher's post-hoc test. *, p≦0.05.

FIG. 17. Analysis of DJ-1 Deficient ES Cells. (A-B) Cell viability of DJ-1 heterozygous cells (solid bar) and DJ-1 deficient clone 32 (open bar) after exposure to CuCl₂ or staurosporine at the doses indicated. (C) MTT values of untreated DJ-1 deficient ES cell clones and the control heterozygous cells. Assays were performed exactly as in FIG. 12, but in the absence of toxin. (D) MTT values of untreated DJ-1 deficient ES cells transfected with vector alone or various DJ-1 encoding plasmids. Transfection and expression of WT DJ-1 or mutant forms of DJ-1 does not alter the basal metabolic activity or viability of the cells. (E) Western blotting of extracts from ES cells transfected with vectors harboring wild-type human DJ-1 or the L166P mutant, as in FIG. 12.

FIG. 18. Quantitative real-time PCR for DJ-1 gene expression. (A) Real-time PCR analyses of DJ-1 cDNA in wild-type (+/+), heterozygous (+/−), and knockout (−/−) cultures. Each expression value was normalized to that of β-actin and expressed relative to the respective value of the WT (+/+) control. These gene expression patterns were replicated in at least 3 independent PCR experiments. Total RNA from ES cells differentiated with the SDIA method for 18 days was isolated using the Absolutely RNA Miniprep kit (Stratagene). CDNA was synthesized using the SuperScript first strand synthesis system for RT-PCR (Invitrogen). Real-time PCR reactions were optimized to determine the linear amplification range. Quantitative real-time RT-PCRs were performed (Stratagene MX3000P) using the QuantiTect SYBR Green PCR Master Mix (Qiagen) according to the manufacturer's instructions. DJ-1 primer sequences were 5′-CGAAGAAATTCGATGGCTTCCAAAAGAGCTCTGGT (SEQ ID NO:1) and 5′-CAGACTCGAGCTGCTTCACATA CTACTGCTGAGGT (SEQ ID NO:2); primers used for β-actin were 5′-TTTTGGATGCAAGGTCACAA (SEQ ID NO:3) and 5′-CTCCACAATGGCTAGTGCAA (SEQ ID NO:4). For quantitative analyses, PCR product levels were measured in real time during the annealing step, and values were normalized to those of β-actin. (B) Ethidium bromide staining of PCR products obtained after 29 cycles for DJ-1 (625 bp) and β-actin (350 bp).

FIG. 19. Immunocytochemistry for HB9 and GABA neurons in DJ-1 deficient and control heterozygous ES cultures differentiated by SDIA for 18 divisions. Cells were fixed with 4% paraformaldehyde and were stained with rabbit polyclonal antibodies against GABA (Sigma, dilution 1:1000) and mouse monoclonal antibodies against HB9 (dilution 1/50) as in FIG. 16. Scale bar, 50 μM.

FIG. 20. Embryoid body differentiation of DY-1 cells to mDNs. (A) EB differentiation of DY-1 cultures in vitro gives rise to DNs that co-express eYFP and DAT. Bar scale, 50 μm. (B) General scheme of the EB protocol and the expression profiles for Nurr1, PitX3, TH and DAT over the time course of the EB protocol, as analyzed by quantitative real-time rtPCR of cDNA. Data are presented as fold induction (over levels at Stage 2) after normalization for cDNA content (quantified by β-actin levels).

FIG. 21. Nurr1 and PitX3 coordinately specify the ‘late’ maturation of ES-derived mDNs. (A) EB-differentiated DY-1 cells were transduced with lentiviral vectors for control vector (DsRed), Nurr1 alone, PitX3 alone, or Nurr1 and PitX3 vectors. Cultures were subsequently fixed and immunostained with antibodies for eYFP and TH; DsRed2 was visualized by confocal microscopy. Scale bar, 50 μm. (B) mDNs from EB-differentiated DY-1 ES cultures were immunostained for eYFP, DAT, TuJ1, Serotonin (5-HT), or GABA. Cell numbers were quantified in 10 random fields for each sample by confocal microscopy. Data are presented as mean±s.e.m. and were analyzed by Fischer test (ANOVA). * p<0.05 ; ** p<0.005.

FIG. 22. Nurr1 and PitX3 play functionally distinct and synergistic roles in mDN maturation. (A-C, E) Quantitative real-time rtPCR analysis of mDN markers as indicated in EB-differentiated DY-1 (A, B, E) or MM13 (C) ES cells transduced with control vector, Nurr1, PitX3, or Nurr1 and PitX3. (A) Expression levels of Nurr1 and PitX3 are dramatically increased by the respective viral vectors, as predicted. (B,C) Nurr1 overexpression is sufficient to induce early markers such as TH, VMAT-2 whereas Nurr1 and PitX3 coordinately induce the late marker DAT. (D) HPLC quantification of dopamine release by EB-differentiated MM13 ES cells after depolarization with a high potassium media (56 mM KCl) for 15 min. PitX3 alone or Nurr1 together with PitX3 significantly induced dopamine release. Data are shown as mean±s.e.m. and were analyzed by Fischer test (ANOVA); * p<0.05. (E) Markers for other neuron types such as GABAergic (GAD), and serotonergic neurons (TPH and SERT) are weakly induced by Nurr1 but not by PitX3 or the combination, as determined by quantitative real-time rtPCR. Real-time rtPCR levels are presented as a percentage of the value obtained double (Nurr1 and PitX3) transduction after normalization with β-actin.

FIG. 23. PitX3 and Nurr1 coordinately regulate extrinsic factor signaling pathways in EB-differentiated ES cultures. (A) Overexpression of Nurr1 alone is sufficient to induce c-Ret, whereas Nurr1 along with PitX3 additionally induce expression of GFR1α, GDNF, and BDNF in MM13 cultures. (B) PitX3 and Nurr1 co-overexpression expands cells that are sensitive to BDNF and GDNF treatment as quantified by rtPCR for the late marker DAT but not the early marker TH. BDNF and GDNF treatment fails to induce expression of Nurr1 and PitX3. Virally transduced MM13 cells were treated with GDNF and BDNF (both 10 ng/ml, from R&D system) during EB stage 4; real-time rtPCR levels are presented as a percentage of the value obtained double (Nurr1 and PitX3) transduction after normalization with β-actin.

FIG. 24. Functional analysis of Nurr1 and PitX3 transduced, EB-differentiated mDNs in vivo. EB-differentiated cultures (MM13 Stage 3) transduced with either vector alone (n=4) or Nurr1 and PitX3 (n=5) were transplanted by stereotaxic injection into the striatum of adult mice two weeks after unilateral 6-OHDA lesioning. Engraftment was assessed by immunostaining of striatal sections with TH antibody. (A-B) The site of engraftment was identified (white circle) at low magnification; TH immunoreactivity was visualized by confocal microscopy in random fields (10 images per mouse, 20×) within 0.6 mm of the injection site. Quantification of imaging was performed by a blinded observer using NIH Image software (Martinat et al., 2004). Data represent mean±s.e.m. and were analyzed by Fischer test (ANOVA). * p<0.05. (C) Mice transplanted with the Nurr1 and PitX3-transduced cells displayed significantly fewer contralateral rotations in response to apomorphine (0.4 mg/kg) than vector-transduced cells. Mice were habituated to the environment for 20 min prior to the apomorphine injection (0.1 or 0.4 mg/kg); contralateral rotatory behavior was assessed in hemispheric bowls over a 7 min period by a blinded observer. One animal that had been transplanted with control vector-transduced cells displayed severe contralateral barrel rotations in response to 0.4 mg/kg apomorphine) that could not be accurately quantified in this assay and this animal was therefore not included in the statistical analysis. Data were statistically analyzed by the Mann-Whitney test; *, P<0.05.

FIG. 25. Lmx1b plays a role the early differentiation of ES-derived mDNs but does not specify the ‘late’ maturation of ES-derived mDNs. (A) Quantitative real-time rtPCR analysis of mDN markers after transduction of MM13 ES cells with vector alone, Nurr1, Lmx1b or PitX3. Real-time rtPCR levels are presented as a percentage of the value obtained double (Nurr1 and PitX3) transduction after normalization with β-actin. (B) Quantification of dopamine release by EB-differentiated MM13 ES cells after depolarization with a high potassium media (56 mM KCl) for 15 min. Data are shown as mean±s.e.m.

FIG. 26. Effect of PitX3 or Nurr1 knock-down on the expression of TH and DAT.

FIG. 27. (A) Lentiviral transduction of DY-1 cells with vectors harboring either Nurr1 or PitX3 led to persistent overexpression throughout 95% of cells. (B) Analysis of endogenous Nurr1 and PitX3 gene expression in EB differentiated DY-1 cultures indicated that there does not appear to be significant cross-talk between these pathways, as Nurr1 transduction failed to induce PitX3 expression and vice versa. (C) Overexpression of Nurr1 and PitX3 (or both) failed to induce dopamine neuron markers in undifferentiated DY-1 cultures.

FIG. 28. Immunostaining for endogenous markers in an unrelated ES cell line, MM13, shows that the effects of Nurr1 and PitX3 are not a consequence of the DY-1 transgene.

FIG. 29. Nurr1 and PitX3 coordinately induce the maturation of ES cell-derived mDNs. a, Quantitative real-time rtPCR (qPCR) analysis of mDN markers TH and DAT in EB-differentiated MM13 ES cells transduced with control vector (□), Nurr1 (▪), PitX3 [symbol] En-1 (▪), Lmx1b [symbol] Nurr1+PitX3 [symbol] Nurr1+En-1 [symbol] Nurr1+Lmx1b [symbol] PitX3+En-1 (▪), or PitX3+Lmx1b [symbol] Similar results were obtained in 3 independent studies. qPCR levels are presented as a percentage of the Nurr1+PitX3 values after normalization with β-actin. b-c, EB-differentiated DY-1 ES cultures were immunostained for eYFP (specific for DAT-positive cells) or TH. Cell numbers were quantified in 10 random fields per sample by confocal microscopy. Data were analyzed by Fischer test (ANOVA) and are presented as mean±s.e.m. * p<0.05 ; ** p<0.005. n, Nurr1; p, PitX3; np, Nurr1+PitX3; c, Control Vector. d, Specificity of Nurr1+PitX3 induction activity. Nurr1+PitX3 coordinately induce late maturation markers such as DAT and tyrosinase-related protein-1 (TyRP-1). Nurr1 overexpression alone is sufficient to induce TH, and PitX3 alone induces AHD2, marker for the lateral substantia nigra population of mDNs. e, Early midbrain marker En-1 is expressed by more than 95% of EB-differentiated MM13 ES cells, as detected by in situ hybridization, but are not further induced by Nurr1+PitX3. Similar results were obtained with Lmx1b in situ analyses. Scale bar, 50 ∝m. f-g, Markers for other neuron types, such as GABAergic (GAD-67) and serotonergic neurons (tryptophan hydroxylase; TPH), are weakly induced by Nurr1 alone, as determined by qPCR (f) and immunohistochemistry (g). In contrast, Nurr1+PitX3 coordinately inhibit the express of non-mDN markers and do not alter expression of the general early neuronal marker, TuJI.

FIG. 30. Synergistic activity of Nurr1+PitX3 in the maturation of human H9 ES cultures. a-c, Human ES cells (H9) were differentiated using the SDIA method and transduced with control vector (GFP; □), Nurr1 alone (▪), PitX3 alone [symbol] or Nurr1+PitX3 [symbol] Expression of vector-derived Nurr1 and PitX3 were quantified to demonstrate efficacy of the lentiviral transduction. Nurr1+PitX3 coordinately induce mDN maturation as quantified by the expression of multiple markers such as DAT and TH using qPCR (a) or immunostaining (b-c)40. (a). Scale bar, 50 μm. Cell numbers were quantified in 10 random fields for each sample by confocal microscopy. Data are presented as mean±s.e.m. and were analyzed by Fischer test (ANOVA). *, P<0.05. d, GABAergic marker glutamic acid decarboxylase-67 (GAD-67) expression is decreased by Nurr1+PitX3 overexpression. e, Dopamine release from SDIA-differentiated hES cells is potentiated by Nurr1+PitX3 overexpression. Dopamine release was quantified by HPLC analysis of extracellular solution after depolarization with elevated potassium media (56 mM KCl) for 15 min. f, SDIA-differentiated, Nurr1+PitX3-transduced cultures display neuronal characteristics including spontaneous action potentials and burst firing activity. Whole cell patch clamp recordings were conducted in current clamp mode.

FIG. 31. Functional rescue of a PD disease model by Human or mouse ES-derived cultures transduced with Nurr1+PitX3. Adult mice were injected with 6-OHDA unilaterally into the striatum to lesion the endogenous dopaminergic nigrostriatal pathway. After 2 weeks, these mice were transplanted with either human or mouse ES cultures that have been differentiated to the neural precursor stage and transduced with Nurr1/PitX3/GFP lentiviral vectors (n=5 mouse ES cells, n=3 human ES cells) or control GFP alone (mouse ES cells n=4, human ES cells n=3). (a) Mice transplanted with human or mouse Nurr1+PitX3-transduced ES cultures displayed significantly fewer contralateral rotations in response to apomorphine (0.4 mg/kg) than vector-transduced cells (P<0.05). Mice transplanted with either mouse or human ES cultures appeared to function comparably and the data for these were pooled for statistical analyses. Mice were habituated to the environment for 20 min prior to the apomorphine injection (0.4 mg/kg); rotatory behavior was assessed in hemispheric bowls over a 30 min period by a blinded observer. Data were statistically analyzed by Fisher test (ANOVA). b-f, Engraftment was assessed by immunostaining of striatal sections with an antibody for TH, DAT or for the human-specific nuclear antigen (HsNU) and confocal microscopy. TH+ transplanted human ES-derived cells were observed at increased frequency with Nurr1/PitX3 transduction (d-e) relative to control vector (b-c). Furthermore, TH+ processes were observed in the grafts (e) as well as extending into the surrounding host striatal tissue (f) with Nurr1/PitX3 transduction.

FIG. 32. Nurr1 and PitX3 cooperatively activate transcription of DAT promoter sequences. a, Nurr1+PitX3 cooperatively activate transcription of a luciferase marker transcript under the control of DAT gene regulatory sequences. EB-differentiated ES cells or COS7 cells were transduced with Nurr1+PitX3 vectors and then transfected with luciferase test plasmids. Control vector (□), Nurr1 (▪), PitX3 [symbol] and Nurr1+PitX3 Z,2 Chromatin immunporecipitation assays were performed using an antibody to a FLAG epitope-tag at the amino terminus of Nurr1 on differentiated ES cultures transduced with Nurr1+PitX3 or control. Nurr1 bound to proximal sequences in the DAT and TH promoters, but not the β-actin promoter, in the context of PitX3. Similar results were obtained with a polyclonal antibody to PitX3. c-d, Analysis of murine and human DAT promoter sequences identify adjacent Nurr1 and PitX3 binding sites within the proximal DAT promoter. Nurr1 and PitX3 binding sites are indicated with a blue box and an open box respectively. d, Mutation analysis of the DAT promoter using the luciferase assay. Wild-type but not mutant DAT promoter sequences are induced by Nurr1+PitX3 in Cos7 cells. Asterisks in (c) denote nucleotides that are mutated in the binding sites. e, Nurr and PitX3 bind cooperatively to DAT promoter sequences. EMSA analyses were performed using in vitro-translated reticulocyte lysate protein extracts (lanes 1-8) or nuclear extracts from EB-differentiated ES cultures (lanes 9-14). Nurr1 and PitX3 proteins bound cooperatively to sequences from the human DAT promoter (arrow). Apparent DNA-protein complexes were inhibited by competition with unlabelled wild-type (lanes 5,11 3-fold excess, lanes 6,12 10-fold excess) but not mutant oligonucleotides (lanes 7, 13, 3-fold excess; lanes 8, 14, 10-fold excess).

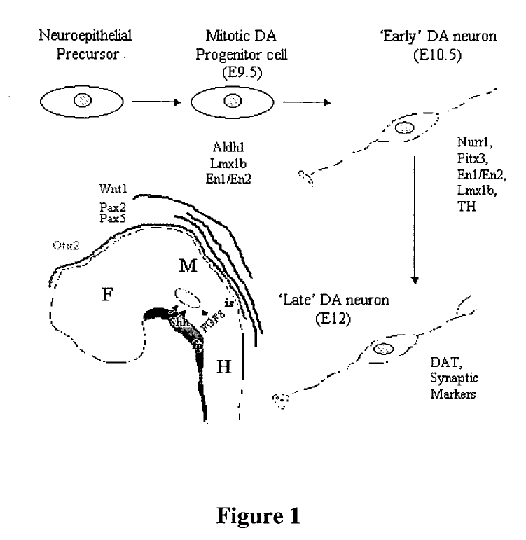

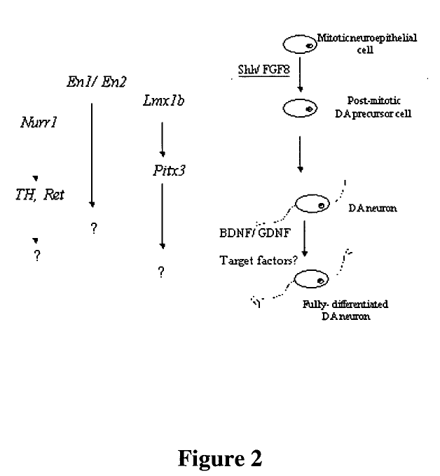

### DETAILED DESCRIPTION OF THE INVENTION

The present invention describes the isolation of distinct cell lines, each of which is useful for analyzing and studying neuron-associated disorders, including brain tumors, developmental disorders, neurodegenerative diseases, and seizure disorders. They are particularly useful for studying and analyzing neurodegenerative diseases, such as Alzheimer's disease, amyotrophic lateral sclerosis (Lou Gehrig's Disease), Binswanger's disease, Huntington's chorea, multiple sclerosis, myasthenia gravis, Pick's disease, and especially Parkinson's disease.

I. Cells Deficient in a Gene Associated with a Neuron-Associated Disorder

The first aspect of the present invention is an isolated cell line derived from mammalian embryonic stem cells, which is deficient in at least one gene associated with the development of a neuron-associated disorder, and methods of isolating such a cell line. Accordingly, one embodiment of the present invention is an isolated cell line derived from mammalian embryonic stem cells, which is deficient in at least one gene selected from the group consisting of DJ-1, wink1 and parkin. In one preferred embodiment, the gene is the DJ-1 gene. In an even more preferred embodiment, the gene is a DJ-1 gene which encodes a protein having a mutation at the cysteine-53 position or the leucine-166 position.

The present invention also describes methods of isolating such a cell line. In one embodiment, the method of the present invention comprises creating a DNA vector, transfecting embryonic stem cells with the DNA vector so that the vector is integrated into the genome of the embryonic stem cells and disrupts the expression of a targeted gene associated with the development of a neuron-associated disorder, and selecting a transfected cell line.

In addition to homologous recombination, other techniques which are able to disrupt a targeted gene's function may also be used. For example, the cell line of the present invention may be generated using gene-trapping technology and RNAi, each of which, either transiently or permanently, disrupts expression of a targeted gene. In one preferred embodiment, the present invention describes a method of creating an isolated cell deficient in at least one gene selected from the group consisting of DJ-1, parkin, and wink1, comprising transfecting embryonic stem cells with RNA interference (RNAi).

Embryonic stem cells and adult or somatic stem cells can be obtained from different organisms. Mammalian stem cells are preferred in the present invention. Human and murine stem cells are even more preferred.

Further according to the present invention, the cell line derived from embryonic stem cells and deficient in a DJ-1 gene, a gene associated with the development of neuron-associated disorders, displays various abnormal phenotypes. For example, these cells display proteasomal inhibition, increased sensitivity to oxidative stress, increased apoptosis, and reduced survival. When treated with toxins, although they appear normal initially, the cells have increased apoptotic cell death due to the accumulation of reactive oxygen species. Accordingly, another embodiment of the present invention comprises methods of identifying toxic compounds that affect the normal development of neurons. In one embodiment, the present invention provides a method of identifying a toxic compound, comprising contacting the cells deficient in a DJ-1 gene with a candidate compound, and determining whether the cells are affected by such contact, for example, by measuring alteration of proteasomal inhibition, level of apoptosis or cell survival.

Using the present invention, a number of compounds have been identified as particularly harmful to cells deficient in the DJ-1 gene. One such compound is H₂O₂. These compounds may be used as references for identifying a toxic compound. Thus, one embodiment of the present invention is a method of identifying a toxic compound that affects the development of neurons, by contacting the cells deficient in a DJ-1 gene with a candidate compound, and comparing the level of proteasomal inhibition, or level of apoptosis or cell survival in such cells as compared to that caused by a known toxic compound.

Most of the toxic compounds that affect the development of neurons are also associated with the development of a neuron-associated disorder. Thus, another embodiment of the present invention comprises methods of identifying one or more toxic compounds that may cause or exacerbate a neuron-associated disorder.

The cell line of the present invention can also be used to identify compounds that promote or enhance the development of neurons. One embodiment of the present invention comprises methods of identifying a compound that promotes or enhances the development of neurons, by determining whether a compound is able to alleviate the oxidative stress displayed by cells deficient in the DJ-1 gene.

By promoting or enhancing the development of neurons, a compound is able to prevent or treat various neuron-associated disorders. Thus, another embodiment of the present invention comprises a method of identifying compounds useful for treating or preventing a neuron-associated disorder, comprising contacting cells deficient in the DJ-1 gene with a candidate compound, and determining whether such a compound is able to alleviate the increased sensitivity to oxidative stress, increased apoptosis level, or reduced survival rate displayed by such cells. One particularly preferred embodiment is a method of identifying a compound useful for treating or preventing Parkinson's disease.

According to the present invention the DJ-1 gene is especially beneficial to neurons and their development. It plays a protective role against oxidative stress and other hazardous conditions. Accordingly, another embodiment of the present invention comprises methods of treating or preventing a neuron-associated disorder in a subject in need thereof, comprising upregulating the activities of the DJ-1 gene in a subject.

There are various methods for upregulating a gene in vivo. For example, a compound capable of upregulating the DJ-1 gene may be administered to a subject in need thereof for treating or preventing a neuron-associated disorder. This compound could promote transcription of the DJ-1 gene, or translation of the protein encoded by the DJ-1 gene. It may prevent degradation of the protein encoded by the DJ-1 gene. Another way of upregulating a DJ-1 gene is to increase the level of transcription factors that regulate the transcription of the DJ-1 gene. This may be achieved by overexpressing one or more transcription factors involved in regulating DJ-1 gene expression. Yet another method of upregulating a DJ-1 gene is to insert an expression promoter into a subject's genome so that this expression promoter is able to enhance the expression of a DJ-1 gene. Yet another method of upregulating a DJ-1 gene is by transiently or constitutively overexpressing an exogenous DJ-1 gene using viral or mammalian expression vectors. It should be noted that there are many approaches to regulating the activities of the DJ-1 gene, and the present invention is not limited to the examples described herein.

By analyzing cells deficient in a DJ-1 gene, the present invention also demonstrates that oxidative stress may be one of the major contributing factors in neuron-associated disorders. Thus, another embodiment of the present invention is a method of preventing or treating a neuron-associated disorder in a subject in need thereof, comprising reducing oxidative stress in the subject. One preferred embodiment comprises a method of treating or preventing Parkinson's disease in a subject in need thereof, by reducing oxidative stress in the subject. Various compounds have been used to reduce oxidative stress, such as free radicals, in a subject. These compounds may be useful for preventing or suppressing neuron-associated disorders, particularly Parkinson's disease. It may also be possible to reduce oxidative stress by upregulating enzymes, such as CAT and SOD, whose function is to eliminate or reduce oxidative stress.

II. Labeled ES Cells and Dopamine Neurons

A second aspect of the present invention is an isolated embryonic stem cell or dopamine neuron capable of expressing at least one detectable label. In one embodiment, the present invention describes undifferentiated embryonic stem cells capable of expressing at least one detectable label. In another embodiment, the present invention describes differentiated embryonic stem cells capable of expressing at least one detectable label. In yet another embodiment, the present invention describes mature dopamine neurons capable of expressing at least one detectable label.

Various detectable labels can be used in the present invention. For example, a label can be a genetic or non-genetic tag. It may also be fluorescent or non-fluorescent. One preferred embodiment of the present invention is an isolated embryonic stem cell or dopamine neuron capable of expressing at least one protein labeled with a fluorescent tag, for example, eYFP. Another preferred embodiment is an isolated embryonic stem cell or dopamine neuron capable of producing at least one protein labeled with β-galactosidase. Yet another preferred embodiment is an isolated embryonic stem cell or dopamine neuron labeled with a chemical agent having high affinity for a dopamine transporter.

The cell line of the present invention may be capable of expressing two or more detectable labels. One preferred embodiment of the present invention is an isolated embryonic stem cell or dopamine neuron capable of expressing two or more detectable labels. An even more preferred embodiment is an isolated embryonic stem cell or dopamine neuron capable of expressing a fluorescent tag and a protein labeled with β-galactosidase.

Cells derived from embryonic stem cells undergo different developmental stages. In one preferred embodiment, the present invention comprises mature dopamine neurons derived from embryonic stem cells, for example, post-mitotic dopamine neurons or neurons that express a dopamine transporter marker. By selecting the loci at which a label may be integrated, the present invention also provides methods of producing stem cells capable of producing at least one detectable label which may be detected at different stages of the differentiation process. For example, one label may be integrated into TH loci, instead of DAT which is a marker specific for mature dopamine neurons.

The availability of such labeled embryonic stem cells and dopamine neurons has a wide range of applications. In one embodiment, the present invention describes methods of detecting the differentiation of embryonic stem cells by measuring the amount of labeled embryonic stem cells. The present invention also describes methods of identifying a compound that affects neuron differentiation by contacting a labeled embryonic stem cell with a candidate compound, and determining whether the candidate compound alters or delays stem cell differentiation by measuring the amount of labeled stem cells.

In addition to identifying compounds, the methods of the present invention may also be used to identify endogenous factors or elements, for example, other genes involved in the differentiation process. One embodiment of the present invention comprises methods of identifying a gene involved in differentiation of stem cells, comprising upregulating or down-regulating a selected gene in embryonic stem cells capable of expressing at least one detectable label, measuring the amount of labeled stem cells, and determining whether such upregulation or downregulation alters or affects stem cell differentiation.

In one preferred embodiment, a gene of interest may be cloned into an expression vector, preferably a mammalian expression vector or a viral vector. The expression vector is used to transfect embryonic stem cells capable of expressing at least one detectable label. Differentiation of the stem cells is determined by measuring the level of detectable label to determine whether the differentiation process is altered or affected by such transfection. In another preferred embodiment, protein encoded by a gene of interest may be obtained in vitro and added to the undifferentiated embryonic stem cells capable of expressing at least one detectable label to determine whether such protein affects or alters the differentiation, maturation, and/or survival of such stem cells.

Many compounds that affect the differentiation of embryonic stem cells are also associated with the development of neuron-associated disorders. Thus, another embodiment of the present invention is a method of identifying a toxic compound, which affects the differentiation of stem cells or the survival of dopamine neurons by determining whether a candidate compound suppresses or prevents differentiation of embryonic stem cells. Similarly, the same method may also be used to determine whether a compound adversely affects dopamine neurons, which are essential for the development of neuron-associated disorders.

The present invention also provides methods of identifying compounds that are useful for preventing or treating neuron-associated disorders, particularly Parkinson's disease, comprising contacting embryonic stem cells or dopamine neurons capable of expressing at least one detectable label with a candidate compound, and detecting whether such contact increases the amount of labeled proteins in such stem cells or dopamine neurons.

The cell line of the present invention may also be used in monitoring and enhancing the efficacy of stem-cell transplantation. Thus, one embodiment of the present invention is a method of increasing the efficacy of stem-cell transplantation in a subject in need thereof, comprising administering to the subject embryonic stem cells capable of producing at least one detectable label, and tracing the labeled protein to determine the efficacy of transplantation. This method is particularly suitable for transplanting undifferentiated embryonic stem cells or stem cells at early stages of differentiation. It is also applicable to transplantation of dopamine neurons.

The present invention further provides a transgenic animal (e.g., a mouse) capable of producing at least one detectable label. In particular, the present invention describes a transgenic animal having dopamine neurons capable of producing at least one detectable label. More preferably, the present invention describes a transgenic animal having dopamine neurons capable of producing fluorescent protein (eYFP), β-galactosidase, or the combination thereof.

In one aspect of the invention, the compound can be combined with a carrier. The term “carrier” is used herein to refer to a pharmaceutically acceptable vehicle for a pharmacologically active agent. The carrier facilitates delivery of the active agent to the target site without terminating the function of the agent. Non-limiting examples of suitable forms of the carrier include solutions, creams, gels, gel emulsions, jellies, pastes, lotions, salves, sprays, ointments, powders, solid admixtures, aerosols, emulsions (e.g., water in oil or oil in water), gel aqueous solutions, aqueous solutions, suspensions, liniments, tinctures, and patches suitable for topical administration.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of ≦20%.

The term “effective” is used herein to indicate that the inhibitor is administered in an amount and at an interval that results in the desired treatment or improvement in the disorder or condition being treated (e.g., an amount effective to modulate the growth of kidney tissue).

In some embodiments, the subject is a human, mouse, rabbit, monkey, rat, bovine, pig, sheep, goat or dog.

Pharmaceutical formulations include those suitable for oral or parenteral (including intramuscular, subcutaneous and intravenous) administration. Forms suitable for parenteral administration also include forms suitable for administration by inhalation or insufflation or for nasal, or topical (including buccal, rectal, vaginal and sublingual) administration. The formulations may, where appropriate, be conveniently presented in discrete unit dosage forms and may be prepared by any of the methods well known in the art of pharmacy. Such methods include the step of bringing into association the active compound with liquid carriers, solid matrices, semi-solid carriers, finely divided solid carriers or combinations thereof, and then, if necessary, shaping the product into the desired delivery system.

The present invention is better understood in light of the following examples, which should not be construed to limit in any way the scope of the invention as defined in the claims which follow thereafter. While the invention will be described herein in some detail, for purposes of clarity and understanding, it will be appreciated by one skilled in the art, from a reading of the disclosure, that various changes in form and detail can be made without departing from the true scope of the invention in the appended claims.

### EXAMPLE 1

### Generation of a ‘Marked’ Reporter ES Cell Line

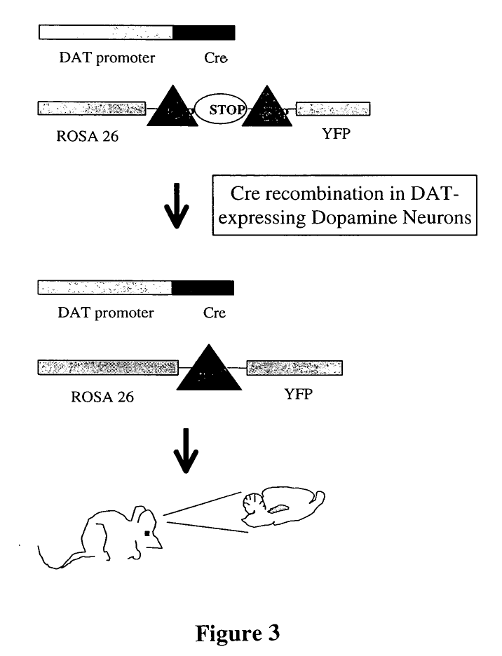

To examine the process by which mouse ES cells acquire a dopaminergic phenotype, murine ES cell lines were produced capable of giving rise to ‘marked’ mature dopamine neurons (DNs) identifiable by the expression of enhanced yellow fluorescent protein (eYFP) or β-galactosidase (LacZ). A Cre-recombinase based 2-transgene approach was used (FIG. 3). This method has been broadly used in whole animals for cell type-specific and tissue-specific expression (Srinivas et al., 2001). Briefly, the phage-derived Cre recombinase was expressed specifically in midbrain dopamine neurons along with a second transgene that harbors a marker gene under the regulation of Cre recombinase. A strain of mice was derived in which Cre recombinase was “knocked-in” at the dopamine transporter (DAT) locus, a ‘late’ marker of dopamine neurons (Zhuang et al., 2001). This marker is more specific than other markers, such as TH, since TH is also expressed in other catecholaminergic cell types such as norepinephrine cells in the locus ceruleus. Furthermore, DAT is expressed at a later developmental point than TH in vivo and in vitro (Barberi et al., 2003).

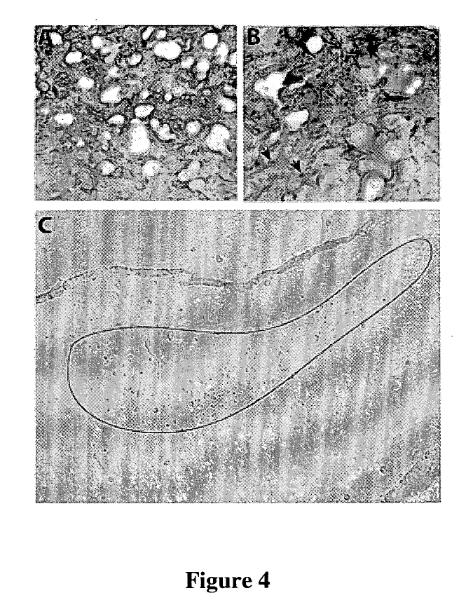

Using the same approach, a second transgenic mouse line was obtained that harbors the eYFP (or LacZ) gene inserted into the constitutively-expressed ROSA26 locus, preceded by loxP-flanked stop sequence (Srinivas et al., 2001); thus, in cells expressing Cre recombinase, Cre-mediated excision of the loxP-flanked transcriptional stop sequence allows for marker gene expression. The double transgenic progeny display expression of marker gene specifically in midbrain DNs (FIGS. 3 and 4)(Staropoli et al., 2003), however, they do not display any significant developmental defects. ES cell lines were derived from double transgenic blastocysts using standard embryological techniques (Wichterle et al., 2002). One double-transgenic ES cell clone, DY1, was demonstrated to be totipotent by injection into blastocysts and the generation of 100% ES-derived chimeric animals with germline transmission (FIG. 4).

### EXAMPLE 2

### Differentiation of DY1 ES Cells into ‘Marked’ Dopamine Neurons

Two established and complementary protocols to differentiate ES cells into DNs have been described. The embryoid body (EB) method (Lee et al., 2000) involves several steps: first, spherical cell aggregates (termed embryoid bodies) are generated that contain ectodermal, mesodermal and endodermal derivatives; second, these aggregates are selected for neuronal precursors and expanded with basic-FGF (bFGF); and third, differentiation is induced by growth factor withdrawal. DN differentiation is observed in vitro in terms of TH expression, an early marker of the dopamine lineage (Chung et al., 2002; Lee et al., 2000). There is also vesicular dopamine release, although this may be at a level that is significantly reduced below that found in primary midbrain cultures (Kim et al., 2002; Kim et al., 2003) (and consistent with our unpublished data).

A second protocol, called Stromal Cell-Derived Inducing Activity (SDIA), is a single step co-culture of ES cells and bone-marrow stromal cells (Kawasaki et al., 2002a). The molecular determinants of SDIA have not been defined but may represent multiple factors necessary for early neural induction as well as dopamine neuron specification. There is evidence for bone morphogenic signal (BMP) inhibition, which is known in vivo to be essential for the early specification of neural progenitors. This method appears to generate a higher percentage of TH-positive cells than the EB method (Barberi et al., 2003) and these cells appear capable of dopamine release in vitro, although (as with EB differentiation) dopamine levels may be at a significantly reduced level compared to primary midbrain cultures (Bagri et al., 2002). Thus, these protocols may be inefficient at generating fully mature neurons in vitro. When transplanted into the striata of unilaterally 6-OHDA lesioned rodents, both EB and SDIA ES-derived DNs appeared to ‘rescue’ the amphetamine or apomorphine-induced turning behavior (Barberi et al., 2003; Kim et al., 2002). These data suggest the possibility that environmental determinants present in the adult CNS may be capable of inducing the terminal differentiation of transplanted dopamine neurons.

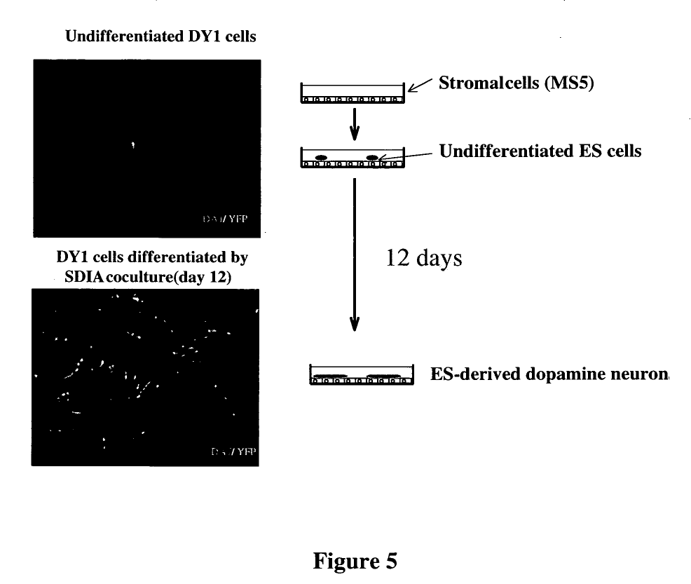

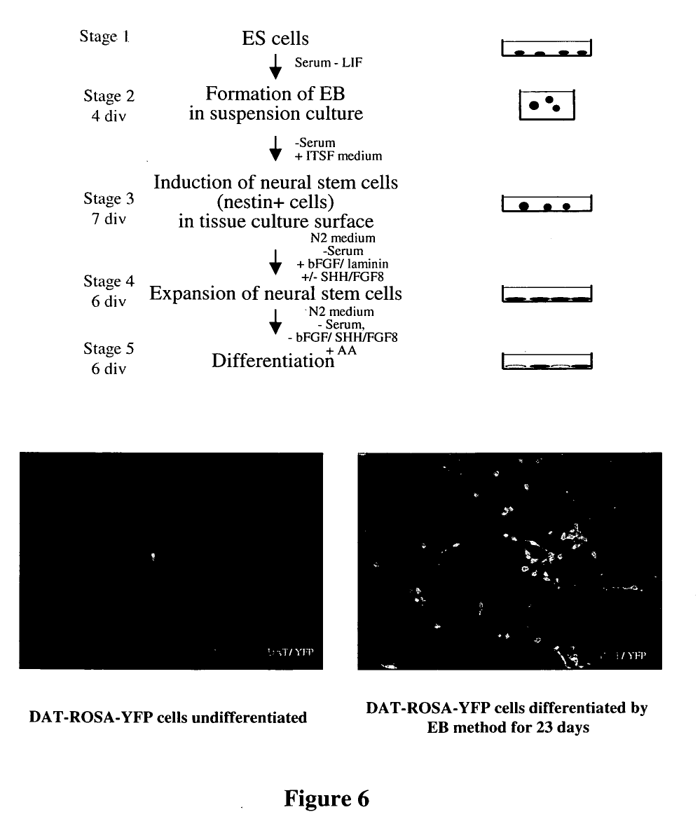

Using each of these two protocols, the DY1 ES cells were differentiated and gave rise to eYFP-positive cells, as shown in FIGS. 5 and 6. In contrast, few eYFP positive cells were detected in non-differentiated cultures. The eYFP positive cells were specifically immunostained with a monoclonal antibody for DAT as shown in FIGS. 5 and 6 and another monoclonal antibody for TH, which confirmed the restricted expression of eYFP to ‘late’ differentiated DNs. Appropriate fluorescence-conjugated secondary antibodies were used in immunostaining as described (Staropoli et al., 2003). Not all TH-immunostained cells were positive for eYFP. These results indicate that the DY1 ES cells were differentiated in vitro into DNs and that these cells were then used to examine the differentiation of ES cells into DNs.

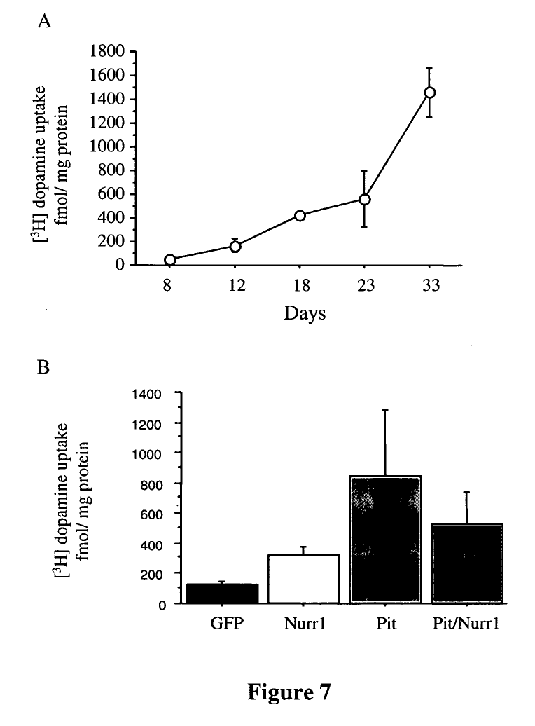

The differentiated state of the ES-derived DNs was further confirmed by quantifying additional dopamine neuron-specific markers and activities. Dopamine transporter activity was measured in terms of the uptake of radioactive dopamine (Johnson et al., 1998). Dopamine uptake activity was found to be present in DY1 cultures differentiated by either the SDIA or EB method, but this activity appeared significantly later than TH and other markers. For example, cells differentiated by SDIA displayed a low level of dopamine uptake activity at day 8, which increased dramatically at day 30 (FIG. 7). In contrast, we detect Nurr1 expression by RT-PCR at day 5, and TH expression as well as PitX3 expression are apparent at days 8 to 18 by quantitative real-time RT-PCR for mRNA. As predicted, dopamine uptake activity in DY1 cells, which are hemizygous at the DAT gene locus, is reduced (relative to D3 wild-type cells).

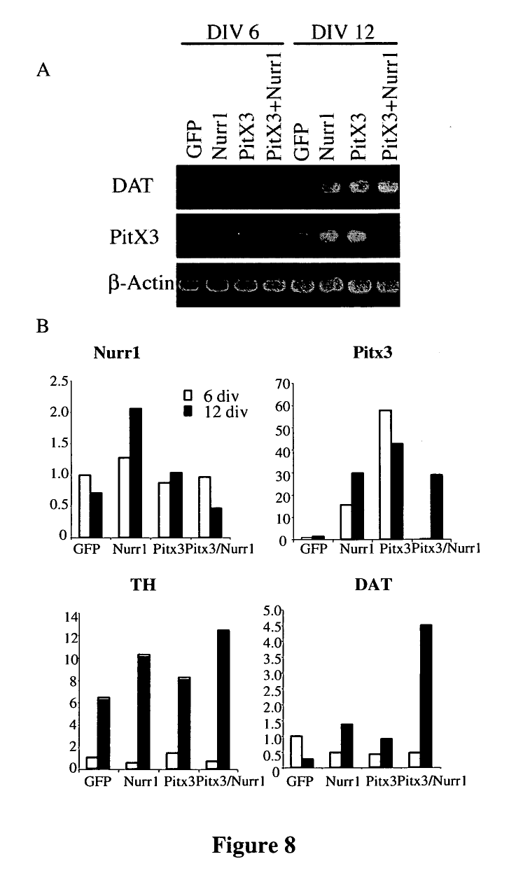

Similarly, we have measured additional markers of the ‘late’ DN phenotype. Depolarization-induced dopamine release as well as total cellular dopamine (as a ratio of total cellular protein) is apparent at later stages of differentiation. For instance, using the SDIA method, dopamine release is apparent at approximately days 12-14 (Barberi et al., 2003) and increases thereafter, in parallel with DAT activity. We have also measured a number of early and late markers of DN differentiation using real-time RT-PCR (FIG. 8). Total RNA was isolated using a standard protocol (Qiagen) from cultures at different time points of SDIA-mediated differentiation, and cDNA was generated (Invitrogen First Strand Kit). Real-time PCR was performed as per the manufacturer's instructions (Cepheid) using oligonucleotides specific for genes that are expressed during the differentiation course. Relative mRNA concentrations were normalized to levels of β-actin as an internal control (Heid et al., 1996). TH first appears at day 8, and PitX3 appears at day 12 and thereafter. DAT expression at the RNA level is observed initially at day 12, consistent with immunohistochemistry and activity assays as described above (FIG. 8). Thus, we have described multiple early and late markers of dopamine neuron differentiation, and these markers allow for a kinetic analysis of events in DN differentiation in vitro.

### EXAMPLE 3

### Lentiviral Vectors

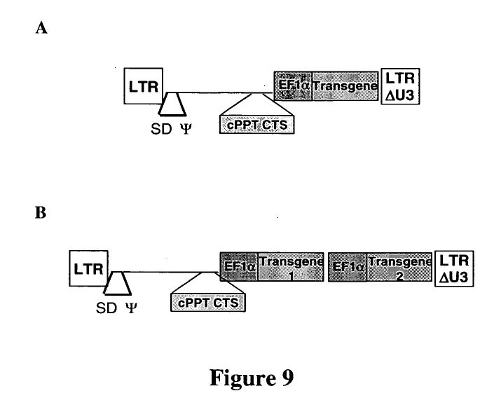

We have generated lentiviral vectors that express human Nurr1 or PitX3 (TFs implicated in dopamine neuron development and selectively expressed in post-mitotic midbrain DNs during development) cDNAs and transduce nearly 100% of cells in an ES culture and allow the overexpression of genes in mitotic or postmitotic cells (Zennou et al., 2001). Expression is induced over 20-fold, as confirmed by real-time quantitative RT-PCR (FIG. 8). Additionally, we have generated vectors that harbor pairs of cDNAs, including PitX3 and Nurr1 together, or either PitX3 or Nurr1 along with a fluorescence marker such as dsRed2 (FIG. 9).

We have also generated Lentilox (Rubinson et al., 2003) RNAi-based vectors that target the expression of Nurr1 and PitX3. Lentilox vectors harbor the U6 promoter to drive the expression of stem-loop sequences that mediate RNAi-based inhibition of target gene expression in order to knock-down the expression of sequences of interest, such as Nurr1 or PitX3 in postmitotic cells. We have knocked down gene expression by 90% in post-mitotic neurons using this technique (as ascertained by protein blotting). All lentiviral vectors are prepared identically and quantified by p24 protein enzyme-linked immunoassay (Zennou et al., 2001).

Finally, we have generated additional viral constructs that are Cre-inducible in that they harbor the identical lox-stop-lox cassette present in the marker transgene as present in the Rosa26 locus of DY1 cells. This allows for the conditional expression of virally transduced genes only in cells of interest that express the Cre recombinase—mature dopamine neurons. Initial analysis of this system using a dsRed marker transgene confirms its efficacy.

### EXAMPLE 4

### PitX3 and Nurr1 Overexpression in ES-Derived DN Generation

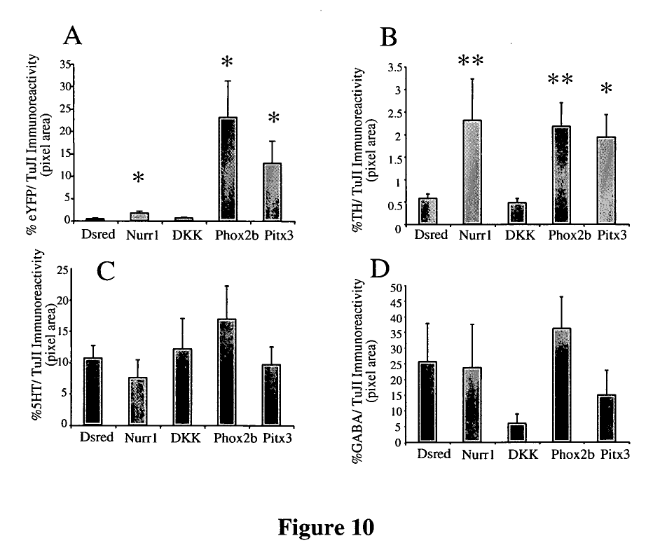

Using the ES cell differentiation assays and lentiviral vectors described in the previous examples, DY1 cells were infected with control (dsRED), Nurr1, or PitX3 vectors 2 days prior to the initiation of differentiation. DN generation was quantified after either SDIA or EB differentiation by eYFP fluorescence or immunohistochemical analyses. We confirmed the activating role of Nurr1 in the context of either differentiation protocol as quantified by eYFP expression of DY1 cells (FIG. 10). Additionally, we observed that PitX3 appears to be effective in this assay. Similar results were obtained by quantitative analysis of TH immunostaining (FIG. 10), whereas the generation of serotonin (5HT) and GABAergic neurons was not significantly altered. Also, dopamine uptake activity appears increased in cells that overexpress Nurr1 or PitX3 (FIG. 7). Real-time rt-PCR analysis shows that Nurr1 and PitX3 overexpression appear to synergize in inducing DAT (FIG. 8), and Nurr1 is effective in inducing PitX3 activity, consistent with an interaction between these pathways.

### EXAMPLE 5

### Additional Transcription Factors

We also investigated the function of two other transcription factors in this pathway using viral overexpression. Phox2b is a transcription factor that plays a necessary and sufficient role in the induction of locus cerulius norepinephrine (NE) neurons, which arise in the dorsal hindbrain and express several markers in common with DNs. For instance, TH is a key enzyme in both cell types, and consistent with this, TH expression is induced efficiently by Phox2b overexpression. Surprisingly, we found that Phox2b also efficiently induces the expression of eYFP in DY1 cells.

### EXAMPLE 6

### Role of Brain Region-Specific Environmental Factors in Coculture

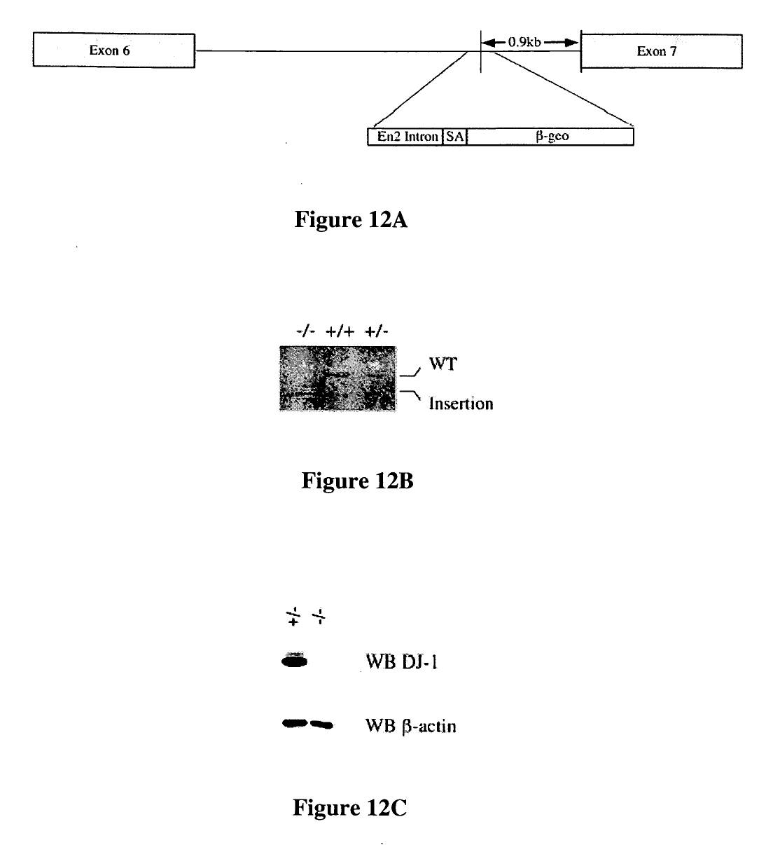

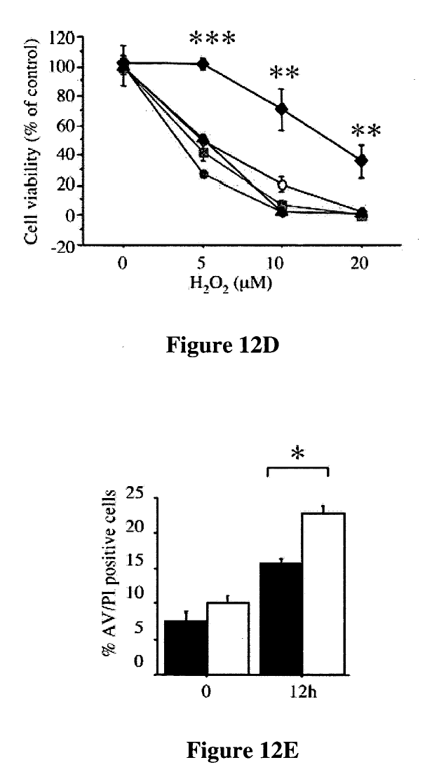

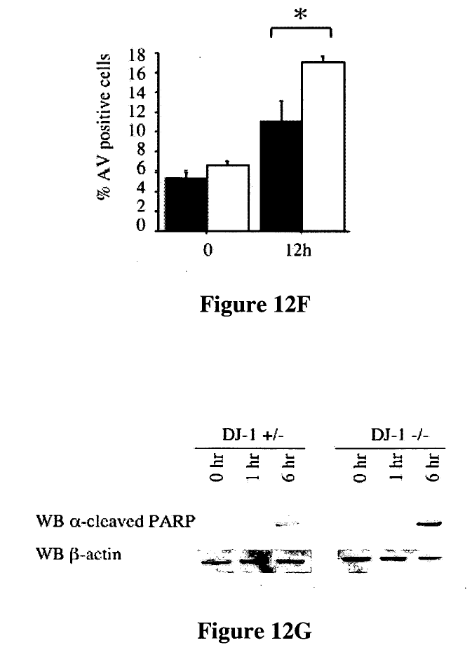

To investigate whether such factors would modify the differentiation of ES-derived DNs, DY1 cells were differentiated using SDIA and subsequently ES cells were isolated using mild enzymatic (dispase) treatment (Kawasaki et al., 2002b) and replated on primary embryonic brain cultures obtained from E17 striatum (Prochiantz et al., 1979), cortex (at day 7 in vitro), or as a control onto another stromal cell layer. Our preliminary results indicate that the generation of DAT-positive cells is increased in the context of primary cultures of striatum or cortex (FIG. 12). These data suggest that striatal or cortical cells stimulate the development of the dopaminergic neurons.

### EXAMPLE 7

### Candidate Soluble Factors

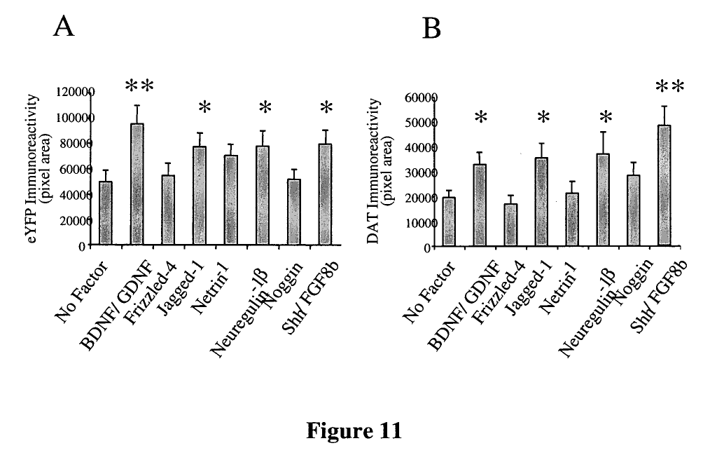

We used marked ES-derived ‘late’ dopamine neurons to analyze candidate soluble and cell-associated factors in the differentiation process. Initially, we focused on previously described factors that have been shown to play a role in early or late events in the process of dopamine neuron differentiation. Briefly, DY1 ES cells were differentiated using EB or SDIA and candidate factors were added to the standard differentiation protocol at stages 4-5 of differentiation (Lee et al., 2000). We found that Sonic Hedgehog and FGF-8 treatment led to a significant increase in either eYFP fluorescence or DAT immunoreactivity (FIG. 11). These data confirm a role for these factors (Lee et al., 2000) and validate the utility of the DY1 assay system.

We have tested additional candidate factors that are implicated in the differentiation of dopamine neurons and other CNS neuron classes. For instance, the Notch pathway plays an inhibitory role in early neuronal fate determination in uncommitted proliferative cells including neuronal stem cells (Bixby et al., 2002), as well as inhibitory roles in later events such as neurite outgrowth (Berezovska et al., 1999) in hippocampal cells. Inhibiting Notch signaling using a soluble receptor Jagged (R&D Systems) protein led to a significant increase in terminally differentiated dopamine neurons. Another multifunctional factor in the early and late determination of neurons is neuregulin, which inhibits neurogenesis early but subsequently plays a role promoting synapse formation at certain CNS and PNS synapses (Buonanno and Fischbach, 2001). Neuregulin1-β1 appears to have increased the propensity of ES cultures to differentiate into DAT-positive cells.

### EXAMPLE 8

### Primary Mesencephalic Cultures

The differentiation of midbrain dopamine neurons in vivo is recapitulated in ES-derived cultures and in primary neuronal cultures. To extend the above analyses of dopamine neuron differentiation, and to obtain an independent assay for ‘late’ events in DN maturation, we have also generated a primary mesencephlic differentiation assay. Cultures are prepared from embryonic day E13.5 CD1 mice (Staropoli et al., 2003). These cultures generate TH and DAT positive neurons over the first 7 days in culture. We have described the use of lentiviral vectors to transduce greater than 95% of cells in these cultures, including primary dopamine neurons (Staropoli et al., 2003).

Also, we have generated primary midbrain cultures from DY1 mice that differentiate into eYFP-positive DNs, thus allowing for the analysis of terminal dopamine neuron differentiation. These cells can be used to perform a detailed kinetic analysis of dopamine neuron generation using real-time imaging techniques.

### EXAMPLE 9

Transplantation of in vitro Generated Dopamine Neurons into Lesioned Mouse Striata

In the above examples, we described a preliminary kinetic analysis of dopamine neuron generation in vitro. A novel aspect of the invention is the ability to focus on ‘late’ events in the differentiation of dopamine neurons. Furthermore, we describe a novel reagent, a fluorescent marker for ‘late’ dopamine neuron differentiation.

We have studied the efficacy of transplantation into the striatum of unilaterally 6-OHDA lesioned animals. The study protocol is essentially as described (Barberi et al., 2003; Morizane et al., 2002). 6-7 week-old male 129/sv mice (18-22 g) were housed and treated according to NIH guidelines. They were anesthetized with sodium pentobarbital (30 mg/kg) and then 0.5 ul 6-OHDA (Sigma-Aldrich; 8 ug/ul in PBS with 0.05% ascorbic acid was injected unilaterally into the striatum at the following coordinates with respect to the bregma: A +1.0, L −2.2, V −3.0 with ear bars at +0.25 using a stereotaxic apparatus for mice (Stoelting). To protect noradrenergic neurons, 30 minutes before the injection desipramine was injected intraperitoneally at a dose of 25 mg/kg.

For transplantation, cells were trypsinized gently and resuspended in N2 media at 50,000 cells/ul. Transplantations were performed under sodium pentathol anesthesia and all surgical and animal care procedures were as according to the NIH and IACUC. Cells were transplanted using a stereotaxic apparatus into the lesioned striatum (from the bregma: A +1.0, L +2.0, V +3.0, incisor bar 0) via a Hamilton microsyringe fitted with a 26-gauge blunt needle. Successful engraftment was assessed using standard immunohistochemical methods at 4 weeks.

### EXAMPLE 10

### Generation of DJ-1 Deficient ES Cells

We generated cells deficient in DJ-1. cDNA for DJ-1 was PCR amplified from human liver cDNA (Clontech) and cloned into the expression vectors pET-28a (Novagen) or pcDNA3.1 (Invitrogen) using standard techniques. Flag-DJ-1 and all described mutants were generated by PCR-mediated mutagenesis. For protein carbonyl quantization (Bian et al. 2003), cells were plated (1.4×105 cells per well), grown for 24 hours, and then treated with 10 mM H₂O₂ as indicated. Cleared lysate (40 ml) from each time point was added to 2 M HCl (120 ml) with or without 10 mM DNPH and incubated for 1 h at 24° C. with shaking. Proteins were then TCA precipitated and resuspended in 200 ml 6M Guanidinium Chloride. Absorbance was measured at 360 nm, and DNP-conjugated samples were normalized for protein concentration with the underivitized control samples. Undifferentiated ES cells were cultured using standard techniques (Abeliovich et al. 2000). SDIA differentiation of ES cultures was performed as described (Kawasaki et al. 2000) except that ES cells were plated at a density of 500 cells/cm² and cocultured with the MS5 mouse stromal cell line (Barberi et al. 2003). Transfections were performed using Lipofectamine 2000 (Life Technologies) for 18-36 hours as per the manufacturer's instructions (Staropoli et al. 2003). Primary cultures and infections were performed as described (Staropoli et al. 2003). A murine embryonic stem (ES) cell clone, F063A04, that harbors a retroviral insertion at the DJ-1 locus was obtained through the German Gene Trap Consortium (http://tikus.gsf.de) (Floss and Wurst 2002). The pT1ATGβgeo gene trap vector is present between exons 6 and 7 of the murine DJ-1 gene, as determined by cDNA sequencing of trapped transcripts and genomic analysis (FIG. 12A). This integration is predicted to disrupt the normal splicing of DJ-1, leading to the generation of a truncated protein that lacks the carboxy-terminal domain required for dimerization and stability. Of note, a mutation that encodes a similarly truncated protein (at the human DJ-1 exon 7 splice acceptor) has been described in a patient with early-onset PD (Hague et al. 2003).

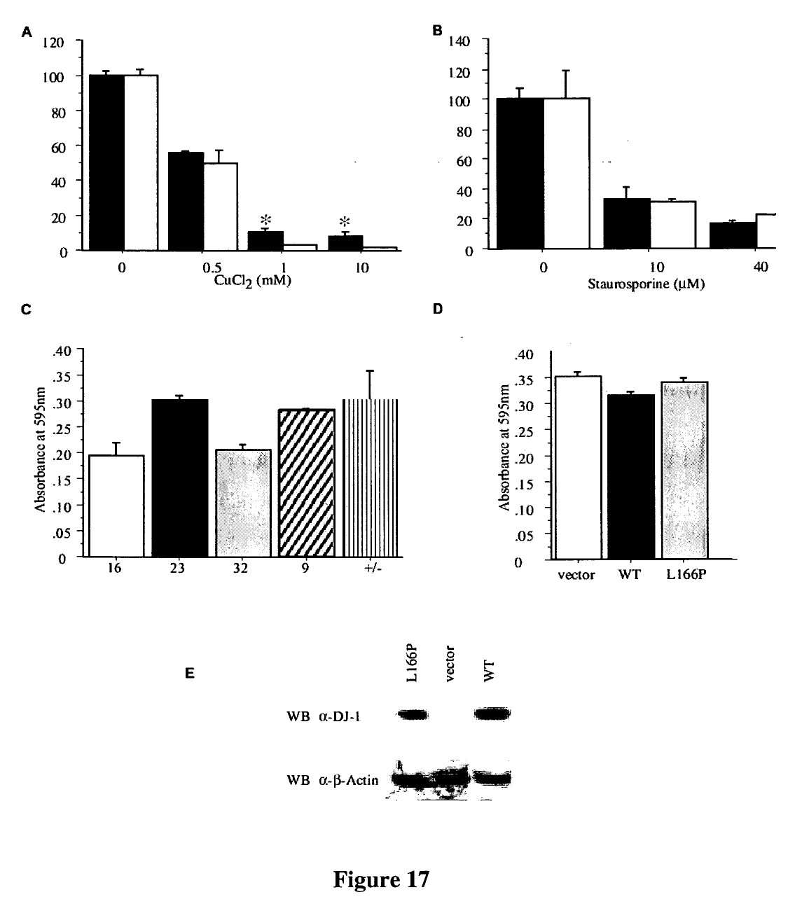

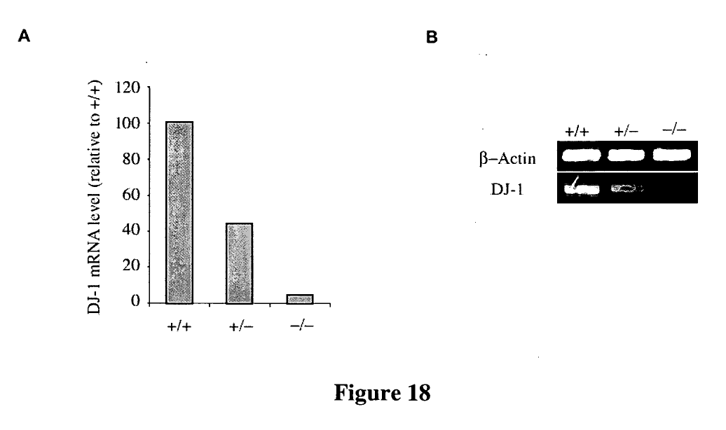

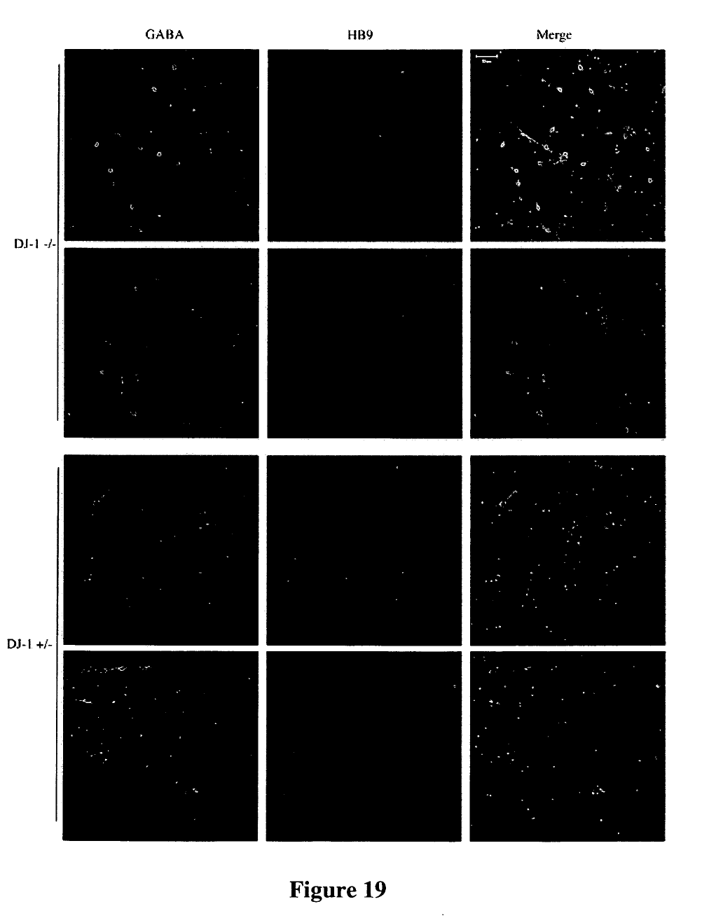

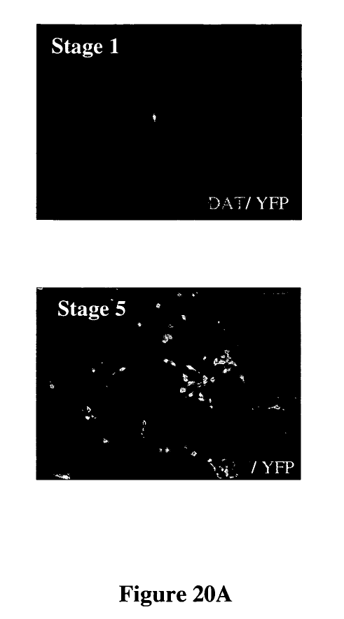

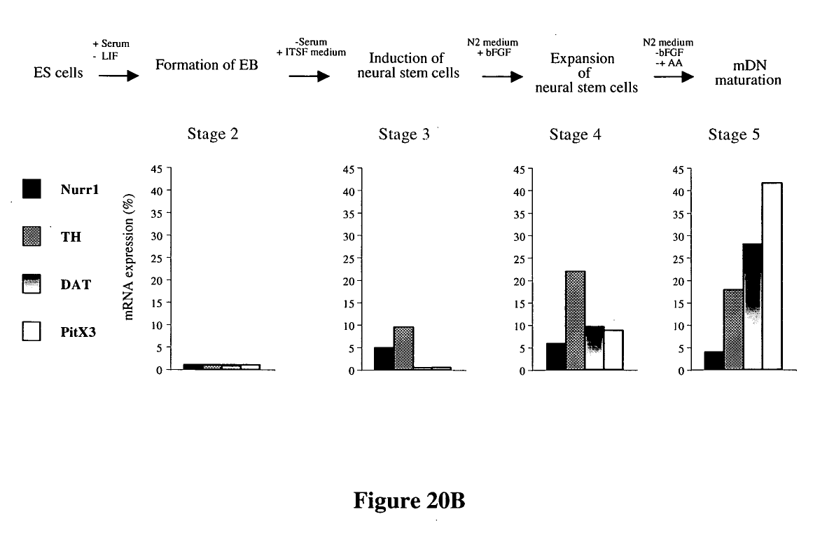

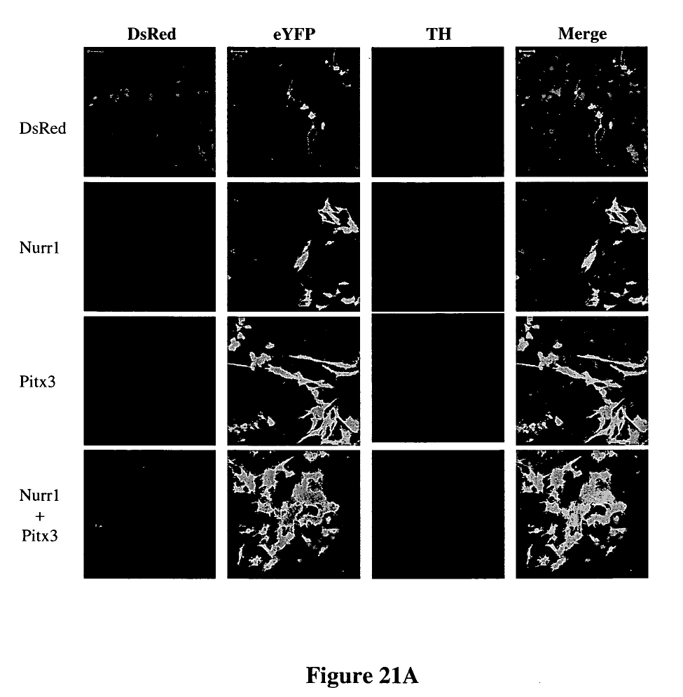

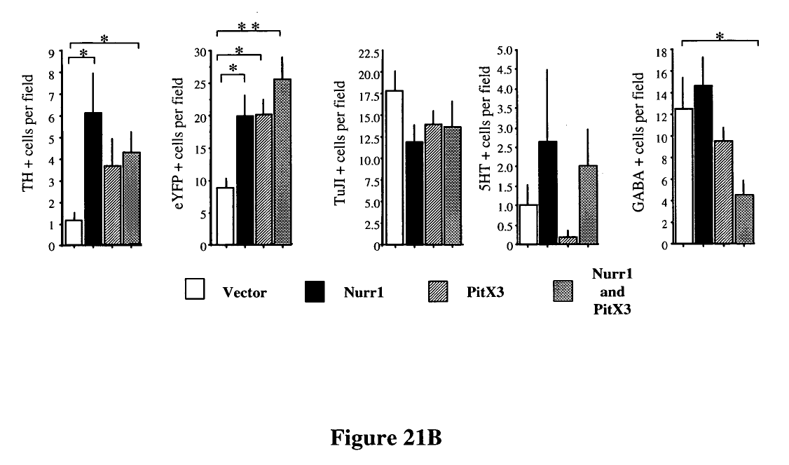

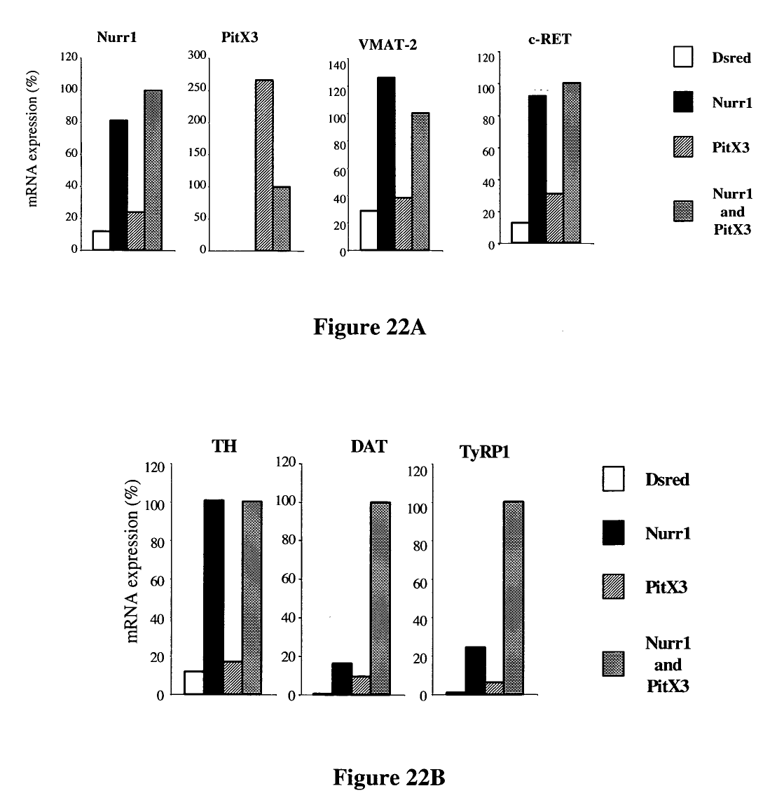

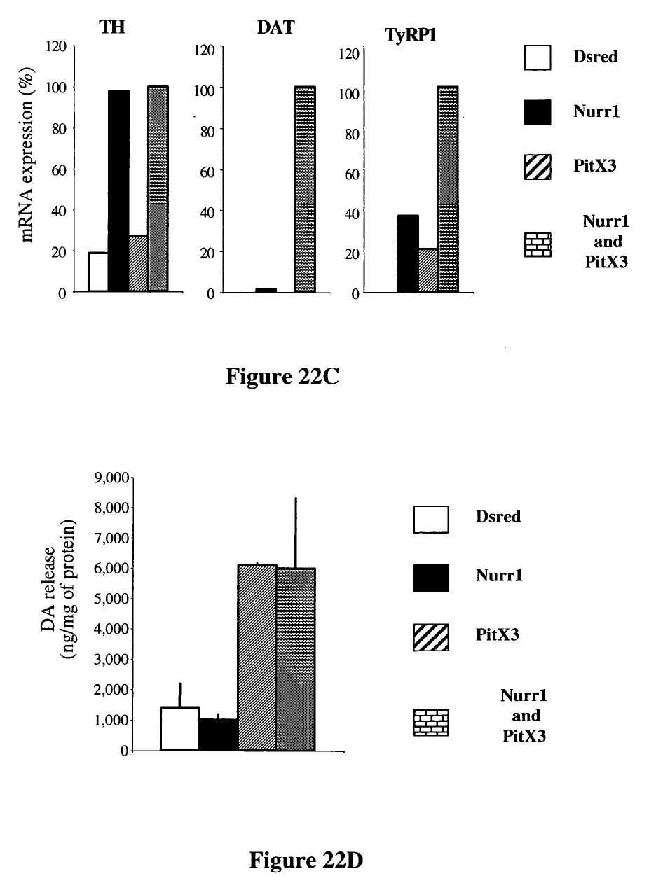

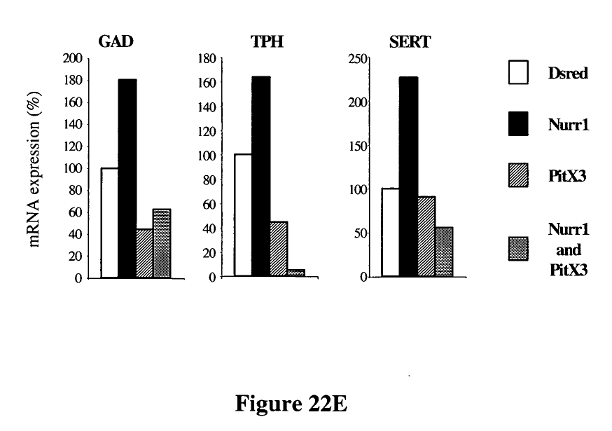

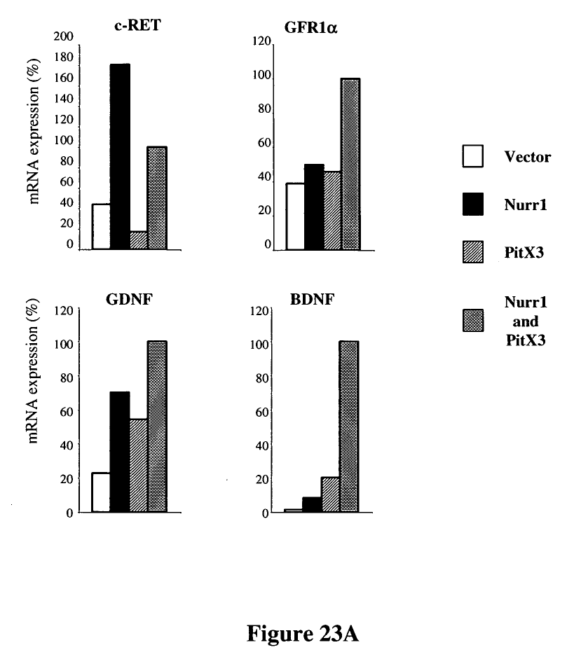

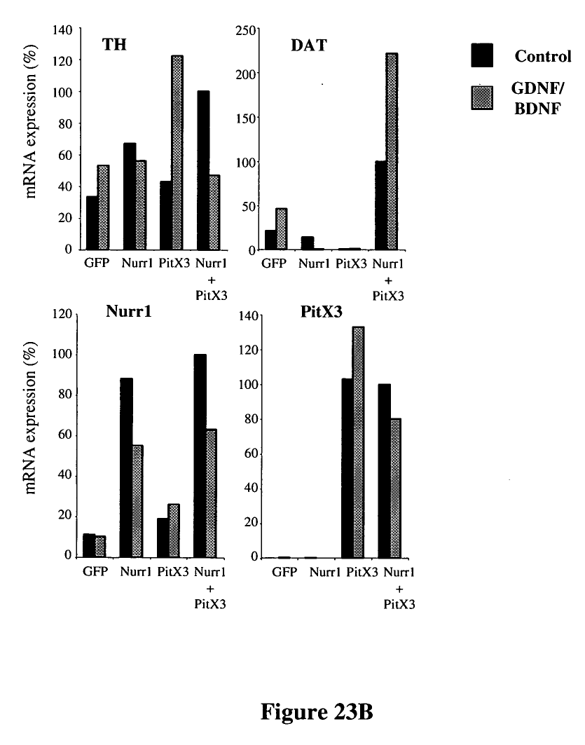

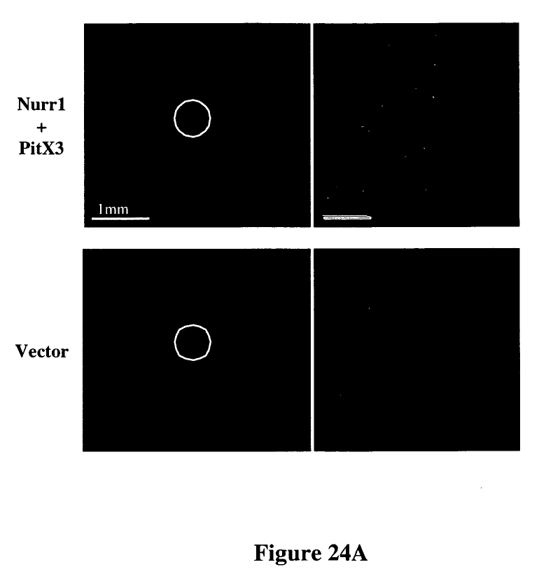

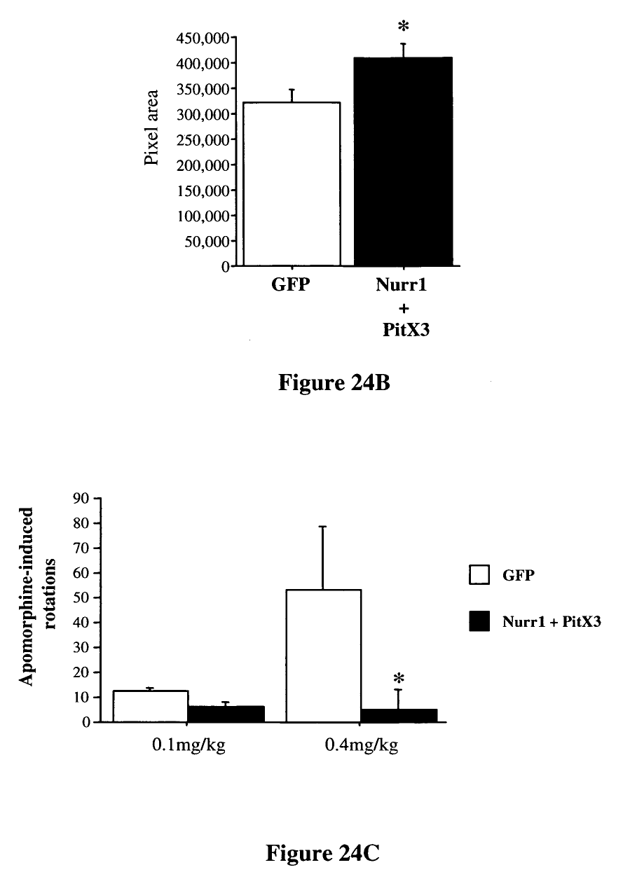

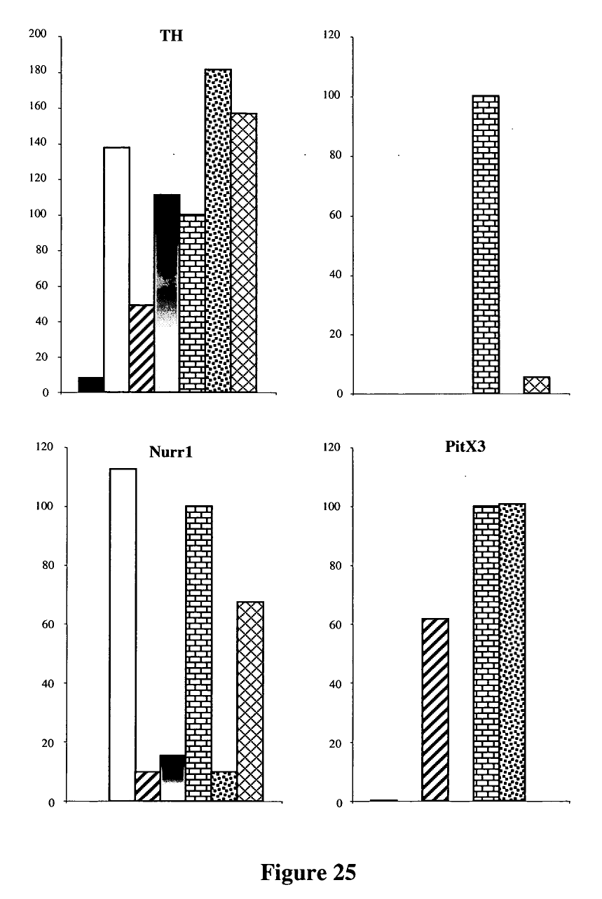

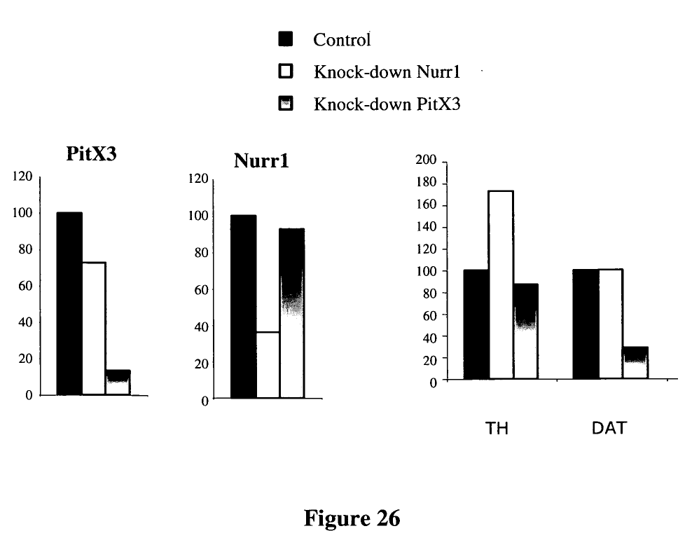

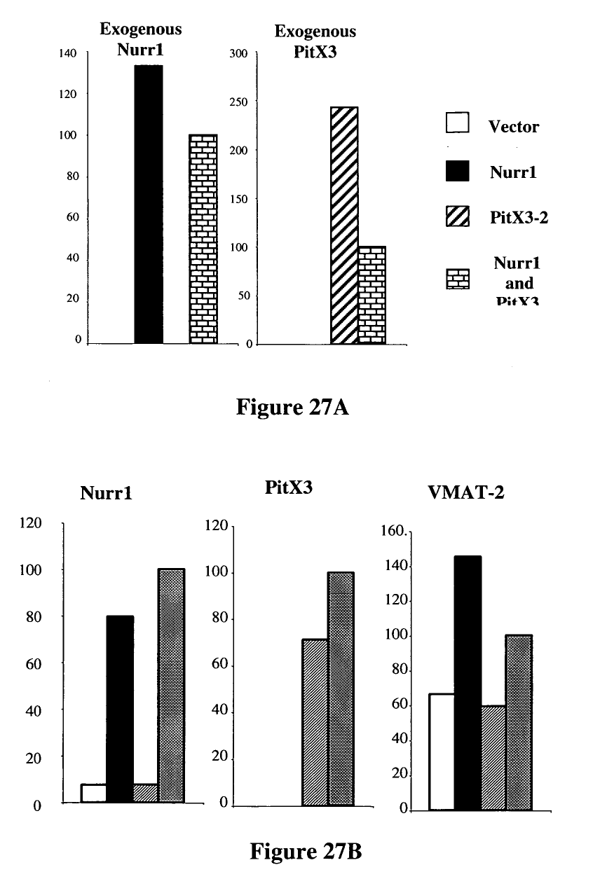

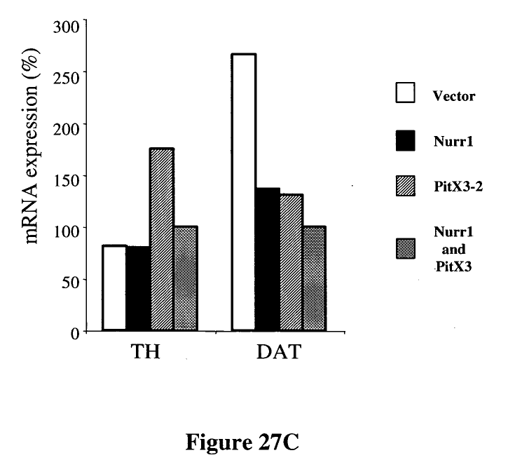

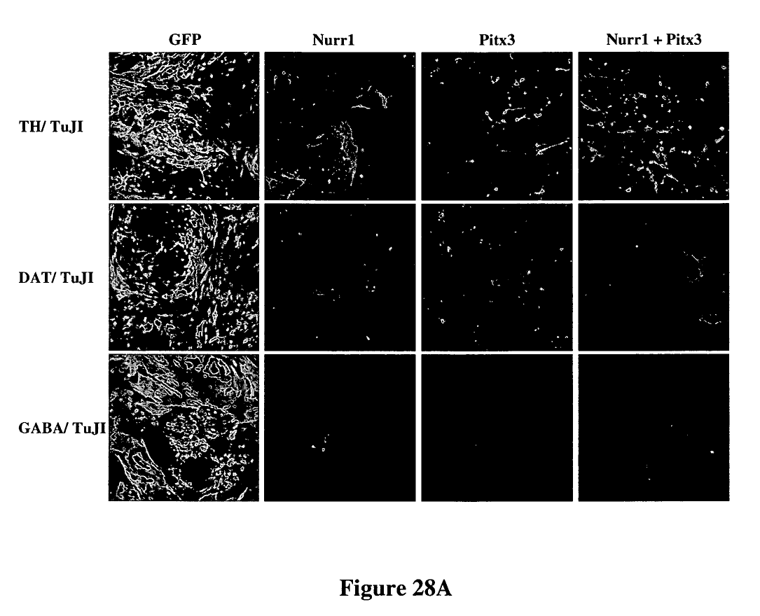

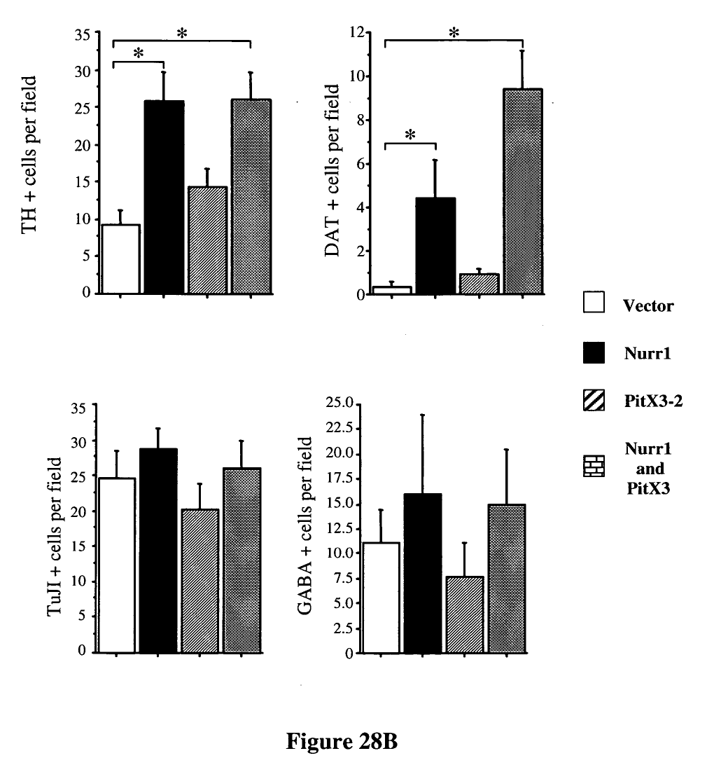

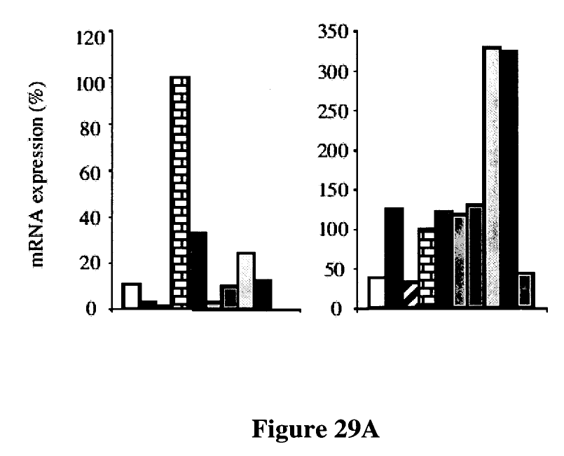

To select for ES subclones homozygous for the trapped DJ-1 allele, clone F063A04 was exposed to high-dose G418 (4 mg/ml) (Mizushima et al. 2001). Several homozygous mutant ES subclones (that have undergone gene conversion at the DJ-1 locus) were identified by Southern blotting (FIG. 12B). To confirm that the trapped allele leads to the loss of wild-type DJ-1 protein, cell lysates from ‘knockout’ homozygous clones as well as the parental heterozygous clone were analyzed by Western blotting using polyclonal antibodies to the amino terminal region of DJ-1 (amino acids 64-82) or full-length protein. Neither full-length nor truncated DJ-1 protein products were detected in ‘knockout’ clones (FIG. 12C), consistent with instability of the predicted truncated DJ-1 product, and no full-length DJ-1 RNA was detected in the mutant cultures (FIG. 17). An anti-DJ-1 rabbit polyclonal antibody was generated against the synthetic polypeptide QNLSESPMVKEILKEQESR (SEQ ID NO:5), which corresponds to amino acids 64-82 of the mouse protein. Antiserum was produced using the Polyquick antibody production service (Zymed). The antiserum was affinity purified on a peptide-coupled Sulfolink column (Pierce) per the manufacturer's instructions. Antibody was used at a dilution of 1:200 for immunohistochemistry and Western blotting was performed as described in Staropoli et al. 2003. Immunohistochemistry was performed with a rabbit polyclonal antibody to TH (PelFreez; dilution 1:1000), a mouse monoclonal antibody to TujI (Covance, dilution 1:500), and a rabbit polyclonal antibody to GABA (Sigma, dilution 1:1000). Western blotting was performed using cleaved PARP polyclonal antibody (Cell Signaling, dilution 1:500), monoclonal DJ-1 antibody (Stressgen, dilution 1:1000) and β-Actin (Sigma, 1:500). In contrast, heterozygous and wild-type (control) ES cells express high levels of DJ-1. Initial phenotypic analysis of DJ-1-deficient ES subclones indicated that DJ-1 is non-essential for the growth rate of ES cells in culture, consistent with the viability of humans with a homozygous DJ-1 mutation.

### EXAMPLE 11

### DJ-1 Protects Cells from Oxidative Stress and Proteasomal Inhibition

To investigate the role of DJ-1 in the oxidative stress response in vivo, DJ-1-deficient homozygous mutant (‘knockout’) cells and DJ-1 heterozygote (‘control’) ES cell clones were analyzed for cell viability in the context of increasing concentrations of H₂O₂. ES cells plated in 96-well format (2.3×104 cells/well) were treated for 15 hours with H₂O₂ in ES media deficient in b-mercaptoethanol (Abeliovich et al. 2000). Cell viability (as a percent of untreated control) was determined by MTT assay in triplicate (Fezoui et al. 2000). Annexin V/Propidium Iodide (Molecular probes) staining was performed per the manufacturer's instructions. For dihydrorhodamine-123 staining (DHR, Molecular probes) (Walrand et al. 2003), cells were preincubated for 30 min with DHR (5 mM), washed with PBS, then treated with H₂O₂ in ES media deficient in b-mercaptoethanol for 15 min at 37° C. The FACS analysis was performed using a FACSTAR sorter (Becton Dickinson). Dopamine uptake assays were performed essentially as described in Farrer et al. 1998. Reported values represent specific uptake from which non-specific uptake, determined in the presence of mazindol, was subtracted, and normalized for protein content (BCA kit, Pierce).

Primary midbrain embryonic cultures were prepared and transduced with lentiviral vectors as described (Staropoli et al. 2003). DJ-1 shRNA vector was generated by insertion of annealed oligonucleotides 5′-TGTCACTGTTGCAGGCTTGGTTCAAGAGACCAAGCCTGCAACAGTG ACTTTTTTC-3′ (SEQ ID NO:6) and 5′-ACAGTGACAACGTCCGAACCAAGTTCTCTGGTTCGGACGTTGTCACTG AAAAAAGAGCT-3′ (SEQ ID NO:7) into the LentiLox vector (Rubinson et al. 2003). For cellular dopamine quantification, cultures were incubated in standard differentiation media supplemented with L-DOPA (50 mM) for 1 hour to amplify dopamine production as described (Pothos et al. 1996). Subsequently cells were washed in phosphate buffered saline and then lysed in 0.2 M perchloric acid. Dopamine levels were quantified by HPLC (Yang et al. 1998) and normalized for protein content as above.

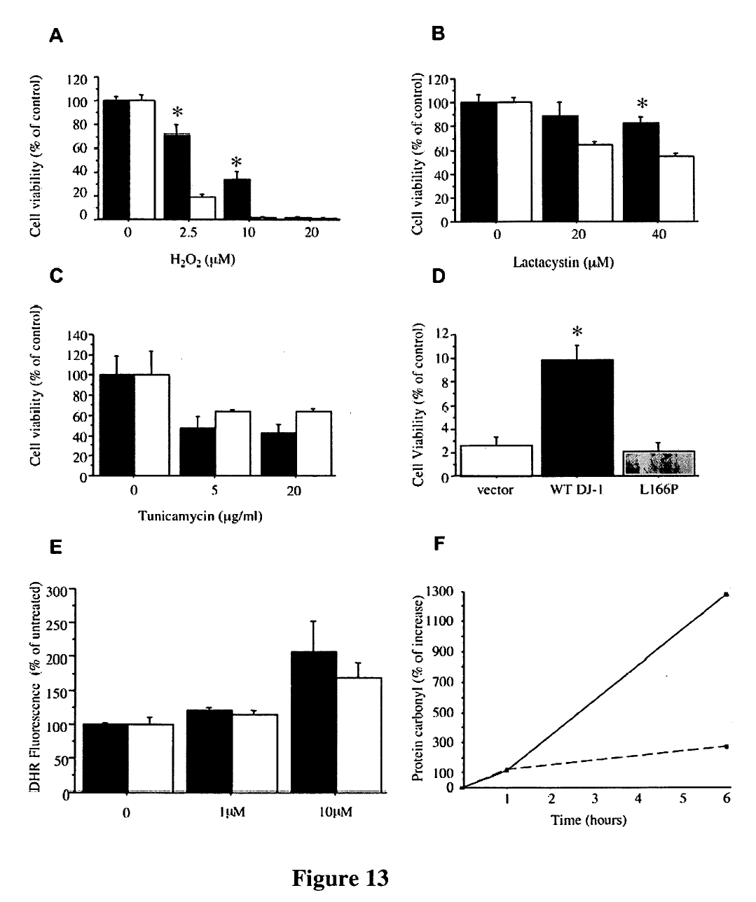

Heterozygous cells were used as controls because the ‘knockout’ subclones were derived from these. Cell viability was initially determined by MTT assay in triplicate (Fezoui et al. 2000). Exposure to H₂O₂ led to significantly greater toxicity in DJ-1 deficient cells; similar results were obtained with multiple DJ-1 deficient subclones in independent experiments (FIGS. 12D and 13A). Untreated heterozygous and homozygous cells displayed comparable viability in the MTT assay in the absence of toxin (FIG. 17). Consistent with the MTT assay, fluorescence activated cell sorting (FACS) analysis of cells stained with Annexin V (AV) and propidium iodide (PI) revealed increased cell death of knockout cells (relative to heterozygous cells) in the context of H₂O₂ exposure (FIG. 12E). The increase in AV-positive cells implicated an apoptotic mechanism of cell death (FIG. 12F). Furthermore, in the context of H₂O₂, knockout cells displayed potentiated cleavage of Poly(ADP-ribose)polymerase-1 (PARP) in a pattern indicative of an apoptotic death program (Gobeil et al. 2001) (FIG. 12G).

Additional toxin exposure studies demonstrated that DJ-1 deficient cells were sensitized to the proteasomal inhibitor lactacystin (FIG. 13B), as well as copper (FIG. 17), which catalyzes the production of ROS. We did not observe altered sensitivity to several other toxins including tunicamycin (an inducer of the unfolded protein response in the endoplasmic reticulum; FIG. 13C), staurosporine (a general kinase inhibitor that induces apoptosis), or cycloheximide (an inhibitor of protein translation).

### EXAMPLE 12

### Wild-Type But Not PD-Associated L166P Mutant DJ-1 Protects Cells from Oxidative Stress

To confirm that altered sensitivity to oxidative stress is a consequence of the loss of DJ-1, we performed ‘rescue’ experiments by overexpressing wild-type or mutant human DJ-1 in ‘knockout’ ES cells. Plasmids encoding human wild-type DJ-1, PD-associated L166P mutant DJ-1, or vector alone, were transiently transfected into DJ-1 deficient clones, and these were subsequently assayed for sensitivity to H₂O₂ using the MTT viability assay. DJ-1 deficient cells transfected with a vector encoding wild-type human DJ-1 were effectively ‘rescued’ in terms of viability in the presence of H₂O₂ (FIG. 13D); Thus, viability in ‘rescued’ knockout cells mimicked the viability of control (heterozygous) cells in the context of H₂O₂ treatment (FIGS. 13A, D). In contrast, transfection of a vector encoding the PD-associated L166P mutant DJ-1 did not significantly alter the viability of H₂O₂-treated knockout cells. Baseline cell viability in the absence of toxin exposure was not altered by DJ-1 overexpression, and Western blotting of lysates from transfected cells with an antibody specific to human DJ-1 demonstrated that transfected human wild-type and L166P mutant DJ-1 accumulated comparably.

### EXAMPLE 13

### DJ-1 Deficiency Does Not Alter the H₂O₂-Induced Intracellular ROS Burst

We quantified the accumulation of ROS in response to H₂O₂ treatment in mutant and heterozygous control cells using the ROS-sensitive fluorescent indicator dye dihydrorhodamine-123 (DHR) and FACS analysis. Initial ROS accumulation (at 15 minutes after stimulation) appeared unaltered in DJ-1 deficient cells in comparison to control heterozygous cells (FIG. 13E). Consistent with this, accumulation of protein carbonyls, an index of oxidative damage to proteins (Sherer et al. 2002), appeared normal initially (at 1 hour after toxin exposure; FIG. 13F). However at 6 hours after toxin exposure, a point at which knockout cells already display increased apoptosis (as determined by PARP cleavage; FIG. 12G), protein carbonyl accumulation robustly increased in the DJ-1 deficient cells. These data suggest that initial ROS accumulation was not altered by DJ-1 deficiency, but that the mutant cells were unable to appropriately cope with the consequent damage. Consistent with this we failed to detect antioxidant or peroxiredoxin activity with purified DJ-1 protein in vitro (Shendelman et al.).

### EXAMPLE 14

### DJ-1 is Required for Survival of ES-Derived Dopamine Neurons

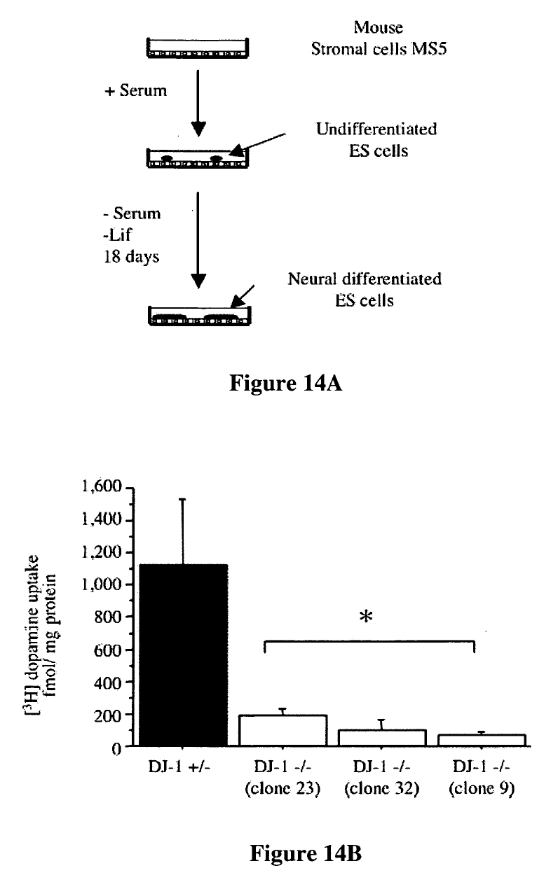

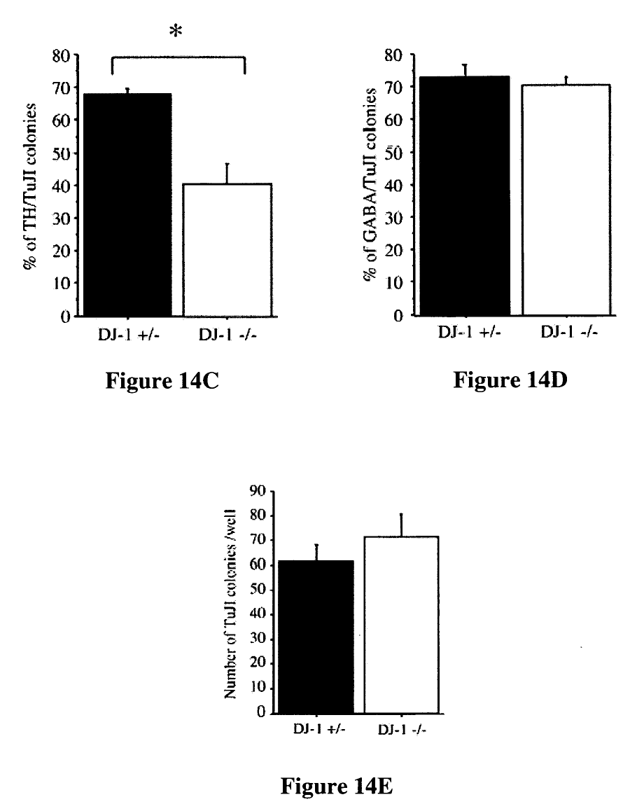

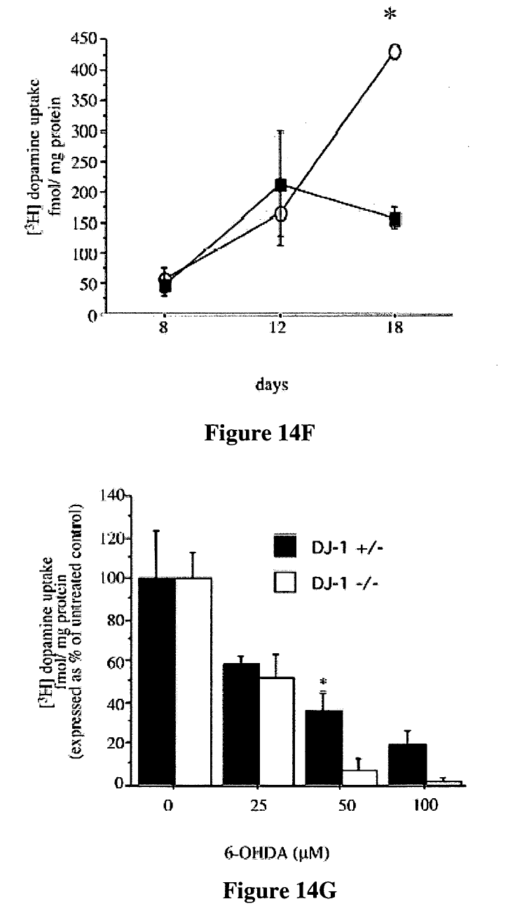

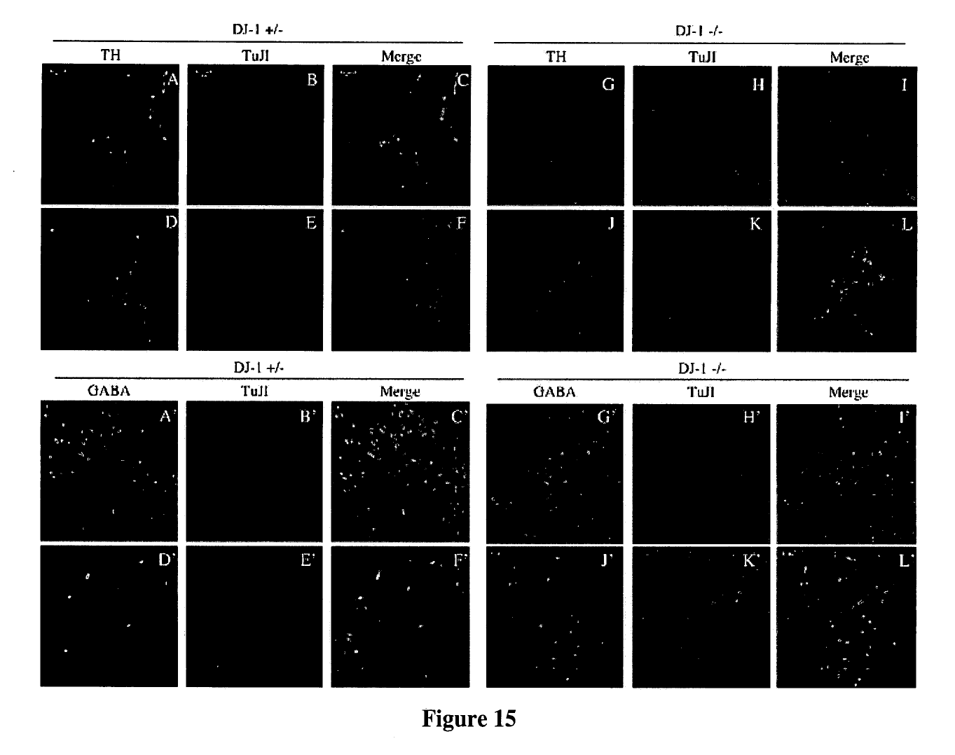

Several methods have been established for the differentiation of ES cells into dopamine neurons (DN) in vitro (Morizane et al. 2002). To extend our analysis of DJ-1 function to DNs, we differentiated DJ-1-deficient ES cells or control heterozygous cells into DNs in vitro by co-culture with stromal cell-derived inducing activity (SDIA; FIG. 14A) (Morizane et al. 2002; Barberi et al. 2003). Dopamine neurons were quantified by immunohistochemistry for tyrosine hydroxylase (TH; a marker for dopamine neurons and other catecholaminergic cells), or by analysis of dopamine transporter uptake activity (a quantitative dopamine neuron marker) (Han et al. 2003). Production of dopamine neurons appeared to be significantly reduced in DJ-1-deficient ES cell cultures relative to parental heterozygous cultures at 18 days in vitro as determined both by dopamine uptake and TH immunoreactivity (FIGS. 14B and 14C, and 15A-L). In contrast, general neuronal production did not appear altered in this assay in terms of the post-mitotic neuronal marker Tuj1 (FIGS. 14E and 15A-L′), and other neuronal subtypes appeared normal, including GABAergic (FIGS. 14D and 15A′-L′) and motor neurons (HB9-positive). To investigate whether the reduction in dopamine neurons in DJ-1 deficient cultures is due to defective generation or survival, a time course analysis was performed. We found that at early time points (8 and 12 DIV) dopamine uptake activity was comparable in wild-type and DJ-1 deficient cultures, whereas subsequently the DJ-1 deficient cultures appeared defective (FIG. 14F). Consistent with this, intracellular dopamine accumulation (as quantified using high performance liquid chromatography; HPLC) was significantly reduced in DJ-1 deficient cultures (6.4±1.5 ng dopamine/mg protein) relative to control heterozygous cultures (66.0±17.4 ng/mg) at 35 DIV. These data strongly suggest that DJ-1 deficiency leads to loss of dopamine neurons, rather than simply to downregulation of cell marker expression.

Dopamine neuron cultures from DJ-1-deficient or heterozygous control ES cultures at 9 DIV were exposed to oxidative stress in the form of 6-hydroxydopamine (6-OHDA), a dopamine neuron-specific toxin that enters dopamine neurons through the dopamine transporter and leads to oxidative stress and apoptotic death (Dauer and Przedborski 2003). DJ-1 deficient dopamine neurons displayed an increased sensitivity to oxidative stress in this assay (FIG. 14G). Post-hoc analysis of the data indicated that the difference among genotypes is maximal at an intermediate dose of toxin (50 μM); at the highest dose of 6-OHDA employed (100 μM) the difference is lessened, indicating that DJ-1-mediated protection is limited. Although we cannot exclude a role for DJ-1 in the late stage differentiation of dopamine neurons, these data suggest that DJ-1 deficiency leads to reduced dopamine neuron survival and predisposes these cells to endogenous and exogenous toxic insults.

### EXAMPLE 15

### RNAi ‘Knockdown’ of DJ-1 in Midbrain Embryonic Dopamine Neurons Leads to Increased Sensitivity to Oxidative Stress

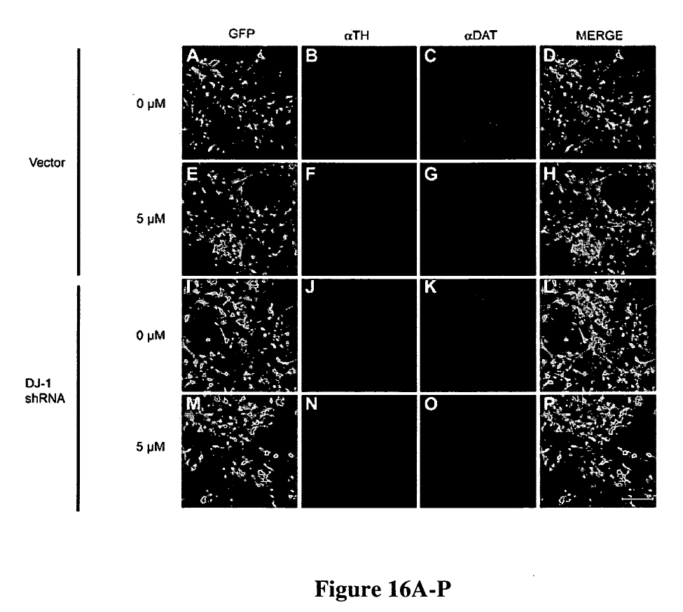

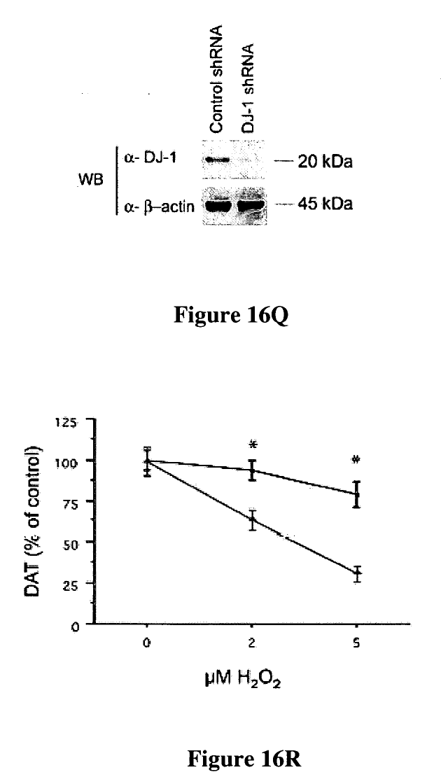

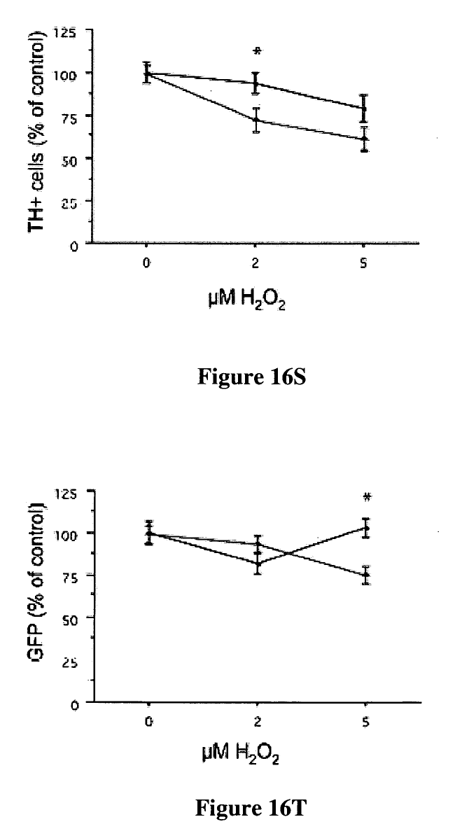

To confirm the role of DJ-1 in primary midbrain dopamine neurons, DJ-1 expression was inhibited by RNA interference (RNAi) in embryonic day 13 (E13) murine primary midbrain cultures by lentiviral transduction of short hairpin RNAs (shRNA) (Rubinson et al. 2003). E13 midbrain cultures (Staropoli et al. 2003) were transduced with a lentiviral vector that encodes a fluorescent marker gene, eGFP, along with short hairpin RNAs (shRNA) homologous to murine DJ-1. DJ1 -shRNA virus-infected cells displayed efficient silencing of DJ-1 gene expression to 10-20% of control vector-infected cultures (as determined by Western blotting; FIG. 16Q). Transduction efficiency, as assessed by visualization of the fluorescent eGFP marker, exceeded 95% in all cases (FIG. 16I). After 7 days in vitro (DIV7), cultures were exposed to hydrogen peroxide for 24 hours and then evaluated for dopamine neuron survival as quantified by immunostaining for TH and DAT.

Midbrain cultures transduced with DJ-1 shRNA virus and control vector transduced cells displayed similar numbers of TH-positive neurons in the absence of exposure to H₂O₂ (FIG. 16A-D, I-L, R-S). In contrast, in the presence of H₂O₂, DJ-1-deficient cultures displayed significantly reduced dopamine neuron survival as quantified by immunohistochemistry for TH or DAT (FIG. 16E-H, M-P, R-S). Similar results were obtained in three independent studies. The reduction in DAT immunoreactivity appears to be more robust than the reduction in TH cell number in the context of hydrogen peroxide; this may reflect the differential localization of DAT to dopamine neuron processes, whereas TH is primarily in the cell body.

Non-dopaminergic cells in the E13 primary midbrain cultures are predominantly GABAergic neurons (90-95%) (Staropoli et al. 2003). Total embryonic midbrain neurons transduced with either DJ-1 shRNA or vector displayed comparable survival in the context of toxin exposure, suggesting that DJ-1 deficiency leads to a relatively specific alteration in dopamine neuron survival (FIG. 16T). These data are consistent with the analyses of ES-derived dopamine neurons above and indicate that DJ-1 is required for the normal survival of midbrain dopamine neurons in the context of toxin exposure.

### EXAMPLE 16

### Nurr1 and PitX3 Regulate the Maturation of ES-Derived Dopamine Neurons

Midbrain dopamine neurons (mDNs) play a central role in the regulation of voluntary movement and their degeneration is associated with Parkinson's disease (PD). Cell replacement therapies, and in particular embryonic stem (ES) cell-derived replacement mDN neurons, offer a potential therapeutic venue for PD. We sought to identify genes that can induce maturation of ES cultures to mDNs. Expression analyses and loss-of-function studies in mice have implicated several transcription factors in the specification and maturation of mDNs, including Nurr1 (Wallen et al., 2003), PitX3 (Hwang et al., 2003; Nunes et al., 2003; van den Munckhof et al., 2003; Smidt et al., 2004), Lmx1b (Smidt et al., 2000), and Engrailed (En) −1 and −2 (Simon et al., 2001; Alberi et al., 2004). However, none of these factors appears sufficient alone to induce the mature mDN phenotype, suggesting a more complex regulatory network. Here we show that Nurr1 and PitX3 cooperatively promote terminal maturation of mDNs in murine and human embryonic stem (ES) cell cultures. We present evidence that cooperative DNA binding of these factors to transcription promoter elements of late mDN genes underlies their synergistic activity in mDN maturation.

A number of regulatory factors have been implicated in the maturation of mDNs. Expression of the orphan nuclear receptor/transcription factor Nurr1 is initiated in postmitotic mDN precursors at embryonic day 10.5 (E10.5) in the mouse, just preceding expression of tyrosine hydroxylase (TH), the rate-limiting enzyme in dopamine synthesis (Wallen et al., 2003); at subsequent stages of development and through adulthood Nurr1 is expressed more broadly in the adult brain. Nurr1-deficient animals fail to express TH in mDN but other mDN markers such as PitX3, Lmx1b, Engrailed-1, and Aldh2, remain unaltered (Zetterstrom et al., 1997; Saucedo-Cardenas et al., 1998; Castillo et al., 1998), and thus Nurr1 is not required for the mDN fate. Nurr1-deficient mice also fail to express markers for subsequent mDN maturation, such as the dopamine transporter (DAT), and mDNs are lost by birth.

Expression analyses and loss-of-function knockout mouse studies have implicated additional transcription factors in the specification and maturation of mDNs, including the homeodomain proteins Lmx1b (Smidt et al., 2000), PitX3 (Hwang et al., 2003; Nunes et al., 2003; van den Munckhof et al., 2003; Smidt et al., 2004), En-1 and En-2 Alberi et al., 2004). None of these factors are uniquely expressed in mDNs, nor are they required for relatively early developmental events such as mDN cell fate determination; however, all appear necessary for the maturation and survival of the cells.

Overexpression of Nurr1 alone in neuronal cell lines (Wagner et al., 1999), primary neuronal precursor cells (Sakurada et al., 1999), or ES cultures (Kim et al., 2002; Sonntag et al., 2004) appears to promote the expression of a subset of mDN markers in vitro, including TH, but this may reflect a broad proneural activity (Kim et al., 2003). PitX3 overexpression in undifferentiated ES cultures or in neuron progenitor cells is also not sufficient to induce a mature mDN phenotype but may promote a small subset of markers (Sakurada et al., 1999; Chung et al., 2005). Thus, we hypothesized that multiple transcription factors may collaborate within a network to induce late events in mDN maturation.

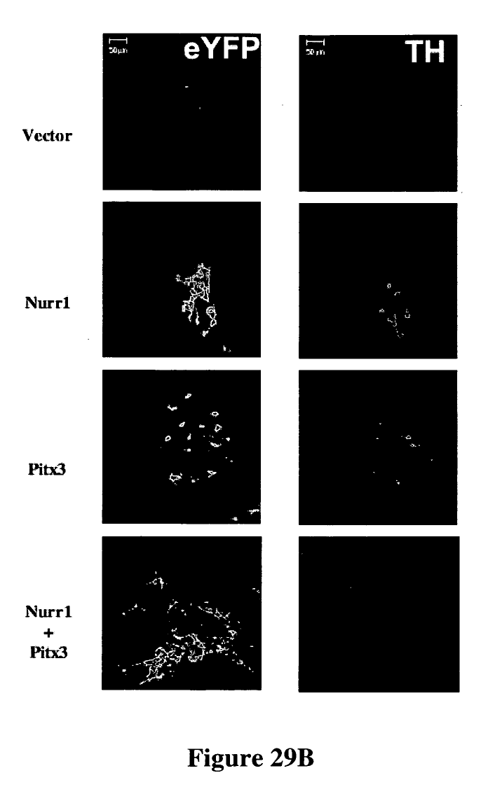

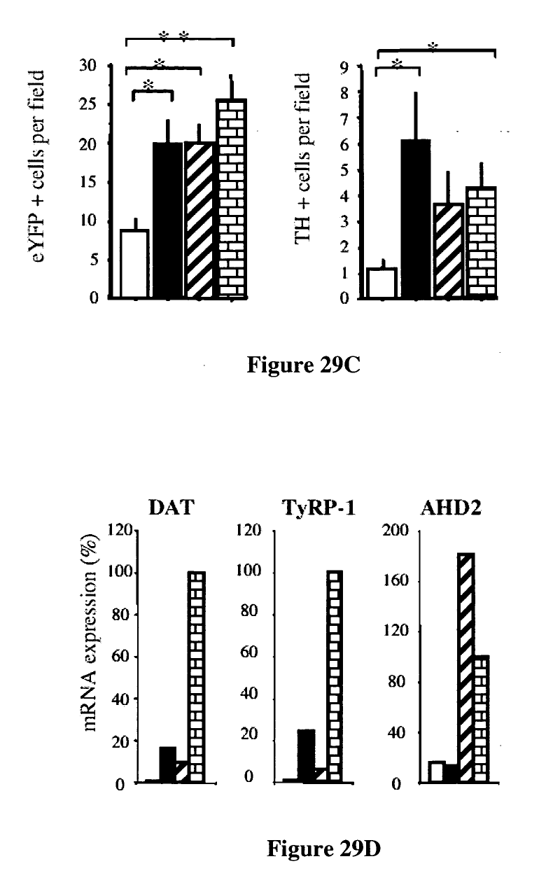

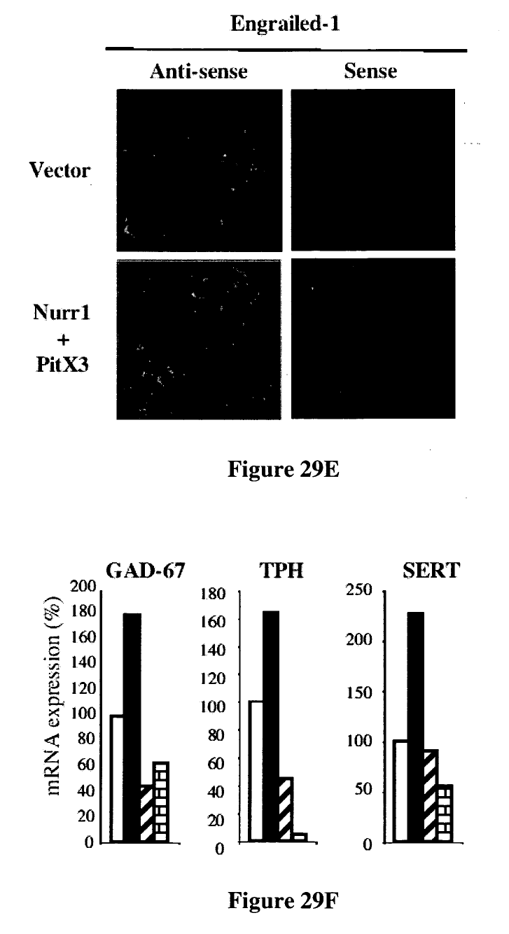

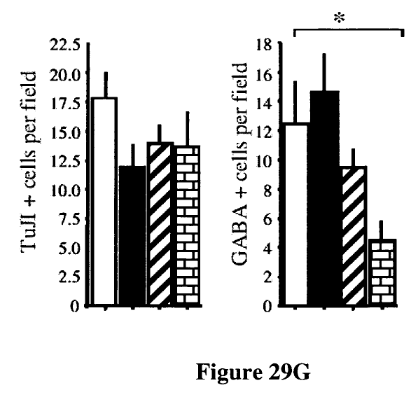

We sought to test this hypothesis using the ES culture differentiation as a model system. Murine ES cultures (MM13) differentiated in vitro by an embryoid body (EB)-based protocol recapitulate the complete temporal expression pattern of mDN developmental markers, albeit inefficiently (Lee et al., 2000). Lentiviral expression vectors (Zennou et al., 2001) that harbor Nurr1, PitX3, Lmx1b, En-1 or control vector were introduced at the neural precursor stage, approximating the temporal expression pattern of the endogenous genes and leading to transgene overexpression in over 95% of cells. No single factor appeared effective in inducing the expression of the late marker, DAT, as quantified by real-time reverse transcription-polymerase chain reaction (rtPCR; FIG. 29a). Surprisingly, the combined overexpression of Nurr1+PitX3 was uniquely effective in the induction of late marker expression as determined by rtPCR (FIG. 29a) or immunohistochemistry and FACS analysis.

Similar results were obtained with a second independent murine ES cell line, DY-1, that expresses a dopamine-neuron specific fluorescent marker, eYFP, under the regulation of a Cre recombinase gene ‘knocked-in’ to the DAT locus 20. Overexpression of Nurr1 and PitX3 together in DY-1 cultures led to significantly more eYFP+cells, as determined by immunohistochemistry (FIG. 29b-c) or FACS analysis. In contrast, Nurr1 alone increased the expression of TH, consistent with prior reports of a direct role for Nurr1 in TH induction (Sakurada et al., 1999; Kim et al., 2002; Chung et al., 2002).

Further analyses of additional markers for mDN as well as other cell phenotypes in the EB-differentiated cultures indicated that Nurr1 and PitX3 specifically instruct mDN maturation. Thus, co-overexpression of Nurr1 and PitX3 induced late mDN maturation markers such as DAT and tyrosinase related protein-1 (TyRP-1; FIG. 29d) (Miranda et al., 1984). In contrast, earlier phenotypic markers of mDN such as TH and aldehyde dehydrogenase-2 (AHD-2) appear induced by Nurr1 alone or PitX3 alone, respectively (FIG. 29a-d). Global gene expression profiling of EB-differentiated cultures transduced with Nurr1 and PitX3 using oligonucleotide microarrays indicated that the maturation markers DAT and TyRP-1 are among the most highly induced genes in cells transduced with both Nurr1 and PitX3 (within the top 0.2% of annotated genes induced by the combined expression of Nurr1 and PitX3; Table 1).

TABLE 1
Primers used for real-time PCR

| | |
|---|---|
| Description | Sequence |

| | |
|---|---|
| Mus musculus MRET forward primer | 5′ - CATCAGCGGAAATGACCTTCTG - 3′ |
| Mus musculus MRET reverse primer | 5′ - CTTGAAAGAGTCCACAGGAACC - 3′ |
| Lentiviral 3′ forward primer | 5′ - GCAATACAGCAGCTACCAATGCTG - 3′ |
| Lentiviral 3′ forward primer | 5′ - GGCTAAGATCTACAGCTGCCTTGT - 3′ |
| Exogenous nurr Forward primer | 5′ - CATGGACTACAAGGACGACGATGAC - 3′ |
| Exogenous nurr reverse primer | 5′ - AATCGGAGCTGTATTCTCCCGAAG - 3′ |
| Mus musculus tyrosine hydroxylase forward primer | 5′ - TTGAAAAACTCTCCACGGTGTACT - 3′ |
| Mus musculus tyrosine hydroxylase reverse primer | 5′ - TACGGGTCAAACTTCACAGAGAAT - 3′ |
| Mus musculus DAT (Slc6a3) forward primer | 5′ - GTATGCTCTGATGCCATCCAT - 3′ |
| Mus musculus DAT (Slc6a3) reverse primer | 5′ - TGATGCACATAGCAGCAACTC - 3′ |
| Mus musculus Nurr 1 (Nr4a1) forward primer | 5′ - CTCCCTCCATGAGGGTCTG - 3′ |
| Mus musculus Nurr 1 (Nr4a1) reverse primer | 5′ - TCTTCGGCTTCGAGGGTAAA - 3′ |
| Mus musculus Ptx3 forward primer | 5′ - TTTCGCAACGGGTTTGCCGC - 3′ |
| Mus musculus Ptx3 reverse primer | 5′ - AAGGTCGCCTCTAGCTCCTGTAG - 3′ |
| Mus musculus vmat2 (Slc18a2) forward primer | 5′ - AATATCTACGGACTCATCGCTCCC - 3′ |
| Mus musculus vmat2 (Slc18a2) reverse primer | 5′ - GCATACACACTCCCATAGACAGAC - 3′ |
| Mus musculus Gad67 forward primer | 5′ - GCCGGGGCTGCGCTTGGCTTTG - 3′ |
| Mus musculus Gad67 reverse primer | 5′ - TGGCGGTGCTTCCGGGACATGAGC - 3′ |
| Mus musculus nestin forward primer | 5′ - GAAACTCTCCCTGACTCTACTCCC - 3′ |
| Mus musculus nestin reverse primer | 5′ - CTT GCT GCT CTT CCT CTG GT - 3′ |
| Mus musculus DAT (Slc6a3) forward primer | 5′ - AAC TCC ACC CTC ATC AAC CCA - 3′ |
| Mus musculus DAT (Slc6a3) reverse primer | 5′ - AAG CCC ACA CCT TTC AGG ACA - 3′ |
| Mus musculus Gfra1 forward primer | 5′ - TCC AAA GGG AAC AAC TGC CTC GAT - 3′ |
| Mus musculus Gfra1 reverse primer | 5′ - AGC TGT GCT TGG CTG GAA CTT T - 3′ |
| Mus musculus Gdnf forward primer | 5′ - ACG CTT GGT GGT TGA TTC TGG A - 3′ |
| Mus musculus Gdnf reverse primer | 5′ - AAC TTG CTT CCT GTT TCT GAG GGC - 3′ |
| Mus musculus Bdnf forward primer | 5′ - ACG ACA TCA CTG GCT GAC ACT T - 3′ |
| Mus musculus Bdnf reverse primer | 5′ - TCC AAA GGC ACT TGA CTG CTG A - 3′ |
| Mus musculus beta-actin forward primer | 5′ - TGA GAG GGA AAT CGT GCG TGA CAT - 3′ |
| Mus musculus beta-actin reverse primer | 5′ - ACC GCT CGT TGC CAA TAG TGA TGA - 3′ |
| Egfp forward primer | 5′ - AAGCTGACCCTGAAGTTCATCTGC - 3′ |
| Egfp reverse primer | 5′ - CACCTTGATGCCGTTCTTCTGCTT - 3′ |
| Exogenous Pitx3 forward primer | 5′ - TTTCGCAACGGGTTTGCCGC - 3′ |
| Exogenous Pitx3 reverse primer | 5′ - AAGGTCGCCTCTAGCTCCTGTAG - 3′ |
| Mus musculus aadc forward primer | 5′ - TGG CAC AGT CCC TAT TTC TTC GCT - 3′ |
| Mus musculus aadc reverse primer | 5′ - AAT GCA GCC AAT AGC ACC ACA CAG - 3′ |
| Mus musculus tryptophan Hydroxylase forward primer | 5′ - GGC GTA TGG AGC AGG GTT ACT TTC - 3′ |
| Mus musculus tryptophan Hydroxylase reverse primer | 5′ - AAT GCT CTG CGT GTA GCG GTT G - 3′ |
| Mus musculus Chat forward primer | 5′ - AGC GAG CCT TGT TGA CAT GAG AGA - 3′ |
| Mus musculus Chat reverse primer | 5′ - TCA AGA TTG CTT GGC TTG GTT GGG - 3′ |
| Mus musculus Gfap forward primer | 5′ - TCA ACG TTA AGC TAG CCC TGG ACA - 3′ |
| Mus musculus Gfap reverse primer | 5′ - TCT GTA CAG GAA TGG TGA TGC GGT - 3′ |

Midbrain DNs can be differentiated from other DN populations by the expression of a number of midbrain markers such as En-1 and Lmx1b. Consistent with a midbrain phenotype, these markers are expressed in over 95% of cells in the EB-differentiated cultures (FIG. 29e). Markers for other neuronal cell types, such as GABAergic neurons (glutamic acid decarboxylase-67; GAD-67) and serotonergic neurons (tryptophan hydroxylase; TPH), are not induced by Nurr1+PitX3 (FIG. 29f-g). Furthermore, GABAergic neuron number was significantly reduced in the PitX3 and Nurr1 doubletransduced cultures. Nurr1 and PitX3 overexpression did not alter the overall number of neurons, as determined by TuJI staining (FIG. 29g). Neuron survival, as quantified by staining with the nuclear marker Topro-3, similarly appeared unaltered in the transduced cells. There does not appear to be cross-regulation between Nurr1 and PitX3: Nurr1 overexpression failed to induce endogenous PitX3 and vice versa (Smidt et al., 2000; Kim et al., 2002).

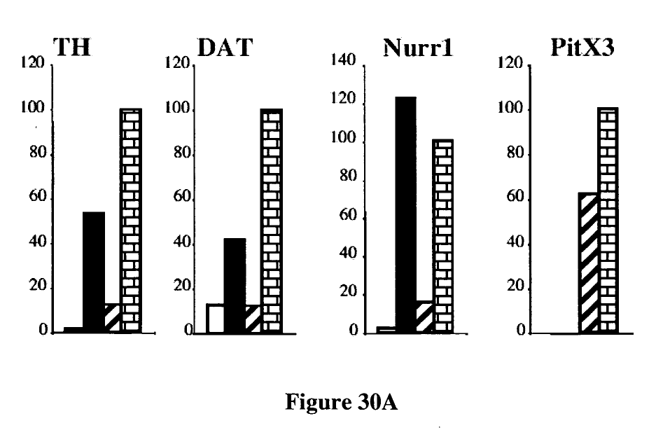

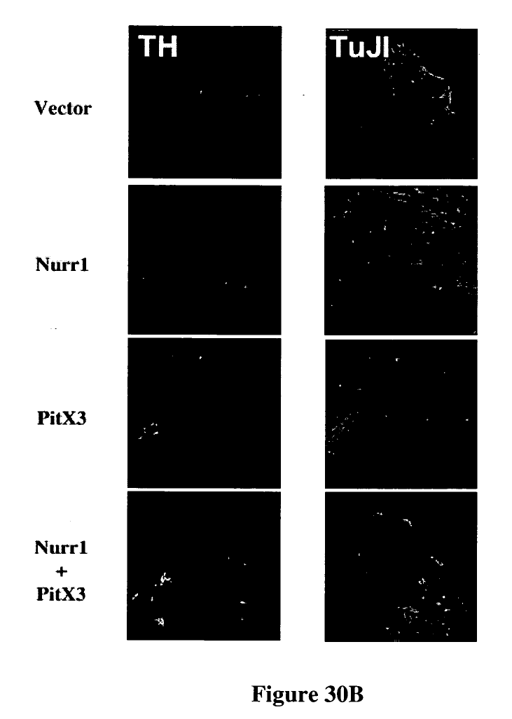

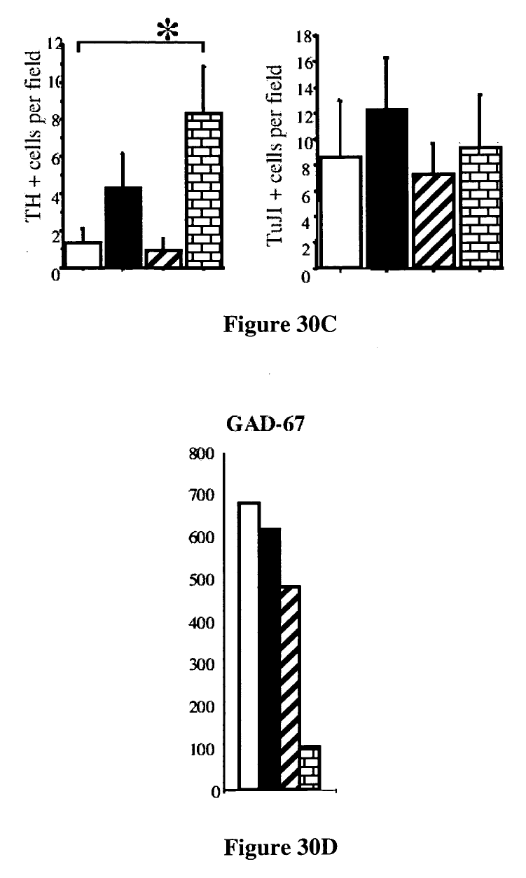

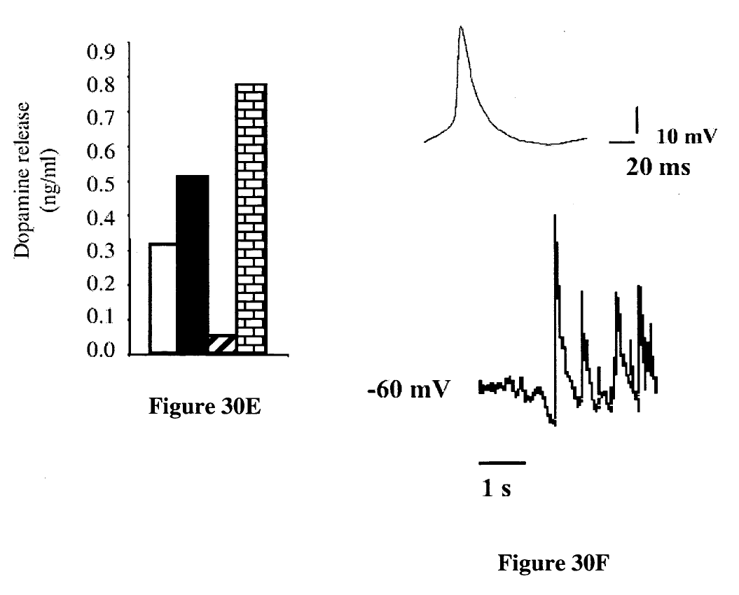

To determine whether the synergistic action of Nurr1 and PitX3 represents a general mechanism for the induction of mDN maturation, we extended our analyses to human ES cell cultures. Prior studies have described the differentiation of human ES cultures into TH+ cells in vitro (Perrier et al., 2004; Yan et al., 2005), but the roles of transcription regulatory factors have not been explored. To functionally dissect the role of transcription factor regulators implicated in the maturation of human ES cultures to mDNs, we used a coculture-based differentiation assay, as described for both rodent25 and primate ES cultures (Perrier et al., 2004; Takagi et al., 2005), in which ES cells are cultured with bone-marrow derived stromal cells (stromal derived induction activity, SDIA). At the neural precursor stage of the SDIA protocol, cells were transduced with lentiviral vectors that harbor Nurr1, PitX3, both, or control vector alone (green fluorescent protein; GFP), cultured for 2 additional weeks, and then analyzed for the expression of mDN maturation markers by rtPCR or immunohistochemistry. The combination of Nurr1 and PitX3 effectively promoted the maturation of mDNs derived from human ES cells as quantified by the expression of DAT as well as several other markers, including TH, the vesicular monoamine transporter-2 (VMAT-2), and amino acid decarboxylase (AADC; FIG. 30a-c). In a separate series of studies, Nurr1+PitX3 transduction promoted the maturation of human ES cultures in the context of a second differentiation method, the EB protocol.

Nurr1 and PitX3 co-transduction reduced the expression of nondopaminergic neuronal fate markers in the human ES cultures, such as glutamic acid decarboxylase (GAD-67), a marker for GABAergic neurons, and overall neuron number appeared unaltered as quantified by TujI immunostaining (FIG. 30c-d). Consistent with these data, differentiated H9 human ES cultures transduced with Nurr1 and PitX3 display increased evoked dopamine release in the context of elevated extracellular potassium (FIG. 30e). Electrophysiological analysis of Nurr1 and PitX3-transduced mDN in human ES-derived cultures using patch electrodes and current clamp techniques indicated that they display basic neuronal characteristics including depolarization-induced action potentials and burst firing patterns (FIG. 30f). These results demonstrate the feasibility of genetic modification of human ES cultures to promote maturation to the mDN phenotype.

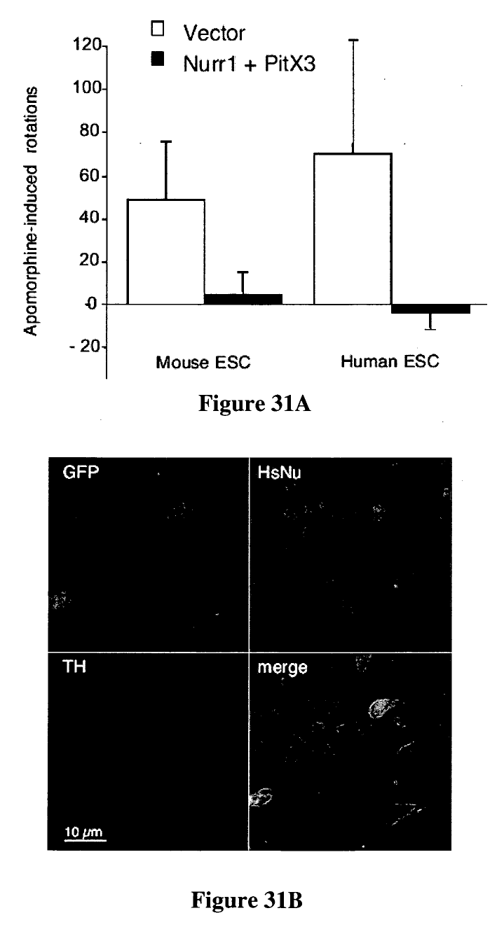

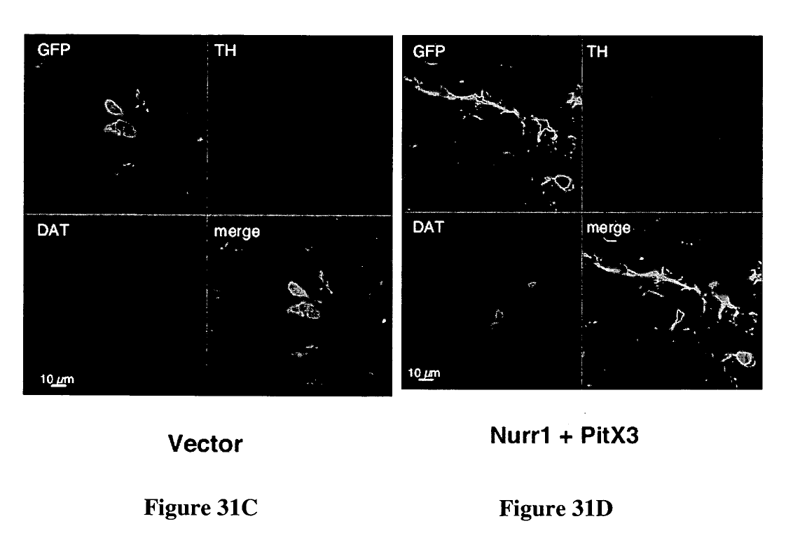

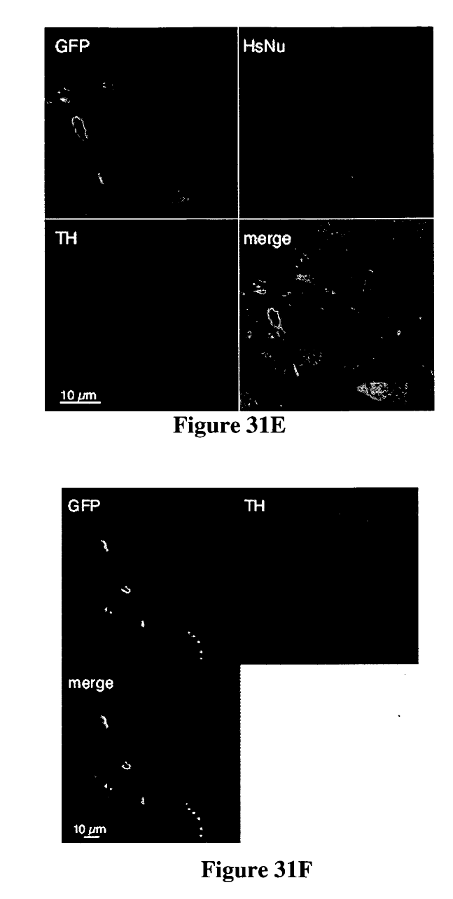

To investigate the function of Nurr1 and PitX3-transduced ES-derived cultures in vivo, either mouse or human ES cultures were differentiated to the neural precursor stage and transplanted into adult mice that had been lesioned with a unilateral intrastriatal injection of the dopamine neuron toxin 6-hydroxydopamine (6-OHDA). In the absence of transplanted cells, lesioned animals display a characteristic contralateral turning behavior (away from the side of the lesion) when exposed to the dopamine receptor agonist apomorphine (as a result of dennervation-induced hypersensitivity on the lesioned side; Nishimura et al., 2003). Transplantation of cells transduced with PitX3 and Nurr1 led to a significantly greater reduction in this turning behavior than did control vector-transduced cells at 6 weeks after cell grafting (FIG. 31a). Mouse and human ES-derived cells did not differ significantly in this functional assay. Immunohistochemical analysis confirmed the presence of mature engrafted cells at the site of transplantation (FIG. 31b-e). TH immunoreactivity was increased at the site of the graft in the context of the Nurr1 and PitX3 transduced cells relative to vector alone at 6 weeks post-implantation in both human (FIG. 31b-e) and mouse transplants. Furthermore, transplanted TH+ processes could be visualized both within the graft and extending into the surrounding striatal tissue (FIG. 31e-f).

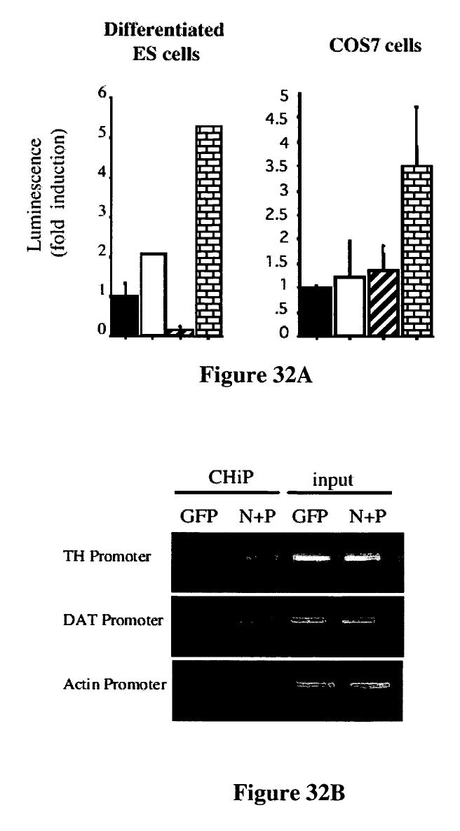

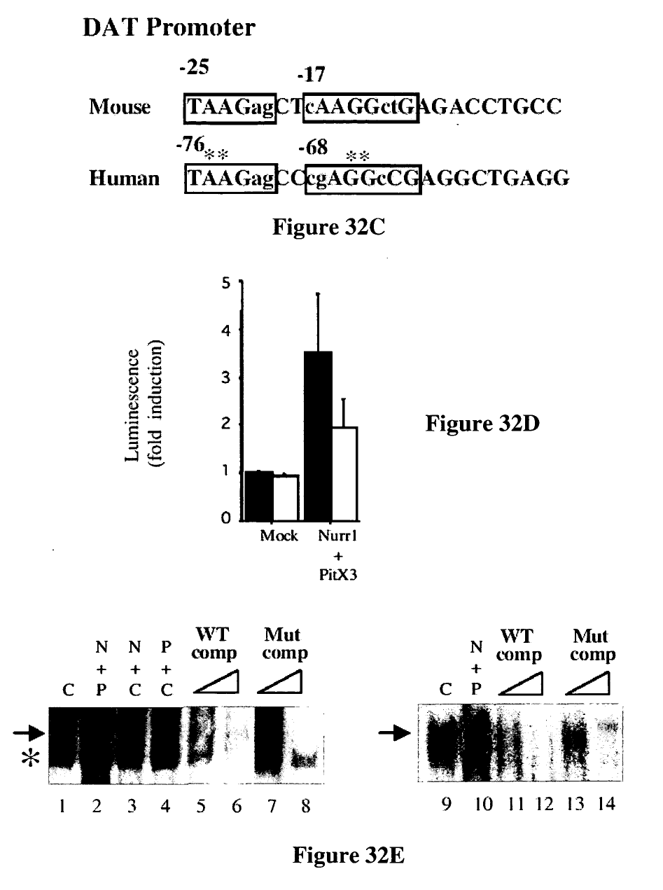

We hypothesized that the cooperative activity of Nurr1 and PitX3 in midbrain dopamine neuron maturation may be a consequence of cooperative transcriptional activation at mDN-specific genes. Indeed, Nurr1 has previously been implicated in the direct activation of DAT transcription (Sacchetti et al., 2001). To quantify the activities of Nurr1 and PitX3 on DAT gene regulatory sequences, a luciferase assay plasmid that harbors an 8.3 kb region upstream of the human DAT gene25 was transfected into cells overexpressing either Nurr1, PitX3, both, or neither. Nurr1 and PitX3 co-transduction led to significantly higher levels of luciferase expression than either factor alone in EB-differentiated mouse ES cultures or COS7 monkey kidney cells (FIG. 32a). Chromatin immunoprecipitation with antibodies to Flag epitope-tagged Nurr1 (FIG. 32b) or untagged PitX3 indicated that these factors bind directly to proximal regions of the endogenous DAT and TH upstream regulatory sequences in vivo.

Sequence analysis of the promoter regions of several mDN-expressed genes, including DAT (FIG. 32c), TyRP-1, and TH, indicated that all of these harbor adjacent putative binding elements for Nurr1 and PitX3, suggesting a cooperative binding strategy. Consistent with this, mutagenesis of these adjacent sites in the human DAT promoter largely abrogated promoter activation by Nurr1 and PitX3 in COS7 cells (FIG. 32d) and MM13 ES cultures. Finally, gel shift assays show that Nurr1 and PitX3 bind cooperatively to oligonucleotides that harbor adjacent putative binding sites from the proximal DAT promoter (FIG. 32e). Mutagenesis of conserved nucleotides within these sites inhibits binding activity. Taken together, these data strongly support the notion that cooperative DNA binding by Nurr1 and PitX3 underlie the induction of mDN maturation genes.

Cooperativity between orphan nuclear receptors and paired-like homeodomain proteins has previously been described in an invertebrate model. The Fushi Tarazu factor 1 (Ftz-F1) protein is an orphan nuclear receptor that is required along with Fush Tarazu (Ftz) for normal embryonic segmentation in Drosophila, and these two factors bind cooperatively to target DNA sequences (Yu et al., 1997; Guichet et al., 1997). The interaction between paired-domain homoeproteins and orphan receptors appears to be conserved in vertebrate species (Steffenson et al., 2004).

Our data, taken together with loss of function gene knockout studies in rodents (Zetterstrom et al., 1997; Saucedo-Cardenas et al., 1998; Castillo et al., 1998; Hwang et al., 2003; Nunes et al., 2003; van den Munckhof et al., 2003; Smidt et al., 2004), indicate that PitX3 and Nurr1 cooperatively induce the late maturation of mDNs. PitX3 and Nurr1 cooperativity offers a potential mechanism for the relatively cell-type specific expression of late markers of mDN maturation. Thus, neither Nurr1 nor PitX3 expression is strictly confined to mDNs: Nurr1 is expressed broadly in the brain during late development, as well as in other tissues, whereas PitX3 is also found in the eye; the region of expression overlap, however, is confined to mDNs.

Expression of both Nurr1 and PitX3 is maintained in adult mDNs, and thus the synergistic activity of these genes may ultimately also regulate mDN survival in vivo. Consistent with this notion, PitX3-deficient animals display a progressive deficit in SN DNs postnatally, although VTA DNs are affected to a lesser extent (Hwang et al., 2003; Nunes et al., 2003; van den Munckhof et al., 2003; Smidt et al., 2004). Furthermore, mDNs of Nurr1 heterozygous animals are reported to display reduced survival (Jiang et al., 2005; Le et al., 1999), and mutations at the human Nurr1 locus may predispose individuals to PD (Le et al., 2003).

In summary, we describe a network of transcription factors that cooperatively promote the maturation of ES cultures to the mDN phenotype. Prior studies have demonstrated that Nurr1 alone can promote an earlier stage in the differentiation of murine ES cultures (Kim et al., 2002; Kim et al., 2005). We extend this work to show that Nurr1 along with PitX3 cooperatively promote the expression of a late maturation phenotype. Also, we demonstrate feasibility of genetic modification of human ES cultures to promote the generation of mDNs. We posit that the co-expression of Nurr1 and PitX3 may significantly enhance the efficacy of cell replacement therapies for PD, and in particular human ES-derived cell approaches.

Generation of DY-1 ES cells. Homozygous ‘knock-in’ mice that harbor the Cre recombinase gene at the DAT locus were bred with mice in which a Cre-inducible eYFP fluorescent marker gene was ‘knocked-in’ at the ROSA26 locus (Srinvas et al., 2001; Zhaung et al., 2001; Chuhma et al., 2004) to obtain double-transgenic blastocysts. An ES cell line (DY-1) was derived from these blastocysts using standard techniques (Wichterle et al., 2002). This cell line was demonstrated to be totipotent by injection into blastocysts and generation of ES-derived chimeric animals that efficiently transmit both transgenes through the germline.

ES cells culture and in vitro differentiation. Mouse ES cells (MM13 or DY-1) were propagated and differentiated as described previously (FIG. 29) (Lee et al. 2000; Martinat et al. 2004). Briefly, to induce EB formation (stage 2), the cells were dissociated to a single-cell suspension by Trypsin (0.025%, Specialty Media) and plated onto nonadherent bacterial dishes at a density of 2-2.5×10₄ cells/cm2 in ES media without Leukemia Inhibitory factor (LIF). Four-day-old EBs were plated onto an adherent tissue culture surface in ES medium for one day. The selection of nestin-positive cells (stage 3) was initiated by replacing the ES medium with a serum-free insulin/transferring/selenium/fibronectin (ITSF) medium (Okabe et al., 1996). After 6-10 days of selection, cells were dissociated by Trypsin (0.025%, Specialty Media) and plated on tissue culture dishes or glass coverslips at a concentration of 1-1.5×10⁵ cells/cm2 in N2 medium (Johe et al., 1996) containing 1 μg/ml of laminin (Sigma) and 10 ng/ml of basic Fibroblast growth factor (bFGF; InVitroGen). Before cell plating, tissue surface and glass coverslips were precoated with polyornithine (6 μg/ml, Sigma) and laminin (3 μg/ml, Sigma). Neural precursors were expanded for 6-8 days (stage 4). Terminal differentiation (stage 5) was induced by the withdrawal of bFGF in N2 medium supplemented with 200 μM ascorbic acid (AA; Sigma) for 6-10 days.

Human ES cell line H9 (passages 30-45) was cultured on mitotically inactivated mouse embryonic fibroblasts (MEF, Specialty Media). Undifferentiated hES cells were maintained under growth conditions and passaging techniques as previously described (Perrier et al., 2004). Differentiation of hES cells into dopamine neurons was performed using the SDIA method (Perrier et al, 2004). Briefly, neural induction was achieved by plating hES cells (1×10³ cells/cm²) on a confluent layer of mitotically inactivated stromal feeder cells (MS5) in knock-out serum replacement (KSR) medium. Neural patterning occurred in the presence of 200 ng/ml Shh, 100 ng/ml FGF8b, 20 ng/ml BDNF and 20 ng/ml GDNF (all the factors are from R&D system). At passages 1 and 2, hEScells-derived rosettes were plated on a 15 μg/ml polyornithine and 1 ∝g/ml laminin coated substrate in the absence of feeder cells. Neural differentiation is induced by the withdrawal of Shh/FGF8b. To increase the dopaminergic differentiation and survival, cells were exposed to 200 μM AA, 20 ng/ml BDNF, 20 ng/ml GDNF and 1 ng/ml transforming growth factor type β3 (all the factors are from R&D system).

Recombinant lentiviral vectors. The recombinant pTRIP lentiviral vectors have been described (Zennou et al., 2001). Nurr1 and PitX3 cDNAs were cloned by standard PCR methods from a human cDNA library, and the cDNAs were inserted into the pTRIP vector under the regulation of the EF1α Promoter (Martinat et al., 2004). Lmx1b was cloned from a chick cDNA library. Viral particles were pseudotyped with the vesicular stomatitis virus G-protein and produced as described (Naldini et al., 1996; Starpoli et al., 2003). For the viral transduction, single cell-suspensions (1×10⁵ cells) were infected at a multiplicity of infection of 1:1.

Immunocytochemistry and In Situ hybridization. Cultured cells were fixed in 4% paraformaldehyde in PBS and were incubated with the primary antibodies at 4° C. for 12 h. The following primary antibodies were used: mouse anti-TuJ1 (Covance, dilution 1/500), rabbit anti-TH (Pel-Freez, dilution 1/1000), sheep anti-TH (Pel-Freez, dilution 1/1000), rat anti-DAT (Chemicon, dilution 1/500), rabbit anti-GABA (Chemicon, dilution 1/1000), rabbit anti-5HT (Sigma, dilution 1/5000), and rabbit anti-GFP (Molecular probes, dilution 1/1000). Appropriate FITC, Cy3 and Cy5-labeled secondary antibodies (Jackson ImmunoResearch) were used.

In situ hybridization were performed using Lmx1b and Engrailed-1 probes as previously described Kania et al., 2000).

Quantitative real-time PCR. Total RNA from ES cells differentiated was prepared using the Absolutely RNA Miniprep kit (Stratagene). cDNA was synthesized using the SuperScript first strand synthesis system for RT-PCR (Invitrogen) with random hexamers. Real-time PCR reactions were optimized to determine the linear amplification range. Quantitative real-time RT-PCRs were performed in Stratagene MX3000P using the QuantiTect SYBR Green PCR Master Mix (Qiagen) according to the manufacturer's instructions. For quantification analyses, PCR products levels were measured in real time during the annealing step, and values were normalized to those of β-actin. Primer sequences are provided in Table 1.

HPLC analysis. Extracellular dopamine levels were measured by HPLC as previously described (Martinat et al., 2004). Briefly, cells were treated with 100 uM of L-DOPA for 1 hour at 37° C. and then stimulated with high KCl media (56 mM) for 15 min at 37° C. Media were collected and a solution of 0.2 M perchloric acid (PCA) was added to a final concentration of 0.1 M PCA. These samples were centrifuged and analyzed by HPLC-EC on an ESA Coulochem HPLC. The results were normalized to the amount of protein as determined using the BCA protein assay reagent (Pierce).

6-OHDA lesioning, transplantation, and behavioral analysis. Procedures involving animal care were in conformity with the Columbia University Animal Protocols that are in compliance with the guidelines of the National Institute of Health. Adult male CD-1 mice (6-8 weeks; Charles River Laboratories) were housed at a constant temperature (23° C.) with a fixed 12 hrs light/dark cycle and had ad libidum access to food and water. Animals were anaesthetized with Ketamine and Xylazine (60 mg/kg and 10 mg/kg, respectively) and placed in a stereotactic frame (Stoelting). The dopamine denervation was achieved by streotactic injection of 6-OHDA (4 μl; 2 mg/ml in normal saline with 0.02% ascorbic acid; Sigma) in the left striatum (anterior 1 mm; lateral 2.2 mm; ventral 3 mm as determined from the bregma and the skull surface). The 6-OHDA solution was infused at the rate of 0.5 μl/min using a 33-gauge Hamilton microsyringe. The needle was left in position for an additional 5 min before removal. Stage 3 EB-differentiated human or mouse ES cells dissociated with trypsin (Invitrogen), transduced with GFP or Nurr1/PitX3/GFP lentiviruses, washed twice with PBS, and resuspended in DMEM-F12 media (InVitroGen). 2 μl of the cell suspension (1×10⁵ cells/μl) was injected in the striatum (performed as for 6-OHDA injection). Apomorphine-induced turning behavior was assessed at two weeks after the 6-OHDA injection and prior to grafting, and again 6 weeks after the cell grafting (Nishimura et al., 2003). Mice were placed in hemispheric bowls and left for 20 min to habituate to the new environment. Apomorphine was injected subcutaneously (0.4 mg/kg). Mice were videotaped and the number of turns was counted during a 30 min period by an independent observer blinded to the experimental design. Data were analyzed by the Fisher test (Anova) using Statview software. One animal that had been transplanted with control vector-transduced mouse ES cells displayed severe contralateral barrel rotations in response to 0.4 mg/kg apomorphine (consistent with the apparent low efficacy of the control cells), but these could not be accurately quantified in this assay and this animal was therefore not included in the statistical analysis. Additionally, in two animals transplanted with human ES cells (1 GFP and 1 Nurr1/PitX3), the stereotaxic surgery was not successful and no engraftment was visualized by GFP fluorescence; these were not included in further analyses.

Six weeks after transplantation, animals were sacrificed and perfused with 10 ml of saline followed by 35 ml of a 4% solution of paraformaldehyde (PFA) and maintained at 4° C. Brains were extracted and left for an additional 24 h in PFA 4% at 4° C. Using a vibratome (Leica), 40 μm-thick frontal sections were cut through the entire anteroposterior axis of the striatum. Immunostaining and microscopy on slices were performed as described for the cultured cells (Martinat et al., 2004). Transplanted human cells were identified by immunostaining with a mouse monoclonal antibody specific for human nuclear antigen (Chemicon, 1:100 dilution).

At late time points (8 weeks or longer after cell grafting), 3 of the mice that received mouse ES cell transplants developed apparent teratoma-like masses at the implantation site (1 transplanted with Nurr1 and PitX3-transduced cells, 2 with control virus-transduced cells), consistent with previous reports (Bjorklund et al., 2002). This likely reflects the presence of a population of immature cells in these mouse cultures. Importantly, no teratomas were observed with the human ES cell transplantations.

Luciferase assays, chromatin immunoprecipitations, and gel shift analysis. For Luciferase assays, cells were transfected using Lipofectamine (Invitrogen) with a firefly luciferase assay plasmid harboring either DAT regulatory sequences25 or control. 48 h after transfection, cell extracts were analysed for firefly luciferase activity and normalized for renilla luciferase activity (Promega), following manufacturer's instructions.

ChIP analysis was carried out as described47, with minor modifications. DNA-protein complexes were immunoprecipitated utilizing agarose conjugated anti-Flag antibodies (Sigma). The sequences of the primers are for actin promoter 5′-AATACTGTGTACTCTCAAGATGGA-3′ (SEQ ID NO:8) and 5′-TTACGCCTAGCGTGTAGACTCT-3′ (SEQ ID NO:9), for TH promoter 5′-CAATTAGATCTAATGGGACGGAG-3′ (SEQ ID NO:10) and 5′-TGCTCTGAGAGGGGTCTTCTGAAG-3′ (SEQ ID NO:11), for DAT promoter 5′-TCTATAACCTTGCAAAGACACCT-3′ (SEQ ID NO:12) and 5′-AGAAGCGATCTTGGTCCTTACCTG-3′ (SEQ ID NO:13).

Electrophoretic mobility shift assays (EMSA) were performed using an EMSA kit (Novagen) as per the manufacturer's instructions with annealed oligonucleotides for wild-type human DAT promoter sequences (5′-GTC TAC TGG ATA AGA GCC CGA GGC CGA GGC TGA GAC CG-3′ (SEQ ID NO:14) and 5′-CTC AGC CTC GGC CTC GGG CTC TTA TCC AGT AGA CAG GGT-3′ (SEQ ID NO:15)) or mutant human DAT promoter sequences (5′-GTC TAC TGG ATG GGA GCC CGA TTC CGA GGC TGA GAC CG-3′ (SEQ ID NO:16) and 5′-CTC AGC CTC GGA ATC GGG CTC CCA TCC AGT AGA CAG GGT-3′ (SEQ ID NO:17)).

### REFERENCES

The content of the following references are incorporated into the foregoing specification in their entirety as fully as though set forth therein as background for those skilled in the art.

Abeliovich A, Schmitz Y, Farinas I, Choi-Lundberg D, Ho W H, et al. (2000) Mice lacking alpha-synuclein display functional deficits in the nigrostriatal dopamine system. Neuron 25(1): 239-252.

Abeliovich, A., Paylor, R., Chen, C., Kim, J. J., Wehner, J. M., and Tonegawa, S. (1993). PKC gamma mutant mice exhibit mild deficits in spatial and contextual learning. Cell 75, 1263-1271.

Abeliovich, A., Schmitz, Y., Farinas, I., Choi-Lundberg, D., Ho, W. H., Castillo, P. E., Shinsky, N., Verdugo, J. M., Armanini, M., Ryan, A., et al. (2000). Mice lacking alpha-synuclein display functional deficits in the nigrostriatal dopamine system. Neuron 25, 239-252.

Alberi, L., Sgado, P. & Simon, H. H. Engrailed genes are cell-autonomously required to prevent apoptosis in mesencephalic dopaminergic neurons. Development 131, 3229-36 (2004).

Angelastro, J. M., Ryu, E. J., Torocsik, B., Fiske, B. K., and Greene, L. A. (2002a). Blue-white selection step enhances the yield of SAGE concatemers. Biotechniques 32, 484, 486.

Angelastro, J. M., Torocsik, B., and Greene, L. A. (2002b). Nerve growth factor selectively regulates expression of transcripts encoding ribosomal proteins. BMC Neurosci 3, 3.

Bagri, A., Marin, O., Plump, A. S., Mak, J., Pleasure, S. J., Rubenstein, J. L., and Tessier-Lavigne, M. (2002). Slit proteins prevent midline crossing and determine the dorsoventral position of major axonal pathways in the mammalian forebrain. Neuron 33, 233-248.

Barberi T, Klivenyi P, Calingasan N Y, Lee H, Kawamata H, et al. (2003) Neural subtype specification of fertilization and nuclear transfer embryonic stem cells and application in parkinsonian mice. Nat Biotechnol 21(10): 1200-1207.

Barberi, T., Klivenyi, P., Calingasan, N. Y., Lee, H., Kawamata, H., Loonam, K., Perrier, A. L., Bruses, J., Rubio, M. E., Topf, N., et al. (2003). Neural subtype specification of fertilization and nuclear transfer embryonic stem cells and application in parkinsonian mice. Nat Biotechnol 21, 1200-1207.

Beck, K. D., Irwin, I., Valverde, J., Brennan, T. J., Langston, J. W., and Hefti, F. (1996). GDNF induces a dystonia-like state in neonatal rats and stimulates dopamine and serotonin synthesis. Neuron 16, 665-673.

Beck, K. D., Knusel, B., and Hefti, F. (1993). The nature of the trophic action of brain-derived neurotrophic factor, des(1-3)-insulin-like growth factor-1, and basic fibroblast growth factor on mesencephalic dopaminergic neurons developing in culture. Neuroscience 52, 855-866.

Berezovska, O., McLean, P., Knowles, R., Frosh, M., Lu, F. M., Lux, S. E., and Hyman, B. T. (1999). Notch1 inhibits neurite outgrowth in postmitotic primary neurons. Neuroscience 93, 433-439.

Bian K, Gao Z, Weisbrodt N, Murad F (2003) The nature of heme/iron-induced protein tyrosine nitration. Proc Natl Acad Sci USA 100(10): 5712-5717.

Bixby, S., Kruger, G. M., Mosher, J. T., Joseph, N. M., and Morrison, S. J. (2002). Cell-intrinsic differences between stem cells from different regions of the peripheral nervous system regulate the generation of neural diversity. Neuron 35, 643-656.

Bjorklund, L. M., Sanchez-Pernaute, R., Chung, S., Andersson, T., Chen, I. Y., McNaught, K. S., Brownell, A. L., Jenkins, B. G., Wahlestedt, C., Kim, K. S., and Isacson, O. (2002). Embryonic stem cells develop into functional dopaminergic neurons after transplantation in a Parkinson rat model. Proc Natl Acad Sci USA 99, 2344-2349.

Bonifati V, Rizzu P, van Baren M J, Schaap O, Breedveld G J, et al. (2003) Mutations in the DJ-1 gene associated with autosomal recessive early-onset parkinsonism. Science 299(5604): 256-259.

Brunet, J. F., and Pattyn, A. (2002). Phox2 genes—from patterning to connectivity. Curr Opin Genet Dev 12, 435-440.

Buonanno, A., and Fischbach, G. D. (2001). Neuregulin and ErbB receptor signaling pathways in the nervous system. Curr Opin Neurobiol 11, 287-296.

Burke, R. E. (2003). Postnatal developmental programmed cell death in dopamine neurons. Ann N Y Acad Sci 991, 69-79.

Castelo-Branco, G., Wagner, J., Rodriguez, F. J., Kele, J., Sousa, K., Rawal, N., Pasolli, H. A., Fuchs, E., Kitajewski, J., and Arenas, E. (2003). Differential regulation of midbrain dopaminergic neuron development by Wnt-1, Wnt-3a, and Wnt-5a. Proc Natl Acad Sci USA 100, 12747-12752.

Castillo, S. O. et al. Dopamine biosynthesis is selectively abolished in substantia nigra/ventral tegmental area but not in hypothalamic neurons in mice with targeted disruption of the Nurr1 gene. Mol Cell Neurosci 11, 36-46 (1998).

Cheng, L., Chen, C. L., Luo, P., Tan, M., Qiu, M., Johnson, R., and Ma, Q. (2003). Lmx1b, Pet-1, and Nkx2.2 coordinately specify serotonergic neurotransmitter phenotype. J Neurosci 23, 9961-9967.

Chuhma, N. et al. Dopamine neurons mediate a fast excitatory signal via their glutamatergic synapses. J Neurosci 24, 972-81 (2004).

Chung, S., Sonntag, K. C., Andersson, T., Bjorklund, L. M., Park, J. J., Kim, D. W., Kang, U. J., Isacson, O., and Kim, K. S. (2002). Genetic engineering of mouse embryonic stem cells by Nurr1 enhances differentiation and maturation into dopaminergic neurons. Eur J Neurosci 16, 1829-1838.

Chung, S. et al. The homeodomain transcription factor Pitx3 facilitates differentiation of mouse embryonic stem cells into AHD2-expressing dopaminergic neurons. Mol Cell Neurosci 28, 241-52 (2005).

Dasen, J. S., Liu, J. P., and Jessell, T. M. (2003). Motor neuron columnar fate imposed by sequential phases of Hox-c activity. Nature 425, 926-933.

Dauer W, Przedborski S (2003) Parkinson's disease: mechanisms and models. Neuron 39(6): 889-909.

Dauer, W., and Przedborski, S. (2003). Parkinson's disease: mechanisms and models. Neuron 39, 889-909.

de Nobel H, Lawrie L, Brul S, Klis F, Davis M, et al. (2001) Parallel and comparative analysis of the proteome and transcriptome of sorbic acid-stressed Saccharomyces cerevisiae. Yeast 18(15): 1413-1428.

Demasi M, Davies K J (2003) Proteasome inhibitors induce intracellular protein aggregation and cell death by an oxygen-dependent mechanism. FEBS Lett 542(1-3): 89-94.

Dubreuil, V., Hirsch, M. R., Jouve, C., Brunet, J. F., and Goridis, C. (2002). The role of Phox2b in synchronizing pan-neuronal and type-specific aspects of neurogenesis. Development 129, 5241-5253.

Farrer M, Wavrant-De Vrieze F, Crook R, Boles L, Perez-Tur J, et al. (1998) Low frequency of alpha-synuclein mutations in familial Parkinson's disease. Ann Neurol 43(3): 394-397.

Feng, L., Wang, C. Y., Jiang, H., Oho, C., Mizuno, K., Dugich-Djordjevic, M., and Lu, B. (1999). Differential effects of GDNF and BDNF on cultured ventral mesencephalic neurons. Brain Res Mol Brain Res 66, 62-70.

Fezoui Y, Hartley D M, Walsh D M, Selkoe D J, Osterhout J J, et al. (2000) A de novo designed helix-turn-helix peptide forms nontoxic amyloid fibrils. Nat Struct Biol 7(12): 1095-1099.

Floss T, Wurst W (2002) Functional genomics by gene-trapping in embryonic stem cells. Methods Mol Biol 185: 347-379.

Freed, C. R. (2002). Will embryonic stem cells be a useful source of dopamine neurons for transplant into patients with Parkinson's disease? Proc Natl Acad Sci USA 99, 1755-1757.

Freed, C. R., Breeze, R. E., and Fahn, S. (2000). Placebo surgery in trials of therapy for Parkinson's disease. N Engl J Med 342, 353-354; author reply 354-355.

Giasson B I, Duda J E, Quinn S M, Zhang B, Trojanowski J Q, et al. (2002) Neuronal alpha-synucleinopathy with severe movement disorder in mice expressing A53T human alpha-synuclein. Neuron 34(4): 521-533.

Gobeil S, Boucher C C, Nadeau D, Poirier G G (2001) Characterization of the necrotic cleavage of poly(ADP-ribose) polymerase (PARP-1): implication of lysosomal proteases. Cell Death Differ 8(6): 588-594.

Goda, Y., and Davis, G. W. (2003). Mechanisms of synapse assembly and disassembly. Neuron 40, 243-264.

Goldberg M S, Fleming S M, Palacino J J, Cepeda C, Lam H A, et al. (2003) Parkin-deficient mice exhibit nigrostriatal deficits but not loss of dopaminergic neurons. J Biol Chem 278(44): 43628-43635.

Goldberg M S, Lansbury P T, Jr. (2000) Is there a cause-and-effect relationship between alpha-synuclein fibrillization and Parkinson's disease? Nat Cell Biol 2(7): E115-119.

Goridis, C., and Rohrer, H. (2002). Specification of catecholaminergic and serotonergic neurons. Nat Rev Neurosci 3, 531-541.

Guichet, A. et al. The nuclear receptor homologue Ftz-F1 and the homeodomain protein Ftz are mutually dependent cofactors. Nature 385, 548-52 (1997).

Hague S, Rogaeva E, Hernandez D, Gulick C, Singleton A, et al. (2003) Early-onset Parkinson's disease caused by a compound heterozygous DJ-1 mutation. Ann Neurol 54(2): 271-274.

Han B S, Hong H S, Choi W S, Markelonis G J, Oh T H, et al. (2003) Caspase-dependent and-independent cell death pathways in primary cultures of mesencephalic dopaminergic neurons after neurotoxin treatment. J Neurosci 23(12): 5069-5078.

Hattori N, Shimura H, Kubo S, Kitada T, Wang M, et al. (2000) Autosomal recessive juvenile parkinsonism: a key to understanding nigral degeneration in sporadic Parkinson's disease. Neuropathology 20 Suppl: S85-90.

Heid, C. A., Stevens, J., Livak, K. J., and Williams, P. M. (1996). Real time quantitative PCR. Genome Res 6, 986-994.

Hemmendinger, L. M., Garber, B. B., Hoffmann, P. C., and Heller, A. (1981). Target neuron-specific process formation by embryonic mesencephalic dopamine neurons in vitro. Proc Natl Acad Sci USA 78, 1264-1268.

Hwang, D. Y., Ardayfio, P., Kang, U. J., Semina, E. V. & Kim, K. S. Selective loss of dopaminergic neurons in the substantia nigra of Pitx3-deficient aphakia mice. Brain Res Mol Brain Res 114, 123-31 (2003).

Itier J M, Ibanez P, Mena M A, Abbas N, Cohen-Salmon C, et al. (2003) Parkin gene inactivation alters behaviour and dopamine neurotransmission in the mouse. Hum Mol Genet 12(18): 2277-2291.

Jenner P (2003) Oxidative stress in Parkinson's disease. Ann Neurol 53 Suppl 3: S26-36; discussion S36-28.

Jenner P, Olanow C W (1998) Understanding cell death in Parkinson's disease. Ann Neurol 44(3 Suppl 1): S72-84.

Jiang, C. et al. Age-dependent dopaminergic dysfunction in Nurr1 knockout mice. Exp Neurol 191, 154-62 (2005).

Johe, K. K., Hazel, T. G., Muller, T., Dugich-Djordjevic, M. M. & McKay, R. D. Single factors direct the differentiation of stem cells from the fetal and adult central nervous system. Genes Dev 10, 3129-40 (1996).

Johnson, R. A., Eshleman, A. J., Meyers, T., Neve, K. A., and Janowsky, A. (1998). [3H]substrate- and cell-specific effects of uptake inhibitors on human dopamine and serotonin transporter-mediated efflux. Synapse 30, 97-106.

Kania, A., Johnson, R. L. & Jessell, T. M. Coordinate roles for LIM homeobox genes in directing the dorsoventral trajectory of motor axons in the vertebrate limb. Cell 102, 161-73 (2000).

Kawasaki H, Mizuseki K, Nishikawa S, Kaneko S, Kuwana Y, et al. (2000) Induction of midbrain dopaminergic neurons from ES cells by stromal cell-derived inducing activity. Neuron 28(1): 31-40.

Kawasaki, H., Mizuseki, K., and Sasai, Y. (2002a). Selective neural induction from ES cells by stromal cell-derived inducing activity and its potential therapeutic application in Parkinson's disease. Methods Mol Biol 185, 217-227.

Kawasaki, H., Mizuseki, K., Nishikawa, S., Kaneko, S., Kuwana, Y., Nakanishi, S., Nishikawa, S. I., and Sasai, Y. (2000). Induction of midbrain dopaminergic neurons from ES cells by stromal cell-derived inducing activity. Neuron 28, 31-40.

Kawasaki, H., Suemori, H., Mizuseki, K., Watanabe, K., Urano, F., Ichinose, H., Haruta, M., Takahashi, M., Yoshikawa, K., Nishikawa, S., et al. (2002b). Generation of dopaminergic neurons and pigmented epithelia from primate ES cells by stromal cell-derived inducing activity. Proc Natl Acad Sci USA 99, 1580-1585.

Kim, J. H., Auerbach, J. M., Rodriguez-Gomez, J. A., Velasco, I., Gavin, D., Lumelsky, N., Lee, S. H., Nguyen, J., Sanchez-Pernaute, R., Bankiewicz, K., and McKay, R. (2002). Dopamine neurons derived from embryonic stem cells function in an animal model of Parkinson's disease. Nature 418, 50-56.

Kim, J. Y., Koh, H. C., Lee, J. Y., Chang, M. Y., Kim, Y. C., Chung, H. Y., Son, H., Lee, Y. S., Studer, L., McKay, R., and Lee, S. H. (2003). Dopaminergic neuronal differentiation from rat embryonic neural precursors by Nurr1 overexpression. J Neurochem 85, 1443-1454.

Kim, D. W. et al. Stromal cell-derived inducing activity, Nurr1 and signaling molecules synergistically induce dopaminergic neurons from mouse embryonic stem cells. Stem Cells (2005).

Kues, W. A., Anger, M., Carnwath, J. W., Paul, D., Motlik, J., and Niemann, H. (2000). Cell cycle synchronization of porcine fetal fibroblasts: effects of serum deprivation and reversible cell cycle inhibitors. Biol Reprod 62, 412-419.

Le, W. D., Xu, P., Jankovic, J., Jiang, H., Appel, S. H., Smith, R. G., and Vassilatis, D. K. (2003). Mutations in NR4A2 associated with familial Parkinson disease. Nat Genet 33, 85-89.

Le, W., Conneely, O. M., Zou, L., He, Y., Saucedo-Cardenas, O., Jankovic, J., Mosier, D. R., and Appel, S. H. (1999). Selective agenesis of mesencephalic dopaminergic neurons in Nurr1-deficient mice. Exp Neurol 159, 451-458.

Lee M K, Stirling W, Xu Y, Xu X, Qui D, et al. (2002) Human alpha-synuclein-harboring familial Parkinson's disease-linked Ala-53→Thr mutation causes neurodegenerative disease with alpha-synuclein aggregation in transgenic mice. Proc Natl Acad Sci USA 99(13): 8968-8973.

Lee, S. H., Lumelsky, N., Studer, L., Auerbach, J. M., and McKay, R. D. (2000). Efficient generation of midbrain and hindbrain neurons from mouse embryonic stem cells. Nat Biotechnol 18, 675-679.

Maira, M., Martens, C., Philips, A., and Drouin, J. (1999). Heterodimerization between members of the Nur subfamily of orphan nuclear receptors as a novel mechanism for gene activation. Mol Cell Biol 19, 7549-7557.

Martinat, C. et al. Sensitivity to oxidative stress in DJ-1-deficient dopamine neurons: an ES-derived cell model of primary parkinsonism. PLoS Biol 2, e327 (2004).

Masliah E, Rockenstein E, Veinbergs I, Mallory M, Hashimoto M, et al. (2000) Dopaminergic loss and inclusion body formation in alpha-synuclein mice: implications for neurodegenerative disorders. Science 287(5456): 1265-1269.

McCormack A L, Thiruchelvam M, Manning-Bog A B, Thiffault C, Langston J W, et al. (2002) Environmental risk factors and Parkinson's disease: selective degeneration of nigral dopaminergic neurons caused by the herbicide paraquat. Neurobiol Dis 10(2): 119-127.

Miranda, M., Botti, D., Bonfigli, A., Ventura, T. & Arcadi, A. Tyrosinase-like activity in normal human substantia nigra. Gen Pharmacol 15, 541-4 (1984).

Mitsumoto A, Nakagawa Y (2001) DJ-1 is an indicator for endogenous reactive oxygen species elicited by endotoxin. Free Radic Res 35(6): 885-893.

Mitsumoto A, Nakagawa Y, Takeuchi A, Okawa K, Iwamatsu A, et al. (2001) Oxidized forms of peroxiredoxins and DJ-1 on two-dimensional gels increased in response to sublethal levels of paraquat. Free Radic Res 35(3): 301-310.

Mizushima N, Yamamoto A, Hatano M, Kobayashi Y, Kabeya Y, et al. (2001) Dissection of autophagosome formation using Apg5-deficient mouse embryonic stem cells. J Cell Biol 152(4): 657-668.

Morizane A, Takahashi J, Takagi Y, Sasai Y, Hashimoto N (2002) Optimal conditions for in vivo induction of dopaminergic neurons from embryonic stem cells through stromal cell-derived inducing activity. J Neurosci Res 69(6): 934-939.

Morizane, A., Takahashi, J., Takagi, Y., Sasai, Y., and Hashimoto, N. (2002). Optimal conditions for in vivo induction of dopaminergic neurons from embryonic stem cells through stromal cell-derived inducing activity. J Neurosci Res 69, 934-939.

Nagakubo D, Taira T, Kitaura H, Ikeda M, Tamai K, et al. (1997) DJ-1, a novel oncogene which transforms mouse NIH3T3 cells in cooperation with ras. Biochem Biophys Res Commun 231(2): 509-513.

Naldini, L. et al. In vivo gene delivery and stable transduction of nondividing cells by a lentiviral vector. Science 272, 263-7 (1996).

Nishimura, F. et al. Potential use of embryonic stem cells for the treatment of mouse parkinsonian models: improved behavior by transplantation of in vitro differentiated dopaminergic neurons from embryonic stem cells. Stem Cells 21, 171-80 (2003).

Nunes, I., Tovmasian, L. T., Silva, R. M., Burke, R. E., and Goff, S. P. (2003). Pitx3 is required for development of substantia nigra dopaminergic neurons. Proc Natl Acad Sci USA 100, 4245-4250.

Odom, D. T. et al. Control of pancreas and liver gene expression by HNF transcription factors. Science 303, 1378-81 (2004).

Okabe, S., Forsberg-Nilsson, K., Spiro, A. C., Segal, M. & McKay, R. D. Development of neuronal precursor cells and functional postmitotic neurons from embryonic stem cells in vitro. Mech Dev 59, 89-102 (1996).

Olanow, C. W., Goetz, C. G., Kordower, J. H., Stoessl, A. J., Sossi, V., Brin, M. F., Shannon, K. M., Nauert, G. M., Perl, D. P., Godbold, J., and Freeman, T. B. (2003). A double-blind controlled trial of bilateral fetal nigral transplantation in Parkinson's disease. Ann Neurol 54, 403-414.

Packard, M., Mathew, D., and Budnik, V. (2003). Wnts and TGF beta in synaptogenesis: old friends signalling at new places. Nat Rev Neurosci 4, 113-120.

Pattyn, A., Vallstedt, A., Dias, J. M., Samad, O. A., Krumlauf, R., Rijli, F. M., Brunet, J. F., and Ericson, J. (2003). Coordinated temporal and spatial control of motor neuron and serotonergic neuron generation from a common pool of CNS progenitors. Genes Dev 17, 729-737.

Perrier, A. L. et al. Derivation of midbrain dopamine neurons from human embryonic stem cells. Proc Natl Acad Sci USA 101, 12543-8 (2004).

Perrone-Capano, C., Da Pozzo, P., and di Porzio, U. (2000). Epigenetic cues in midbrain dopaminergic neuron development. Neurosci Biobehav Rev 24, 119-124.

Polymeropoulos M H, Lavedan C, Leroy E, Ide S E, Dehejia A, et al. (1997) Mutation in the alpha-synuclein gene identified in families with Parkinson's disease. Science 276(5321): 2045-2047.

Pothos E, Desmond M, Sulzer D (1996) L-3,4-dihydroxyphenylalanine increases the quantal size of exocytotic dopamine release in vitro. J Neurochem 66(2): 629-636.

Pothos, E. N., Davila, V., and Sulzer, D. (1998). Presynaptic recording of quanta from midbrain dopamine neurons and modulation of the quantal size. J Neurosci 18, 4106-4118.

Prochiantz, A., di Porzio, U., Kato, A., Berger, B., and Glowinski, J. (1979). In vitro maturation of mesencephalic dopaminergic neurons from mouse embryos is enhanced in presence of their striatal target cells. Proc Natl Acad Sci USA 76, 5387-5391.

Rideout H J, Larsen K E, Sulzer D, Stefanis L (2001) Proteasomal inhibition leads to formation of ubiquitin/alpha-synuclein-immunoreactive inclusions in PC12 cells. J Neurochem 78(4): 899-908.

Rolletschek, A., Chang, H., Guan, K., Czyz, J., Meyer, M., and Wobus, A. M. (2001). Differentiation of embryonic stem cell-derived dopaminergic neurons is enhanced by survival-promoting factors. Mech Dev 105, 93-104.

Rubinson D A, Dillon C P, Kwiatkowski A V, Sievers C, Yang L, et al. (2003) A lentivirus-based system to functionally silence genes in primary mammalian cells, stem cells and transgenic mice by RNA interference. Nat Genet 33(3): 401-406.

Rubinson, D. A., Dillon, C. P., Kwiatkowski, A. V., Sievers, C., Yang, L., Kopinja, J., Rooney, D. L., Ihrig, M. M., McManus, M. T., Gertler, F. B., et al. (2003). A lentivirus-based system to functionally silence genes in primary mammalian cells, stem cells and transgenic mice by RNA interference. Nat Genet 33, 401-406.

Sacchetti, P., Mitchell, T. R., Granneman, J. G. & Bannon, M. J. Nurr1 enhances transcription of the human dopamine transporter gene through a novel mechanism. J Neurochem 76, 1565-72 (2001).

Saha, S., Bardelli, A., Buckhaults, P., Velculescu, V. E., Rago, C., St Croix, B., Romans, K. E., Choti, M. A., Lengauer, C., Kinzler, K. W., and Vogelstein, B. (2001). A phosphatase associated with metastasis of colorectal cancer. Science 294, 1343-1346.

Sakurada, K., Ohshima-Sakurada, M., Palmer, T. D., and Gage, F. H. (1999). Nurr1, an orphan nuclear receptor, is a transcriptional activator of endogenous tyrosine hydroxylase in neural progenitor cells derived from the adult brain. Development 126, 4017-4026.

Saucedo-Cardenas, O. et al. Nurr1 is essential for the induction of the dopaminergic phenotype and the survival of ventral mesencephalic late dopaminergic precursor neurons. Proc Natl Acad Sci USA 95, 4013-8 (1998).

Sherer T B, Betarbet R, Stout A K, Lund S, Baptista M, et al. (2002) An in vitro model of Parkinson's disease: linking mitochondrial impairment to altered alpha-synuclein metabolism and oxidative damage. J Neurosci 22(16): 7006-7015.

Simon, H. H., Saueressig, H., Wurst, W., Goulding, M. D. & O'Leary, D. D. Fate of midbrain dopaminergic neurons controlled by the engrailed genes. J Neurosci 21, 3126-34 (2001).

Smidt, M. P., Asbreuk, C. H., Cox, J. J., Chen, H., Johnson, R. L., and Burbach, J. P. (2000). A second independent pathway for development of mesencephalic dopaminergic neurons requires Lmx1b. Nat Neurosci 3, 337-341.

Smidt, M. P. et al. Early developmental failure of substantia nigra dopamine neurons in mice lacking the homeodomain gene Pitx3. Development 131, 1145-55 (2004).

Sonntag, K. C., Simantov, R., Kim, K. S. & Isacson, O. Temporally induced Nurr1 can induce a non-neuronal dopaminergic cell type in embryonic stem cell differentiation. Eur J Neurosci 19, 1141-52 (2004).

Spillantini M G, Crowther R A, Jakes R, Cairns N J, Lantos P L, et al. (1998) Filamentous alpha-synuclein inclusions link multiple system atrophy with Parkinson's disease and dementia with Lewy bodies. Neurosci Lett 251(3): 205-208.

Srinivas, S., Watanabe, T., Lin, C. S., William, C. M., Tanabe, Y., Jessell, T. M., and Costantini, F. (2001). Cre reporter strains produced by targeted insertion of EYFP and ECFP into the ROSA26 locus. BMC Dev Biol 1, 4.

Staropoli J F, McDermott C, Martinat C, Schulman B, Demireva E, et al. (2003) Parkin Is a Component of an SCF-like Ubiquitin Ligase Complex and Protects Postmitotic Neurons from Kainate Excitotoxicity. Neuron 37(5): 735-749.

Staropoli, J. F., McDermott, C., Martinat, C., Schulman, B., Demireva, E., and Abeliovich, A. (2003). Parkin is a component of an SCF-like ubiquitin ligase complex and protects postmitotic neurons from kainate excitotoxicity. Neuron 37, 735-749.

Steffensen, K. R. et al. Functional conservation of interactions between a homeodomain cofactor and a mammalian FTZ-F1 homologue. EMBO Rep 5, 613-9 (2004).

Taira T, Saito Y, Niki T, Iguchi-Ariga S M, Takahashi K, et al. (2004) DJ-1 has a role in antioxidative stress to prevent cell death. EMBO Rep 5(2): 213-218.

Takagi, Y. et al. Dopaminergic neurons generated from monkey embryonic stem cells function in a Parkinson primate model. J Clin Invest 115, 102-9 (2005).

Tornqvist, N., Hermanson, E., Perlmann, T., and Stromberg, I. (2002). Generation of tyrosine hydroxylase-immunoreactive neurons in ventral mesencephalic tissue of Nurr1 deficient mice. Brain Res Dev Brain Res 133, 37-47.

Trojanowski J Q, Lee V M (1998) Aggregation of neurofilament and alpha-synuclein proteins in Lewy bodies: implications for the pathogenesis of Parkinson disease and Lewy body dementia. Arch Neurol 55(2): 151-152.

van den Munckhof, P., Luk, K. C., Ste-Marie, L., Montgomery, J., Blanchet, P. J., Sadikot, A. F., and Drouin, J. (2003). Pitx3 is required for motor activity and for survival of a subset of midbrain dopaminergic neurons. Development 130, 2535-2542.

Velculescu, V. E., Madden, S. L., Zhang, L., Lash, A. E., Yu, J., Rago, C., Lal, A., Wang, C. J., Beaudry, G. A., Ciriello, K. M., et al. (1999). Analysis of human transcriptomes. Nat Genet 23, 387-388.

Wagner, J., Akerud, P., Castro, D. S., Holm, P. C., Canals, J. M., Snyder, E. Y., Perlmann, T., and Arenas, E. (1999). Induction of a midbrain dopaminergic phenotype in Nurr1-overexpressing neural stem cells by type 1 astrocytes. Nat Biotechnol 17, 653-659.

Wallen, A. A., Castro, D. S., Zetterstrom, R. H., Karlen, M., Olson, L., Ericson, J., and Perlmann, T. (2001). Orphan nuclear receptor Nurr1 is essential for Ret expression in midbrain dopamine neurons and in the brain stem. Mol Cell Neurosci 18, 649-663.

Wallen, A., and Perlmann, T. (2003). Transcriptional control of dopamine neuron development. Ann N Y Acad Sci 991, 48-60.

Wallen-Mackenzie, A., Mata de Urquiza, A., Petersson, S., Rodriguez, F. J., Friling, S., Wagner, J., Ordentlich, P., Lengqvist, J., Heyman, R. A., Arenas, E., and Perlmann, T. (2003). Nurr1-RXR heterodimers mediate RXR ligand-induced signaling in neuronal cells. Genes Dev 17, 3036-3047.

Walrand S, Valeix S, Rodriguez C, Ligot P, Chassagne J, et al. (2003) Flow cytometry study of polymorphonuclear neutrophil oxidative burst: a comparison of three fluorescent probes. Clin Chim Acta 331(1-2): 103-110.

Wang, Z., Benoit, G., Liu, J., Prasad, S., Aarnisalo, P., Liu, X., Xu, H., Walker, N. P., and Perlmann, T. (2003). Structure and function of Nurr1 identifies a class of ligand-independent nuclear receptors. Nature 423, 555-560.

Wichterle, H., Lieberam, I., Porter, J. A., and Jessell, T. M. (2002). Directed differentiation of embryonic stem cells into motor neurons. Cell 110, 385-397.

Wilson, S. I., Rydstrom, A., Trimborn, T., Willert, K., Nusse, R., Jessell, T. M., and Edlund, T. (2001). The status of Wnt signalling regulates neural and epidermal fates in the chick embryo. Nature 411, 325-330.

Witta, J., Baffi, J. S., Palkovits, M., Mezey, E., Castillo, S. O., and Nikodem, V. M. (2000). Nigrostriatal innervation is preserved in Nurr1-null mice, although dopaminergic neuron precursors are arrested from terminal differentiation. Brain Res Mol Brain Res 84, 67-78.

Yan, Y. et al. Directed differentiation of dopaminergic neuronal subtypes from human embryonic stem cells. Stem Cells 23, 781-90 (2005).

Yang L, Matthews R T, Schulz J B, Klockgether T, Liao A W, et al. (1998) 1-Methyl-4-phenyl-1,2,3,6-tetrahydropyride neurotoxicity is attenuated in mice overexpressing Bcl-2. J Neurosci 18(20): 8145-8152.

Ye, W., Bouchard, M., Stone, D., Liu, X., Vella, F., Lee, J., Nakamura, H., Ang, S. L., Busslinger, M., and Rosenthal, A. (2001). Distinct regulators control the expression of the mid-hindbrain organizer signal FGF-8. Nat Neurosci 4, 1175-1181.

Ye, W., Shimamura, K., Rubenstein, J. L., Hynes, M. A., and Rosenthal, A. (1998). FGF and Shh signals control dopaminergic and serotonergic cell fate in the anterior neural plate. Cell 93, 755-766.

Yokota T, Sugawara K, Ito K, Takahashi R, Ariga H, et al. (2003) Down regulation of DJ-1 enhances cell death by oxidative stress, ER stress, and proteasome inhibition. Biochem Biophys Res Commun 312(4): 1342-1348.

Yu, Y. et al. The nuclear hormone receptor Ftz-F1 is a cofactor for the Drosophila homeodomain protein Ftz. Nature 385, 552-5 (1997).

Yue, Y., Widmer, D. A., Halladay, A. K., Cerretti, D. P., Wagner, G. C., Dreyer, J. L., and Zhou, R. (1999). Specification of distinct dopaminergic neural pathways: roles of the Eph family receptor EphB1 and ligand ephrin-B2. J Neurosci 19, 2090-2101.

Zennou, V., Serguera, C., Sarkis, C., Colin, P., Perret, E., Mallet, J., and Charneau, P. (2001). The HIV-1 DNA flap stimulates HIV vector-mediated cell transduction in the brain. Nat Biotechnol 19, 446-450.

Zetterstrom, R. H., Solomin, L., Jansson, L., Hoffer, B. J., Olson, L., and Perlmann, T. (1997). Dopamine neuron agenesis in Nurr1-deficient mice. Science 276, 248-250.

Zhuang, X., Oosting, R. S., Jones, S. R., Gainetdinov, R. R., Miller, G. W., Caron, M. G., and Hen, R. (2001). Hyperactivity and impaired response habituation in hyperdopaminergic mice. Proc Natl Acad Sci USA 98, 1982-1987.

Zhuang, X., Masson, J., Gingrich, J. A., Rayport, S. & Hen, R. Targeted gene expression in dopamine and serotonin neurons of the mouse brain. J Neurosci Methods 143, 27-32 (2005).

What is claimed is:

1. A method for promoting differentiation of a stem cell to a dopamine-producing neuron, comprising co-expressing Nurr1 and PitX3 in the stem cell, thereby increasing the responsiveness of the stem cell to one or more differentiation-inducing stimuli.

2. A method for promoting maturation or survival of a dopamine-producing neuron differentiated from a stem cell, comprising co-expressing Nurr1 and PitX3 in the stem cell, thereby increasing the responsiveness of the stem cell to one or more differentiation-inducing stimuli.

3. The method of claim 1 or 2, wherein the stem cell is an adult stem cell or somatic stem cell.

4. The method of claim 1 or 2, wherein the stem cell is an embryonic stem cell.

5. The method of claim 1 or 2, wherein the stem cell is a mammalian stem cell.

6. The method of claim 1 or 2, wherein the stem cell is a murine stem cell.

7. The method of claim 1 or 2, wherein the stem cell is a human stem cell.

8. A method for promoting differentiation of an embryonic stem cell to a dopamine-producing neuron, comprising co-expressing Nurr1 and PitX3 in the embryonic stem cell, thereby increasing the responsiveness of the embryonic stem cell to one or more differentiation-inducing stimuli.

9. A method for promoting maturation or survival of a dopamine-producing neuron differentiated from an embryonic stem cell, comprising co-expressing Nurr1 and PitX3 in the embryonic stem cell, thereby increasing the responsiveness of the embryonic stem cell to one or more differentiation-inducing stimuli.

10. The method of claim 8 or 9, wherein the embryonic stem cell is a mammalian embryonic stem cell.

11. The method of claim 8 or 9, wherein the embryonic stem cell is a murine embryonic stem cell.

12. The method of claim 8 or 9, wherein the embryonic stem cell is a human embryonic stem cell.

13. The method of claim 8 or 9, wherein the differentiation-inducing stimulus comprises brain-derived neurotrophic factor (BDNF), glial cell-line-derived neurotrophic factor (GDNF), sonic hedgehog (SHH), fibroblast growth factor-8 (FGF-8), jagged-1, neuregulin-1β, or any combination thereof.

14. A method for producing a dopamine-producing neuron, comprising:
1. co-expressing Nurr1 and PitX3 in an embryonic stem cell; and
2. contacting the embryonic stem cell with one or more differentiation-inducing stimuli, wherein the embryonic stem cell is capable of differentiating into the dopamine-producing neuron.

15. A method for producing a dopamine-producing neuron, comprising:
3. co-expressing Nurr1 and PitX3 in an adult stem cell or a somatic stem cell; and
4. contacting the stem cell with one or more differentiation-inducing stimuli,
wherein the stem cell is capable of differentiating into the dopamine-producing neuron.

16. The method of claim 14 or 15, wherein the co-expressing comprises transfecting the stem cell with (i) a vector comprising a nucleotide sequence encoding Nurr1; and (ii) a vector comprising a nucleotide sequence encoding PitX3, wherein Nurr1 and PitX3 are co-expressed in the stem cell under appropriate gene expression conditions.

17. The method of claim 16, wherein the vector comprises a viral vector.

18. The method of claim 16, wherein the vector comprises a lentiviral vector.

19. The method of claim 14 or 15, wherein the co-expressing comprises transfecting the stem cell with a vector comprising (i) a nucleotide sequence encoding Nurr1; and (ii) a nucleotide sequence encoding PitX3, wherein Nurr1 and PitX3 are co-expressed in the stem cell under appropriate gene expression conditions.

20. The method of claim 19, wherein the vector comprises a viral vector.

21. The method of claim 19, wherein the vector comprises a lentiviral vector.

22. The method of claim 14 or 15, wherein the differentiation-inducing stimulus comprises brain-derived neurotrophic factor (BDNF), glial cell-line-derived neurotrophic factor (GDNF), sonic hedgehog (SHH), fibroblast growth factor-8 (FGF-8), jagged-1, neuregulin-1β, or any combination thereof.

23. The method of claim 14, wherein the embryonic stem cell comprises an embryoid body.

24. The method of claim 14, wherein the embryoid body is in or around stage 3.

25. An isolated stem cell co-expressing Nurr1 and PitX3 from one or more nucleic acid expression vectors contained therein, wherein the stem cell is capable of differentiating into a dopamine-producing neuron.

26. The method of claim 25, wherein the stem cell is an embryonic stem cell.

27. The method of claim 25, wherein the stem cell is an adult stem cell or a somatic stem cell.

28. The method of claim 25, wherein the stem cell is a human stem cell.

29. An isolated dopamine-producing neuron co-expressing Nurr1 and PitX3 from one or more nucleic acid expression vectors contained therein, wherein the dopamine producing neuron is differentiated from an isolated stem cell.

30. A method for replacing dopamine neurons in a subject with a neuron-associated disorder, the method comprising administering to the subject embryonic stem cells co-expressing Nurr1 and PitX3 from one or more vectors contained therein.

31. A method for replacing dopamine neurons in a subject with a neuron-associated disorder, the method comprising administering to the subject adult stem cells co-expressing Nurr1 and PitX3 from one or more vectors contained therein.

32. A method for replacing dopamine neurons in a subject with a neuron-associated disorder, the method comprising administering to the subject dopamine neurons differentiated from embryonic stem cells co-expressing Nurr1 and PitX3 from one or more vectors contained therein.

33. A method for replacing dopamine neurons in a subject with a neuron-associated disorder, the method comprising administering to the subject dopamine neurons differentiated from adult stem cells or somatic stem cells co-expressing Nurr1 and PitX3 from one or more vectors contained therein.

34. A method for treating or preventing a neuron-associated disorder in a subject in need thereof, the method comprising upregulating the expression of Nurr1 and PitX3 in the subject.

35. The method of claim 29, 30, 31, 32, 33, or 34, wherein the neuron-associated disorder comprises a brain tumor, a developmental disorder, a neurodegenerative disease, or a seizure disorder.

36. The method of claim 35, wherein the neurodegenerative disease comprises Alzheimer's disease, amyotrophic lateral sclerosis (Lou Gehrig's disease), Binswanger's disease, Huntington's chorea, multiple sclerosis, myasthenia gravis, Parkinson's disease, or Pick's disease.

37. The method of claim 29, 30, 31, 32, 33, or 34, wherein the neuron-associated disorder comprises Parkinson's disease.

38. A method for identifying whether a test compound is capable of enhancing the differentiation of a stem cell expressing Nurr1 and PitX3 to a dopamine-producing neuron, the method comprising:
(a) contacting a stem cell expressing Nurr1 and PitX3 with a test compound; and
(b) determining whether differentiation of the stem cell in (a) is enhanced compared to the differentiation of a stem cell expressing Nurr1 and PitX3 in the absence of the test compound, so as to identify whether the test compound is capable of enhancing the differentiation of the stem cell to a dopamine-producing neuron.

39. A method for identifying whether a test compound is capable of upregulating Nurr1 and PitX3 activity in a stem cell, the method comprising:
(a) contacting a stem cell with a test compound; and
(b) determining whether activity of Nurr1 and PitX3 are upregulated in the stem cell of (a) compared the activity of Nurr1 and PitX3 in a stem cell in the absence of the test compound, so as to identify whether the test compound is capable of upregulating the activity of Nurr1 and PitX3 in the stem cell.

40. A transgenic non-human mammal whose somatic and germ cells comprise:
(a) a DNA segment comprising a nucleotide sequence encoding a Nurr1; and
(b) a DNA segment comprising a nucleotide sequence encoding a PitX3, wherein the DNA segments are capable of producing Nurr1 and PitX3 under appropriate expression conditions in the transgenic non-human mammal.

41. A transgenic non-human mammal whose somatic and germ cells are deficient in one or more genes encoding Nurr1 and PitX3, wherein the transgenic non-human mammal has diminished expression of Nurr1 and PitX3 compared to a wildtype counterpart of the transgenic non-human mammal.
